SEQUENCE LISTING (13 sequences)

SEQ ID NO: 1 - DNA, 1500 bp, Artificial Sequence
  Description of Artificial Sequence Synthetic scTNF alpha
gttagatcttcttcaagaactccatctgataaaccagttgctcatgttgttgctaatccacaagctgaaggtcaattgcaatggttgaatagaagagctaatgctttgttggctaatggtgttgaattgagagataatcaattggttgttccatctgaaggtttgtatttgatttattctcaagttttgtttaaaggtcaaggttgtccatctactcatgttttgttgactcatactatttcaagaattgctgtttcttatcaaactaaagttaatttgttgtctgctattaaatctccatgtcaaagagaaactccagaaggtgctgaagctaaaccatggtatgaaccaatttatttgggtggtgtttttcaattggaaaaaggtgatagattgtctgctgaaattaatagaccagattatttggattttgctgaatctggtcaagtttattttggtattattgccctaggttctacttcaggttctggaaaatcatctgaaggtaaaggcgtacgttcatcctcacgtacaccctcagacaaacccgtagcccatgtcgtagccaatccccaagccgaaggccaactacaatggctaaatcgtagggccaatgccttactagcaaacggagtcgaactaagggacaatcagttagtcgtaccctcagagggactatatctaatatactcacaggtactatttaagggacagggatgcccttccacacacgtactattaacccataccatatccaggatcgcagtctcataccaaaccaaagtcaatctattatccgcaatcaaatccccttgccaacgtgaaacccctgagggagccgaagccaaaccctggtatgagcctatctatctaggaggcgtattccaactagaaaagggagacaggctatcagccgaaatcaatcgtcctgactatttagacttcgcagagtcaggccaagtctatttcggaatcatagccttaggatcaacatctggatcaggtaagtcttcagaaggaaaaggagtcaggtcctcatccaggaccccttccgataaaccggtcgcacacgtagtcgcaaaccctcaggcagagggacagttacagtggttaaacaggcgtgcaaacgcactattagccaatggcgtagagttacgtgataaccaactagtagtcccttccgaaggcttatacttaatctattcccaagtcttattcaaaggccaaggctgtccctcaacccatgtcttactaacacacacaatctcacgtatagccgtatcctatcagacaaaggtaaacttactatcagccataaagtcaccctgtcagagggagacacccgaaggcgcagaggcaaagccttggtacgaacccatatacttaggcggagtctttcagttagagaaaggcgatcgtttatccgcagagataaacaggcccgattacttagactttgccgaatccggacaggtatactttggcataatcgcactataa

SEQ ID NO: 2 - protein, 499 aa, Artificial Sequence
  Description of Artificial Sequence Synthetic scTNF alpha
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGSTSGSGKSSEGKGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGSTSGSGKSSEGKGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 3 - protein, 244 aa, Homo sapiens
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 4 - protein, 5 aa, Artificial Sequence
  Description of Artificial Sequence Linker
GGGGS

SEQ ID NO: 5 - protein, 6 aa, Artificial Sequence
  Description of Artificial Sequence Synthetic tag
HHHHHH

SEQ ID NO: 6 - protein, 8 aa, Artificial Sequence
  Description of Artificial Sequence Synthetic tag
MKHHHHHH

SEQ ID NO: 7 - protein, 10 aa, Artificial Sequence
  Description of Artificial Sequence Synthetic tag
MKHHAHHQHH

SEQ ID NO: 8 - protein, 14 aa, Artificial Sequence
  Description of Artificial Sequence Synthetic tag
MKHQHQHQHQHQHQ

SEQ ID NO: 9 - protein, 144 aa, Artificial Sequence
  Description of Artificial Sequence Synthetic LT-alpha peptide
KPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 10 - protein, 152 aa, Artificial Sequence
  Description of Artificial Sequence Synthetic TNF-alpha peptide
RTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLLFAESGQVYFGIIAL

SEQ ID NO: 11 - protein, 172 aa, Artificial Sequence
  Description of Artificial Sequence Synthetic LT-beta peptide
KLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 12 - DNA, 1770 bp, Saccharomyces cerevisiae
  CDS (10)..(1761)
ggatccacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaccagcacaaataacgggttattgtttataaatactactattgccagcattgctgctaaagaagaaggggtatctctcgagaaaagagttagatcttcttcaagaactccatctgataaaccagttgctcatgttgttgctaatccacaagctgaaggtcaattgcaatggttgaatagaagagctaatgctttgttggctaatggtgttgaattgagagataatcaattggttgttccatctgaaggtttgtatttgatttattctcaagttttgtttaaaggtcaaggttgtccatctactcatgttttgttgactcatactatttcaagaattgctgtttcttatcaaactaaagttaatttgttgtctgctattaaatctccatgtcaaagagaaactccagaaggtgctgaagctaaaccatggtatgaaccaatttatttgggtggtgtttttcaattggaaaaaggtgatagattgtctgctgaaattaatagaccagattatttggattttgctgaatctggtcaagtttattttggtattattgccctaggttctacttcaggttctggaaaatcatctgaaggtaaaggcgtacgttcatcctcacgtacaccctcagacaaacccgtcgcccatgtcgtagccaatccccaagccgaaggccaactacaatggctaaatcgtagggccaatgccttactagcaaacggagtcgaactaagggacaatcagttagtcgtaccctcagagggactgtatctaatatactcacaggtactatttaagggacagggatgcccttccacacacgtactattaacccataccatatccaggatcgcagtctcataccaaaccaaagtcaatctattatccgcaatcaaatccccttgccaacgtgaaacccctgagggagccgaagccaaaccctggtatgagcctatctatctaggaggcgtattccaactagaaaagggagacaggctatcagccgaaatcaatcgtcctgactatttagacttcgcagagtcaggccaagtctatttcggaatcatagccttaggatcaacatctggatcaggtaagtcttcagaaggaaaaggagtcaggtcctcatccaggaccccttccgataaacctgtcgcacacgtagtcgcaaaccctcaggcagagggacagttacagtggttaaacaggcgtgcaaacgcactattagccaatggcgtagagttacgtgataaccaactagtagtcccttccgaaggcttatacttaatctattcccaagtcttattcaaaggccaaggctgtccctcaacccatgtcttactaacacacacaatctcacgtatagccgtatcctatcagacaaaggtaaacttactatcagccataaagtcaccctgtcagagggagacacccgaaggcgcagaggcaaagccttggtacgaacccatatacttagggggagtctttcagttagagaaaggcgatcgtttatccgcagagataaacaggcccgattacttagactttgccgaatccggacaggtatactttggcataatcgcactataatctaga

SEQ ID NO: 13 - protein, 584 aa, Saccharomyces cerevisiae
MRFPSIFTAVLFAASSALAAPVNTTTEDETAQIPAEAVIGYSDLEGDFDVAVLPFSTSTNNGLLFINTTIASIAAKEEGVSLEKRVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGSTSGSGKSSEGKGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGSTSGSGKSSEGKGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

(19) United States
(12) Patent Application Publication
(10) Pub. No.: US 2004/0014948 A1
(43) Pub. Date: Jan. 22, 2004

(54) Single-chain antagonist polypeptides

(75) Inventors: Torben Halkier, Solroed Strand (DK); Hans Thalsgard Schambye, Holte (DK); Jens Sigurd Okkels, Vedbaek (DK); Kim Vilbour Andersen, Broenshoej (DK); Torben Lauesgaard Nissen, San Francisco, CA (US); Bobby Soni, Copenhagen K. (DK); Claus Bekker Jeppesen, Nivaa (DK); Bart van den Hazel, Copenhagen (DK)

Correspondence Address: MAXYGEN, INC., INTELLECTUAL PROPERTY DEPARTMENT, 515 GALVESTON DRIVE, RED WOOD CITY, CA, 94063, US

(73) Assignee: Maxygen ApS

(21) Appl. No.: 10/444,691

(22) Filed: May 23, 2003

Related U.S. Application Data
(63) Continuation of application No. 09/684,720, filed on Oct. 6, 2000, now abandoned.
(60) Provisional application No. 60/160,820, filed on Oct. 21, 1999.
(60) Provisional application No. 60/174,655, filed on Jan. 6, 2000.
(60) Provisional application No. 60/225,723, filed on Aug. 16, 2000.

(30) Foreign Application Priority Data
Jul. 20, 2000 (DK) PA 2000 01119
Dec. 23, 1999 (DK) PA 1999 01855
Oct. 7, 1999 (DK) PA 1999 01438

(51) Int. Cl. C07K 16/28

(52) U.S. Cl. 530/388.22; 530/324

(57) ABSTRACT

The invention relates to a single-chain oilgomeric polypeptide antagonist which binds to an extracellular ligand-binding domain of a cellular receptor of a type requiring binding of an oligomeric ligand to two or more receptor subunits to be activated, the polypeptide comprising at least two, typically structurally homologous, receptor-binding sites of which at least one is capable of binding to a ligand-binding domain of the cellular receptor and at least one is incapable of effectively binding to a ligand-binding domain of the cellular receptor, whereby the single-chain oligomeric polypeptide is capable of binding to the receptor, but incapable of activating the receptor; as well as to nucleotide sequences encoding such single-chain oligomeric polypeptides, expression vectors comprising such a nucleotide sequence, recombinant host cells comprising such a nucleotide sequence or expression vector, methods for producing the nucleotide sequences and polypeptides, pharmaceutical compositions comprising the single-chain oligomeric polypeptides, and use of the single-chain oligomeric polypeptides for the production of medicaments and in therapy. A preferred single-chain antagonist according to the invention is a TNF-α antagonist.

### CROSS-REFERENCES TO RELATED APPLICATIONS

Pursuant to 35 USC 119 and 120, and any other applicable statute or rule, this application claims the benefit of and priority from each of the following Application Numbers/filing dates: Denmark Patent Application No. PA 1999 01438, filed Oct. 7, 1999; U.S. Serial No. 60/160,820, filed Oct. 21, 1999; Denmark Patent Application No. PA 1999 01855, filed Dec. 23, 1999; U.S. Serial No. 60/174,655, filed Jan. 6, 2000; Denmark Patent Application No. PA 2000 01119, filed Jul. 20, 2000; and U.S. Serial No. 60/225,723, filed Aug. 16, 2000, the disclosures of which are incorporated by reference.

### COPYRIGHT NOTIFICATION PURSUANT TO 37 C.F.R. § 1.71(e)

A portion of the disclosure of this patent document contains material which is subject to copyright protection. The copyright owner has not objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates to single chain oligomeric polypeptides, in particular polypeptides with receptor antagonist activity, as well as to nucleotide sequences encoding such single-chain oligomeric polypeptides, expression vectors comprising such a nucleotide sequence, recombinant host cells comprising such a nucleotide sequence or expression vector, methods for producing the nucleotide sequences and polypeptides, pharmaceutical compositions comprising the single-chain oligomeric polypeptides, and use of the single-chain oligomeric polypeptides for the production of medicaments and in, therapy.

### BACKGROUND OF THE INVENTION

It has long been recognised that several polypeptide ligands, such as cytokines and growth factors, act by binding to extracellular portions of cell surface receptors, and that such receptors are often activated by ligand-induced oligomerisation or conformational changes. This mechanism of action has been comprehensively reviewed by Heldin (Heldin, C. -H. (1995) Dimerization of cell surface receptors in signal transduction. Cell 80, 213-223.). In this reference, it is indicated that antagonists of the receptors may be prepared either by expression of mutated receptors in cells where the mutants dimerise with the wild-type receptors to form inactive heteromeric complexes, or by mutating and thus inactivating the receptor-binding region of the ligand.

Many experiments conducted on the inactivation of receptors of this type have involved the expression of the ligand of the receptor in question in monomeric form or, in case of heterodimers in particular, the genes coding for each monomer have been expressed and purified separately, followed by mixing the two monomers under denaturing conditions to make them combine. This procedure then necessitated refolding procedures to take place as well as purification to remove remaining monomers and possible homodimers (e.g. as described in G. Siemeister et al., Proc. Natl. Acad. Sci. USA 95, 1998, pp. 4625-4629; and G. Fuh et al., J. Biol. Chem. 273, 1998, pp. 11197-11204).

To simplify production procedures, it has in some instances been proposed to produce oligomeric polypeptide ligands as single-chain polypeptides, i.e. expressed in a cell from a single nucleotide sequence. Thus, WO 96/05224 describes single-chain forms of the glycoprotein hormones LH, FSH, TSH and CG and variants thereof in which the amino acid sequence of the native proteins has been altered by deletion, insertion or substitution. These hormones, however, act on a different type of receptors (G-protein coupled receptors with seven transmembrane domains) which are not activated by oligomerisation, but rather by G-protein mediated signal transduction, and it is specifically indicated that alterations do not occur in the receptor-binding portions as this is believed to destroy any activity, including antagonist activity, of such variants.

Interleukin-8 (IL-8) is a normally homo-dimeric protein which has been expressed as a single polypeptide chain (Leong et al. (1997), Protein Sci. 6, 609-617). The IL-8 receptors, however, are also G-protein coupled receptors and thus not activated through oligomerisation.

Interleukin-12 (IL-12) is a normally hetero-dimeric protein which has been expressed as a single polypeptide chain (Lieschke et al. (1997), Nat. Biotechnol. 15, 35-40). Other examples of normally dimeric protein which have been expressed as a single polypeptide chain include interleukin-5 (IL-5) (Li et al. (1996a), J. Biol. Chem. 271, 1817-1820; Li et al. (1996b), J. Biol. Chem. 271, 31729-31734) and interferon-γ (IFN-γ) (Lunn et al. (1992), J. Biol. Chem. 267, 17920-17924; Randal and Kossiakoff (1998), Protein Sci. 7, 1057-1060).

U.S. Pat. 6,022,711 describes specific variants of human growth hormone (hGH), which is a monomeric hormone binding two receptor molecules sequentially through distinct binding sites. The variants have a substantially increased receptor-binding activity at one of the two receptor-binding sites. Modification of one of the two receptor-binding sites of hGH is also disclosed by Fuh et al. in Science 256: 1677-1680 (1992).

U.S. Pat. No. 6,057,428 and WO 97/08313 disclose the production of VEGF variants having mutations in the Kinase domain region (KDR) and/or the FMS-like Tyrosine-Kinase region (FLT-1) to result in modified binding characteristics compared to the native VEGF. It is disclosed that VEGF antagonists can in theory be produced by making a VEGF homodimer into a single chain molecule, but no single chain VEGF antagonists are actually exemplified.

WO 98/27230 discloses several methods for evolving new proteins using recursive sequence recombination (RSR). It is disclosed that in some embodiments, the substrate for evolution by RSR is a single chain version of a multisubunit factor. Various polypeptide candidates for evolution are listed in Table 1 therein.

### SUMMARY OF THE INVENTION

It has now been found that it is possible to modify polypeptide ligands which bind to cellular receptors that depend on oligomerisation and/or conformational changes to be activated, within a receptor-binding site, in a way that does not destroy the ability of the ligand to bind to the receptor, but destroys the ability of the ligand to activate the receptor.

Accordingly, the present invention relates, in one aspect, to a single-chain oligomeric polypeptide which binds to an extracellular ligand-binding domain of a cellular receptor of a type requiring binding of an oligomeric ligand to two or more receptor subunits to be activated, the polypeptide comprising at least three receptor-binding sites of which at least one is capable of binding to a ligand-binding domain of the cellular receptor and at least one is incapable of effectively binding to a ligand-binding domain of the cellular receptor, whereby the single-chain oligomeric polypeptide is capable of binding to the receptor, but incapable of activating the receptor.

The invention further relates to a single-chain oligomeric polypeptide which binds to an extracellular ligand-binding domain of a cellular receptor of a type requiring binding of an oligomeric ligand to two or more receptor subunits to be activated, the polypeptide comprising at least two structurally homologous receptor-binding sites of which at least one is capable of binding to a ligand-binding domain of the cellular receptor and at least one is incapable of effectively binding to a ligand-binding domain of the cellular receptor, whereby the single-chain oligomeric polypeptide is capable of binding to the receptor, but incapable of activating the receptor.

In another aspect, the invention relates to a nucleotide sequence encoding such polypeptide, an expression vector and a host cell comprising such nucleotide sequence and a method for preparing such a polypeptide by recombinant DNA techniques.

In a further aspect, the invention relates to a pharmaceutical composition comprising such a polypeptide together with a pharmaceutically acceptable excipient or vehicle.

In a still further aspect, the invention relates to the use in therapy of a single-chain oligomeric polypeptide of the invention, in particular for use as an antagonist of an oligomeric cellular receptor in the prevention or treatment of a disease or condition involving an undesired, e.g. increased signal transduction from or an undesired, e.g. increased activation of the receptor.

In a still further aspect, the invention relates to a method for identifying suitable single-chain oligomeric polypeptides of the present invention, the method comprising contacting one or more test polypeptides with (a cell expressing) an appropriate cellular receptor for the oligomeric polypeptide in question and identifying polypeptides which bind to and inhibit activation of said receptor.

The present invention differs from previously reported examples of the use of single-chain versions of otherwise homo- or hetero-oligomeric proteins resides in part in the structure of the receptor-binding regions of the proteins. In the previously reported examples (IL-12, IL-5, and IFN-γ) ligand-induced oligomerisation is part of the signalling pathway. However, these are dimeric proteins in which receptor-binding regions required for receptor oligomerisation and activation are not structurally homologous.

### BRIEF DESCRIPTION OF THE DRAWINGS

FIG. 1 is a dose-response curve showing the agonistic effect of monomeric wild-type TNF-α, a single-chain trimeric protein comprising three copies of wild-type TNF-α, and a single-chain trimeric antagonist protein according to the invention comprising two modified copies of TNF-α and one copy of wild-type TNF-α.

FIG. 2 shows the antagonistic effect of the modified single-chain trimeric protein of the invention.

FIG. 3 is a competitive dose-response curve illustrating the antagonistic effect of the modified single-chain trimeric protein of the invention.

FIG. 4 shows the complete DNA and amino acid sequence of a single-chain trimeric protein construct comprising three copies of wild-type TNF-α.

### DETAILED DESCRIPTION OF THE INVENTION

In the present context, the term “polypeptide” is understood to indicate a mature protein or a precursor form thereof as well as a functional fragment thereof which essentially has retained the activity of the mature protein, i.e. exhibits at least the same qualitative activity and preferably also at least the same quantitative activity as the mature protein. A functional fragment may for instance be an N- and/or C-terminal truncated form of a full-length polypeptide, or an isoform in particular a native isoform, of a full-length polypeptide.

The polypeptides of the invention are derived from or otherwise made so as to mimic the structure and function of parent polypeptides which in their native form are oligomers, i.e. they are composed of two, three or more monomeric subunits which are connected by for instance disulfide bonds or non-covalent bonds.

In the present context the term “derived” is intended to indicate that the monomeric polypeptide subunit is prepared to mimic structural and/or functional properties of the corresponding native polypeptide in question. The “derived” polypeptide may have the same amino acid sequence as said native polypeptide, except for an optional limited number of amino acid changes, e.g. those made in accordance with the present invention. Thus, typically, the amino acid sequence of the “derived” polypeptide is at least 60% identical to that of said native polypeptide, normally at least 70% identical, such as at least 80% or even at least 90% identical. Also, the “derived” polypeptide may share a number of functional and/or structural properties with said native polypeptide, in particular one or more of the properties discussed herein, especially in terms of receptor-binding properties and/or oligomer association properties and/or conformation of a receptor-binding site and/or an association domain thereof. Typically, the monomeric polypeptide is encoded by the same nucleotide sequence as the corresponding native monomer or from a nucleotide sequence which is able to express a polypeptide with the same amino acid sequence as the corresponding native monomer, or from any such nucleotide sequence which has been modified so that the monomeric polypeptide is expressed with one or more desirable mutations, e.g. as described herein. Preferably, the monomeric polypeptides used in the present invention are of mammalian origin, in particular of human origin.

The term “parent polypeptide” is used about the usually native oligomeric polypeptide, which in accordance with the present invention is provided in a modified single-chain form. The monomers may be identical (in which case the polypeptide is termed a “homomer”) or different (in which case the polypeptide is termed a “heteromer”). According to the invention, the polypeptides are provided in single-chain form, which means that the monomers are linked by peptide bonds, optionally through a linker peptide, rather than being linked by non-covalent bonds or disulfide bonds. Accordingly, the single-chain polypeptides of the invention are expressed as one polypeptide from a single nucleotide sequence rather than being expressed as single monomer molecules which are assembled to an oligomeric polypeptide only after expression. The parent monomer may be a wild-type monomeric polypeptide or a variant thereof, for instance a mutein form of the wild-type monomeric polypeptide which has been prepared by substitution or deletion of one or more amino acid residues thereof and/or insertion of one or more additional amino acid residues therein.

In the present context, the term “oligomeric polypeptide” is merely intended to indicate that the polypeptide is of a type which is oligomeric in its native state (i.e. forms a dimer, trimer, etc.) since, strictly speaking, the single-chain form of the polypeptide cannot be said to be oligomeric. The terms “dimer”, “dimeric”, “trimer”, “trimeric”, etc. are used in the same manner. The term “signalling polypeptide” is used to denote a polypeptide that interacts with a cellular receptor so as to activate the receptor and thereby provide a signal initiating a signal transduction cascade in the cell carrying the receptor. Such a polypeptide is often also termed a ligand.

In the present context, an “antagonist” is a molecule which is capable of binding to a desired receptor but incapable of mediating correct conformational changes of the receptor molecules necessary to result in an activated complex, whereby ligand-mediated receptor activation is substantially inhibited. In order to efficiently inhibit receptor activation, the single chain polypeptide must be capable of binding to a ligand-binding domain of a receptor with a sufficiently high affinity to compete with the endogenous ligand. In order to be effective, the single-chain antagonist of the invention normally has at least one receptor-binding site which is inactive and at least one which is active.

One very important advantage of the present invention is that by providing oligomeric polypeptides comprising at least two or three receptor-binding sites in single-chain form expressed from one continuous nucleotide sequence it is possible to selectively modify at least one receptor-binding site, and leave the other binding site(s) intact through assymetrical mutagenesis. This is in contrast to the non-single-chain situation, wherein monomers are expressed individually from the same or different genes and subsequently assembled in the cell. For instance, in the case of a homotrimer, modification of the gene encoding the monomer through mutagenesis will always be symmetrical. Accordingly, the production of an oligomeric non-single-chain polypeptide with at least one intact and at least one modified receptor-binding site requires that the monomers must be modified and produced separately before in vitro recombination. Following recombination, purification of the desired oligomeric non-single-chain polypeptide has to be carried out in order to separate it from non-desired oligomer. Thus, it is very laborious, if possible at all, to produce a preparation of oligomeric non-single-chain polypeptides each of which has at least one intact and at least one modified receptor-binding site, since this requires that monomers having the appropriate receptor-binding sites be assembled correctly.

In addition, the single-chain form of the polypeptides more readily lends itself to production by recombinant DNA techniques in that the polypeptides may be expressed from a single gene rather than being assembled in the cell from two or more individual monomers (homomers) or, in the case of heteromers, expressed from two or more individual genes and assembled in the cell as is often the case with oligomeric polypeptides in nature (cf., for instance, Siemeister et al., supra). Single-chain polypeptides may have the added advantage of greater stability upon administration, for instance against degradation by proteolytic enzymes present in the body, e.g. in plasma, so that they may exhibit a longer half-life in vivo.

In the present context the term “active receptor-binding site” is intended to indicate a receptor-binding site which is capable of binding to a ligand-binding domain of the cellular receptor in question, i.e. a cellular receptor of a type requiring oligomerisation or other conformational change via ligand binding to be activated. The active receptor-binding site has a sufficient affinity towards the ligand-binding domain of the receptor to effect binding between the receptor-binding site and the receptor and thereby to block the receptor from binding to, e.g., a native oligomeric polypeptide (an endogenous ligand), thus preventing subsequent activation by the native polypeptide. This affinity is preferably the same as or higher than the affinity of the native polypeptide.

The term “inactive receptor-binding site” is intended to indicate a receptor-binding site which renders the single-chain oligomeric polypeptide incapable of activating the receptor. Usually, an inactive receptor-binding site is incapable of effectively binding to a ligand-binding domain of the cellular receptor in question. The term “incapable of effectively binding” is intended to indicate that the interaction between the binding site of the ligand and the binding site of the receptor is unable to mediate correct receptor oligomerisation or other conformational change required for activation and thus to trigger a signal transduction cascade within the cell. This can be due to an inability of the receptor-binding site to recognize and/or bind the ligand-binding site on the receptor or due to an imperfect binding, which does not permit correct interaction between the intracellular parts of the oligomerised receptor subunits. Accordingly, the inactive receptor-binding site may exhibit affinity towards the receptor, which however, under normal concentrations of receptor-binding site, is insignificant as a means of inducing receptor oligomerisation. Preferably, the inactive receptor-binding site has no affinity towards the receptor and is thus incapable of binding thereto. Although the term “receptor-binding site” is used in connection with the inactive receptor-binding site, strictly speaking, the inactive “receptor-binding site” may not be capable of binding to the receptor. Thus, in the case of an inactive receptor-binding site, the term “receptor-binding site” is merely used to reflect that said site is derived from an active receptor-binding site in accordance with the present invention, typically by modifying one or more amino acid residues thereof or by addition of a non-polypeptide moiety, said modification leading to the inactivation of the binding site.

The term “ligand-binding domain” refers to the part or parts of a cellular receptor which is/are involved in specific recognition of and interaction with a receptor-binding site of an endogenous ligand. Analogously, the “receptor-binding site” is understood as a number of amino acid residues involved in polypeptide binding to the ligand-binding domain of the receptor. Normally, the receptor-binding site comprises 1-50 amino acid residues, such as 5-30 or 10-25 amino acid residues. The amino acid residues in question may be located in sequence, but are more often placed in spatial proximity to each other as a result of the folding of the polypeptide. Although a receptor-binding site may contain amino acid residues originating from only one monomer, the receptor-binding site of interest for the present invention preferably includes one or more amino acid residues originating from a first monomer and one or more amino acid residues originating from a second monomer of the oligomeric polypeptide. More specifically, the receptor-binding sites may be located at interfaces between the monomeric constituents of the oligomeric polypeptide. For TNF-α, for example, the receptor-binding site includes amino acid residues located in positions 53, 71, 72, 73, 74, 75, 77, 82, 84, 85, 86, 87, 88, 89, 91, 97, 125, 127, 137 and 138 in a first monomer and amino acid residues located in positions 6, 17, 20, 21, 23, 29, 30, 31, 32, 33, 34, 35, 63, 65, 66, 67, 110, 111, 112, 113, 114, 115, 139, 140, 142, 143, 144, 145, 146, 147, and 149 in the subsequent monomer. In the Materials and Methods section below, a method is described for determining residues of a receptor-binding site.

The term “structurally homologous” used in this context is understood to indicate that the receptor-binding sites have a very similar and preferably the same overall three-dimensional structure and/or that amino acid sequences of each receptor-binding site are homologous, i.e. sufficiently identical to be aligned. This similarity is usually obtained by a high degree of identity of the amino acid residues within the two receptor-binding sites in question. Normally, at least 50%, such as at least 70%, at least 85%, at least 90% or at least 95% of the amino acid residues within one receptor-binding site responsible for binding to the receptor should be identical to the amino acid residues at the same positions within the other receptor-binding site(s) in order to provide a satisfactory overall structural homology. Expressed differently, it is preferred that if a three-dimensional image of one receptor-binding site is superimposed on another receptor-binding site, there should be a virtually complete overlap between the three-dimensional structures. Consequently, in the parent oligomeric signalling polypeptide both sites have a structurally homologous three-dimensional structure presented for binding first one and subsequently the other receptor subunit (in the case of dimerisation of the receptor).

As indicated above, when the polypeptide is a dimer, the two receptor-binding sites are structurally homologous. When the polypeptide is a trimer or higher, two or more of the receptor-binding sites may also be structurally homologous. In a preferred embodiment, all receptor-binding sites of a trimeric or higher polypeptide are structurally homologous.

Preferably, the receptor-binding sites are located in separate, symmetrical regions of the polypeptide. The term “separate” is intended to indicate that the receptor-binding sites are physically and structurally separated and thus not overlapping in the single-chain polypeptide so as to allow binding to both/all receptor-binding sites at a time (in contrast to the situation where the receptor-binding sites are located in the same region and where binding to one receptor-binding site blocks binding to a second receptor-binding site). The term “symmetrical” is intended to indicate that the receptor-binding sites are related by a pseudo symmetry axis, e.g. a pseudo two fold or three fold symmetry axis, which may coincide with a crystallographic symmetry axis.

In the present context, the terms “first receptor-binding site”, “second receptor-binding site”, etc. (or alternatively “receptor-binding site 1”, “receptor-binding site 2”, etc.) will be used as follows when referring to receptor-binding sites formed by amino acid residues located in two different monomeric units. Taking a trimeric single-chain polypeptide of the invention having monomeric subunits A, B and C as an example, the first receptor-binding site will be formed by monomers A and B, the second receptor-binding site by monomers B and C, and the third receptor-binding site by monomers C and A. Thus, a reference to e.g. “receptor-binding site 1” in this case refers to the binding site formed by the respective amino acid residues in the first two monomeric subunits of the polypeptide. Further, as used herein, the order of the monomeric subunits is given as is conventional in the art, i.e. for a polypeptide comprising e.g. first, second and third monomers, the first monomer is the N-terminal monomer and the third monomer is the C-terminal monomer.

In one preferred embodiment, the structurally homologous receptor-binding sites comprise the same amino acid residues on each of the monomers. Solely for illustration purposes, using as an example a homotrimeric polypeptide having three receptor-binding sites, the first receptor-binding site may consist of amino acid residues 12, 16, 17, 46, and 48 from monomer A and amino acid residues 64, 66, 83, 85, and 109 from monomer B, the second receptor-binding site of amino acid residues 12, 16, 17, 46, and 48 from monomer B and amino acid residues 64, 66, 83, 85, and 109 from monomer C, and the third receptor-binding-site of amino acid residues 12, 16, 17, 46, and 48 from monomer C and amino acid residues 64, 66, 83, 85, and 109 from monomer A.

It further appears from this example that the receptor-binding sites are “symmetrical”, in that, e.g., amino acid residues of monomer A taking part in formation of the first receptor-binding site are substantially identical to the amino acid residues of monomer B taking part in formation of the second receptor-binding site, etc.

The term “receptor” is understood to indicate a protein present on a cell surface which binds signalling molecules (i.e. ligands) as the first step in triggering the signal transduction cascade. Cell surface receptors are typically composed of different domains with different functions, such as an extracellular ligand-binding domain with which the signalling polypeptide interacts to initiate signal transduction, a transmembrane domain (or in some cases, several transmembrane domains) which anchors the receptor in the cell membrane, and an intracellular effector domain which generates a cellular signal in response to ligand binding (signal transduction). When the signalling polypeptide binds to the ligand-binding domain of a receptor subunit of a type requiring oligomerisation or other conformational changes to be activated, it subsequently binds to other closely located receptor subunits utilizing other receptor-binding sites (a process which is normally referred to as “receptor oligomerisation”, “receptor clustering” or “receptor aggregation”). This results in a conformational change of the receptor which leads to an interaction between the effector domain and one or more intracellular molecules (termed “effectors” herein) to effect a physiological change in the cell. According to the invention, modifications affecting a receptor-binding site may be carried out within the receptor-binding site itself, i.e. in a manner involving at least one of the amino acid residues forming part of the receptor-binding site, or may be carried out outside the receptor-binding site, but in a region of the polypeptide where such modification influences the folding and consequently the three-dimensional structure of the receptor-binding site or otherwise blocks access to the receptor-binding site so that the binding affinity of ligand binding to the receptor is significantly reduced and is insufficient to effect receptor activation.

Normally, the cell surface receptors of interest in the present context are composed of two or more subunits which may be identical, structurally homologous (as defined herein) or different. In particular, the receptors of interest are those which form oligomers, typically dimers, trimers, tetramers or higher oligomers, in response to the binding of a signalling polypeptide to a subunit of the receptor, resulting in recruitment of the other subunit(s) and binding of the signalling polypeptide to the ligand-binding domain(s) on the other subunit(s), and ultimately in activation of the receptor. The term “subunit” refers to individual receptor molecules required to form an active receptor. Normally, a dimeric receptor of this type will be activated by binding of a dimeric ligand, a trimeric receptor will be activated by binding of a trimeric ligand, etc.

Although attempts have been made to activate this type of receptor using the monomeric form of the associated signalling polypeptides, i.e. individual molecules, these attempts have failed through an inability to effect binding of the molecules to the receptor (see Pötgens et al. (1994) J. Biol. Chem. 269, 32879-32885; Claffey et al. (1995) Biochim. Biophys. Acta 1246, 1-9; Siemeister et al. (1998) Proc. Natl. Acad. Sci. USA 95, 4625-4629; and Fuh et al. (1998) J. Biol. Chem. 273, 11197-11204).

It should be noted that although the above-outlined receptor activation mechanism involving receptor oligomerisation is the present and generally accepted theory of how such receptors work (see, e.g., Heldin, supra), it will be clear from the above discussion that activation of such oligomeric receptors may not necessarily be dependent upon receptor oligomerisation as such, but rather may, at least in some cases, more generally involve conformational changes induced by binding of the oligomeric ligand (Chan et al. (2000) Science 288, 2351-2354; Siegel et al (2000) Science 288, 2354-2357). The present invention is therefore also intended to cover such situations. In other words, the present invention is equally applicable to receptors whose activation involves mechanisms other than simple oligomerisation, as long as the receptor activation is dependent on the binding of an oligomeric ligand to two or more receptor subunits.

In a particular embodiment, where the parent polypeptide is a trimer, the polypeptide of the invention comprises three monomer units (i.e. it is also a trimer). The trimer may be a homotrimer or a heterotrimer comprising three different monomers, or two identical monomers and one different monomer. When the polypeptide is a trimer, it may comprise two active receptor-binding sites and one inactive receptor-binding site, or one active receptor-binding site and two inactive receptor-binding sites.

While the inactive receptor-binding site is different from that of a corresponding native binding site, the active receptor-binding site may be unmodified, i.e. be constituted by the amino acid residues which are also found in the corresponding native binding site, or may be modified, e.g. to have an increased affinity towards a ligand binding domain of the receptor. In a preferred embodiment, the active binding site (or, in the case of a polypeptide with two active binding sites, one or both of the active binding sites) has such an increased binding affinity.

In a preferred embodiment, the polypeptide of the invention comprises at least one receptor-binding site with at least one modification that results in increased receptor-binding activity (i.e. increased affinity) of the modified receptor-binding site compared to a corresponding polypeptide without said modification. In this case, the active receptor-binding site will have an increased binding affinity compared to the parent polypeptide, thereby allowing an improved binding of the polypeptide to the receptor, and thus an improvement in the effect obtained by the inactive binding site. In the case of polypeptides comprising three or more monomers, it will be clear that one or more active binding sites may be modified to obtain increased binding affinity for the receptor. Thus, for a trimer having two active binding sites, one or both of these active binding sites may be modified for increased receptor-binding activity.

The different monomers of a heteromer may originate from the same parent oligomeric polypeptide (i.e. the parent oligmeric polypeptide is in itself a hetero-oligomeric polypeptide), or may contain monomers which originate from different polypeptides. For instance, one monomer may originate from one member of a given polypeptide family and a second monomer from another member of the same polypeptide family. In the latter case, it may be necessary to change amino acid residues of a monomer association domain required for assembly of the two or more monomers, e.g. as described in WO 96/40774. In the present context the term “association domain” is intended to indicate amino acid residues which line the contact points between monomers and which are essential for obtaining a proper assembly/conformation of an active single-chain oligomeric polypeptide of the invention. In the Materials and Methods section herein, a suitable method for determining amino acid residues of an association domain and of a receptor-binding site, respectively, are given.

Accordingly, the single-chain oligomeric polypeptide of the invention may be a heterooligomeric polypeptide wherein at least one of the monomers is modified in an association domain thereof so as to enable association of said monomer to one or more other monomers comprised in the heterooligomeric polypeptide in order to obtain an active single-chain oligomeric polypeptide. For instance, amino acid residues of an association domain of a first monomer to be modified may be replaced by amino acid residues of an associtiation domain of a second monomer to which the the first monomer is to associate.

In a preferred embodiment, the single-chain polypeptide of the invention is one wherein the inactive receptor-binding site is rendered inactive due to steric hindrance. For instance, steric hindrance is achieved when the receptor-binding site is blocked by a non-polypeptide moiety or is blocked by any part, preferably the side chain, of one or more amino acid residues which have been introduced (by insertion or more preferably by substitution) into one or more positions located in the receptor-binding site so that the relevant part of the amino acid residue(s) hinders binding to the ligand-binding domain of the receptor. For example, introduction of one or more bulky amino acid residues into the receptor-binding site may provide this effect.

Thus, in one embodiment, suitable modification of the polypeptide of the invention is advantageously effected by modification of the polypeptide in a least one position of a receptor-binding site. The polypeptide may be a variant (mutant form) of a native or wild-type ligand for a given receptor which furthermore is provided in single-chain form. Modification may be accomplished by suitable deletion, insertion, substitution or addition of one or more amino acid residues within the receptor-binding site. The modification should be of a type which essentially renders the receptor-binding site inactive as defined herein, but should, on the other hand, not be so extensive as to substantially alter the conformation of the other receptor-binding site(s) of the polypeptide and thereby render it/them incapable of binding to the ligand-binding domain of another receptor subunit. In addition to the above-described modification of one receptor-binding site, another receptor-binding site may be modified to become capable of more effectively binding to the ligand-binding domain of another receptor subunit as compared to the unmodified, native receptor-binding site.

The total number of amino acid residues to be altered in accordance with the present invention (as compared to the amino acid sequence of the receptor-binding site of the parent polypeptide) will typically not exceed 15. A receptor-binding site of the single-chain polypeptide thus preferably comprises an amino acid sequence which differs in 1-15 amino acid residues from the amino acid sequence of the corresponding receptor-binding site in the parent polypeptide in question, such as in 1-8 or 2-8 amino acid residues, e.g. in 1-5 or 2-5 amino acid residues. Thus, normally the polypeptide comprises an amino acid sequence which differs from the amino acid sequence of the receptor-binding site of the parent polypeptide in 1, 2, 3, 4, 5, 6, 7, 8, 9, 10, 11, 12, 13, 14 or 15 amino acid residues.

Inactivation of a Receptor-Binding Site by Amino Acid Residue Modification

Introduction, in particular by substitution, of an amino acid residue may be accomplished with any natural or synthetic amino acid residue, but is preferably one which significantly alters the properties of the receptor-binding site, such as substitution of a non-charged amino acid by a charged amino acid (e.g. arginine, lysine, glutamic acid or aspartic acid), substitution of a non-aromatic amino acid by an aromatic amino acid with a bulky side chain (e.g. phenylalanine, tryptophan, tyrosine), substitution of a non-hydrophobic amino acid by a hydrophobic amino acid (e.g. leucine, isoleucine, valine), substitution of a non-polar amino acid by a polar amino acid (glutamine or asparagine), substitution of a small amino acid (e.g. glycine, alanine, serine or threonine) by a bulkier amino acid (such as methionine or any of the amino acids mentioned above), or other suitable substitution. One type of substitution useful for the present purpose is substituting one or more amino acid residues in the receptor-binding site of a given polypeptide by an amino acid residue occupying an equivalent position in a homologous polypeptide (“homologous” in the sense that the polypeptides belong to the same family of polypeptides and exhibit a certain degree of sequence similarity, i.e. a sufficient sequence identity to allow alignment of the respective sequences). For instance, when the monomer to be modified is TNF-α, the amino acid substitution is one wherein one or more amino acid residues of the TNF-α receptor-binding site are replaced with the amino acid residues occupying equivalent positions in LT-α or LT-β. It is contemplated that such substitution does not impair the overall conformational structure of the single-chain oligomeric polypeptide, but is sufficient to contribute to inactivation of or as such to inactive the receptor-binding site thereof.

The terms “homology” and “identity” as used in connection with amino acid sequences are used in their conventional meanings. Amino acid sequence homology/identity is conveniently determined from aligned sequences (aligned by use of the algorithm CLUSTALW, version 1.74 (Thompson et al. (1994) CLUSTAL W: Improving the sensitivity of progressive multiple sequence alignment through sequence weighting, positions-specific gap penalties and weight matrix choice, Nucleic Acids Research, 22:4673-4680) using default parameters) or provided from the PFAM families database version 4.0 (http://pfam.wustl.edu/) (Nucleic Acids Res Jan. 1, 1999; 27 (1):260-2) by use of GENEDOC version 2.5 (Nicholas, K. B., Nicholas H. B. Jr., and Deerfield, D. W. II. 1997 GeneDoc: Analysis and Visualization of Genetic Variation, EMBNEW.NEWS 4:14; Nicholas, K. B. and Nicholas H. B. Jr. 1997 GeneDoc: Analysis and Visualization of Genetic Variation).

The term “polypeptide family” is used in its conventional meaning, i.e. to indicate a group of polypeptides which are related to each other by having an amino acid sequence which exhibits a sufficient degree of identity to allow alignment of the sequences. Polypeptide families are available, e.g. from the PFAM families database, version 4.0, or the PROSITE data base (Hofmann et al., The PROSITE database, its status in 1999 Nucleic Acids Res. 27:215-219(1999)) or may be prepared by use of a suitable computer program such as CLUSTALW version 1.74. Furthermore, the protein sequence family may be provided from recursive searches in protein sequence databases like SWISS-PROT or TrEMBL (Bairoch A., Apweiler R. The SWISS-PROT protein sequence data bank and its supplement TrEMBL in 1999 Nucleic Acids Res. 27:49-54 (1999)) using well established sequence search/comparison algorithms like FASTA (Pearson W. R. and Lipman D. J. (1981) Proc. Natl. Acad. Sci. U.S.A. 85. 2444-2448), BLAST (Altshul, S. F. et al. (1997) Nucleic Acids Res. 25. 3389-3402), PSI-BLAST (Altschul et al. (1997), “Gapped BLAST and PSI-BLAST: A new generation of protein database search programs”, Nucleic Acids Res. 25:3389-3402.) or from searches in nucleotide sequence data bases like EMBL (Stoesser et al., Nucleic Acids Research, 1999, 27(1):18-24) or GENEBANK (Benson et al., Nucleic Acids Res 1999, 27(1):12-17) using equally well established, search algorithms. An overview of these methods can be found in Trends Guide to Bioinformatics (1998), Elsevier Science.

A model structure may easily be constructed by the skilled person on the basis of the known three-dimensional structure of another member of the polypeptide family to which the polypeptide of interest belongs. In order to be able to construct a model structure it is normally desirable that the polypeptide of interest displays at least 30% sequence identity with the polypeptide with the known three-dimensional structure. The model structure may be constructed using any suitable software known in the art, for example the software Modeller (Andrej Sali, Roberto Sánchez, Azat Badretdinov, András Fiser, and Eric Feyfant, The Rockefeller University, 1230 York Avenue, New York, N.Y. 10021-6399, USA) or the software WHAT IF: A molecular modeling and drug design program (G. Vriend, J. Mol. Graph. (1990) 8, 52-56).

The term “equivalent position” is intended to indicate a position in the amino acid sequence of a given polypeptide which is homologous (i.e. corresponding in position in either primary or tertiary structure) to a position in the amino acid sequence of another polypeptide belonging to the same polypeptide sequence family. Where possible, the “equivalent position” is conveniently determined on the basis of an alignment of members of the polypeptide sequence family in question or alternatively on the basis of superimposed three-dimensional structures, e.g. using the program CLUSTALW version 1.74 using default parameters (Thompson et al., 1994, CLUSTAL W: Improving the sensitivity of progressive multiple sequence alignment through sequence weighting, position-specific gap penalties and weight matrix choice, Nucleic Acids Research, 22:4673-4680).

Inactivation of a Receptor-Binding Site by Introduction of an Attachment Group for a Non-Polypeptide Moiety

In accordance with the invention, as an alternative to or for further improvement of the above described amino acid residue modification, a receptor-binding site may be blocked by a non-polypeptide moiety. Said non-polypeptide moiety is conjugated or otherwise coupled to the single-chain polypeptide through an attachment group of an amino acid residue which is located so as to allow the conjugated non-polypeptide moiety to block the receptor-binding site. The non-polypeptide moiety may for instance be a polymer molecule, a carbohydrate (or oligosaccharide) molecule, a lipophilic molecule or an organic derivatizing agent. The “polymer molecule” is a molecule formed by covalent linkage of two or more monomers, wherein none of the monomers is an amino acid residue, except where the polymer is human albumin or another abundant plasma protein. The term “polymer” may be used interchangeably with the term “polymer molecule”. The term is intended to cover carbohydrate molecules, although, normally, the term is not intended to cover the type of carbohydrate molecule which is attached to the polypeptide by in vivo N- or O-glycosylation (as further described below). Except where the number of non-polypeptide moieties, e.g. polymer molecules, is expressly indicated, every reference to e.g. a “polymer” or “polymer molecule” contained in a single-chain polypeptide of the invention or otherwise used in the present context shall be understood to be a reference to one or more such polymer molecule(s).

The term “attachment group” is intended to indicate a functional group of the polypeptide, in particular of an amino acid residue thereof or an oligosaccharide moiety, capable of attaching a non-peptide moiety such as a polymer molecule, a lipophilic molecule or an organic derivatizing agent. Useful attachment groups and their matching non-peptide moieties are apparent from the table below.

| Attachment | | Example of non- | Conjugation method/- | |
|---|---|---|---|---|
| group | Amino acid | peptide moiety | Activated PEG | Reference |
| —NH₂ | N-terminal, | Polymer, e.g. | mPEG-SPA | Shearwater Inc. |
| | Lys | PEG, with | Tresylated mPEG | Delgado et al, |
| | | amide or imine | | critical reviews in |
| | | group | | Therapeutic Drug |
| | | | | Carrier Systems |
| | | | | 9(3, 4): 249-304 |
| | | | | (1992) |
| —COOH | C-terminal, | Polymer, e.g. | mPEG-Hz | Shearwater Inc. |
| | Asp, Glu | PEG, with ester | | |
| | | or amide group | | |
| | | Oligosaccharide | In vitro coupling | |
| | | moiety | | |
| —SH | Cys | Polymer, e.g. | PEG-vinylsulphone | Shearwater Inc. |
| | | PEG, with | PEG-maleimide | Delgado et al., |
| | | disulfide, | | critical reviews in |
| | | maleimide or | | Therapeutic Drug |
| | | vinyl sulfone | | Carrier Systems |
| | | group | In vitro coupling | 9(3, 4): 249-304 |
| | | Oligosacchande | | (1992) |
| | | moiety | | |
| —OH | Ser, Thr, | Oligosaccharide | In vivo O-linked | |
| | OH—, Lys | moiety | glycosylation | |
| | | PEG with ester, | | |
| | | ether, | | |
| | | carbamate, | | |
| | | carbonate | | |
| —CONH₂ | Asn as part | Oligosaceharide | In vivo N- | |
| | of an N- | moiety | glycosylation | |
| | glycosyla- | Polymer, e.g. | | |
| | tion site | PEG | | |
| Aromatic | Phe, Tyr, | Oligosaccharide | In vitro coupling | |
| residue | Trp | moiety | | |
| —CONH₂ | Gln | Oligosaccharide | In vitro coupling | Yan and Wold, |
| | | moiety | | Biochemistry, |
| | | | | 1984, Jul 31; |
| | | | | 23(16): 3759-65 |
| Aldehyde | Oxidized | Polymer, e.g. | PEGylation | Andresz et al., |
| Ketone | oligo- | PEG, | | 1978, Makromol. |
| | saccharide | PEG-hydrazide | | Chem. 179: 301, |
| | | | | WO 92/16555, WO |
| | | | | 00/23114 |
| Guanidino | Arg | Oligosaccharide | In vitro coupling | Lundblad and |
| | | moiety | | Noyes, Chimical |
| | | | | Reagents for |
| | | | | Protein |
| | | | | Modification, CRC |
| | | | | Press Inc. Boca |
| | | | | Raton, FI |
| Imidazole | His | Oligosaccharide | In vitro coupling | As for guanidine |
| ring | | moiety | | |

For in vivo N-glycosylation, the term “attachment group” is used in an unconventional way to indicate the amino acid residues constituting an N-glycosylation site (with the sequence N-X′-S/T/C-X″, wherein X′ is any amino acid residue except proline, X″ any amino acid residue which may or may not be identical to X′ and which preferably is different from proline, N is asparagine, and S/T/C is either serine, threonine or cysteine, preferably serine or threonine, and most preferably threonine). Although the asparagine residue of the N-glycosylation site is where the oligosaccharide moiety is attached during glycosylation, such attachment cannot be achieved unless the other amino acid residues of the N-glycosylation site are present. Accordingly, when the non-peptide moiety is an oligosaccharide moiety and the conjugation is to be achieved by N-glycosylation, the term “amino acid residue comprising an attachment group for the non-peptide moiety” as used in connection with alterations of the amino acid sequence of the polypeptide of interest is to be understood as meaning that one or more amino acid residues constituting an N-glycosylation site are to be altered in such a manner that either a functional N-glycosylation site is introduced into the amino acid sequence or removed from said sequence.

The polymer molecule to be coupled to the polypeptide may be any suitable polymer molecule, such as a natural or synthetic homo-polymer or hetero-polymer, typically with a molecular weight in the range of 300-100,000 Da, such as 300-20,000 Da, more preferably in the range of 500-10,000 Da, even more preferably in the range of 500-5000 Da. Examples of homo-polymers include a polyol (i.e. poly-OH), a polyamine (i.e. poly-NH₂) and a polycarboxylic acid (i.e. poly-COOH). A hetero-polymer is a polymer which comprises different coupling groups, such as a hydroxyl group and an amine group.

Examples of suitable polymer molecules include polymer molecules selected from the group consisting of polyalkylene oxide (PAO), including polyalkylene glycol (PAG), such as polyethylene glycol (PEG) and polypropylene glycol (PPG), branched PEGs, poly-vinyl alcohol (PVA), poly-carboxylate, poly-(vinylpyrolidone), polyethylene-co-maleic acid anhydride, polystyrene-co-maleic acid anhydride, dextran, including carboxymethyl-dextran, or any other biopolymer suitable for blocking a receptor-binding site, and optionally for reducing immunogenicity and/or increasing functional in vivo half-life and/or serum half-life. Another example of a polymer molecule is human albumin or another abundant plasma protein. Generally, polyalkylene glycol-derived polymers are biocompatible, non-toxic, non-antigenic, non-immunogenic, have various water solubility properties, and are easily excreted from living organisms.

PEG is the preferred polymer molecule, since it has only few reactive groups capable of cross-linking compared, e.g., to polysaccharides such as dextran, and the like. In particular, monofunctional PEG, e.g. methoxypolyethylene glycol (mPEG), is of interest since its coupling chemistry is relatively simple (only one reactive group is available for conjugating with attachment groups on the polypeptide). Consequently, the risk of cross-linking is eliminated, the resulting polypeptide conjugates are more homogeneous and the reaction of the polymer molecules with the polypeptide is easier to control.

To effect covalent attachment of the polymer molecule(s) to the polypeptide, the hydroxyl end groups of the polymer molecule must be provided in activated form, i.e. with reactive functional groups. Suitable activated polymer molecules are commercially available, e.g. from Shearwater Polymers, Inc., Huntsville, Ala., USA. Alternatively, the polymer molecules can be activated by conventional methods known in the art, e.g. as disclosed in WO 90/13540. Specific examples of activated linear or branched polymer molecules for use in the present invention are described in the Shearwater Polymers, Inc. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g. SPA-PEG, SSPA-PEG, SBA-PEG, SS-PEG, SSA-PEG, SC-PEG, SG-PEG, and SCM-PEG), and NOR-PEG), BTC-PEG, EPOX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. No. 5,932,462 and U.S. Pat. No. 5,643,575, both of which are incorporated herein by reference. Furthermore, the following publications, incorporated herein by reference, disclose useful polymer molecules and/or PEGylation chemistries: U.S. Pat. No. 5,824,778, U.S. Pat. No. 5,476,653, WO 97/32607, EP 229,108, EP 402,378, U.S. Pat. No. 4,902,502, U.S. Pat. No. 5,281,698, U.S. Pat. No. 5,122,614, U.S. Pat. No. 5,219,564, WO 92/16555, WO 94/04193, WO 94/14758, WO 94/17039, WO 94/18247, WO 94/28024, WO 95/00162, WO 95/11924, WO 95/13090, WO 95/33490, WO 96/00080, WO 97/18832, WO 98/41562, WO 98/48837, WO 99/32134, WO 99/32139, WO,99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. No. 5,473,034, U.S. Pat. No. 5,516,673, EP 605 963, U.S. Pat. No. 5,382,657, EP 510 356, EP 400 472, EP 183 503 and EP 154 316.

The conjugation of the polypeptide and the activated polymer molecules is conducted by use of any conventional method, e.g. as described in the following references (which also describe suitable methods for activation of polymer molecules): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). The skilled person will be aware that the activation method and/or conjugation chemistry to be used depends on the attachment group(s) of the polypeptide (examples of which are given further above), as well as the functional groups of the polymer (e.g. being amine, hydroxyl, carboxyl, aldehyde, sulfydryl, succinimidyl, maleimide, vinysulfone or haloacetate). The PEGylation may be directed towards conjugation to all available attachment groups on the polypeptide (i.e. such attachment groups that are exposed at the surface of the polypeptide) or may be directed towards one or more specific attachment groups, e.g. the N-terminal amino group (U.S. Pat. No. 5,985,265). Furthermore, the conjugation may be achieved in one step or in a stepwise manner (e.g. as described in WO 99/55377).

It will be understood that the PEGylation will be designed so as to produce the optimal molecule with respect to the number of PEG molecules attached, the size and form of such molecules (e.g. whether they are linear or branched), and the attachment site(s) in the polypeptide. The molecular weight of the polymer to be used may e.g. be chosen on the basis of the desired effect to be achieved, for instance taking into consideration whether the primary purpose of the conjugation is to block a receptor-binding site, or whether there is an additional or alternative purpose, e.g. to reduce renal clearance by means of a high molecular weight or to provide epitope shielding in order to reduce immunogenicity. For example, for reduced renal clearance it is usually desirable to conjugate as few high Mw polymer molecules as possible to obtain the desired molecular weight, while epitope shielding may be obtained by use of a sufficiently high number of low molecular weight polymer molecules (e.g. with a molecular weight of about 5000 Da), for instance 2-8, such as 3-6 such polymer molecules.

Normally, the polymer conjugation is performed under conditions aiming at reacting all available polymer attachment groups with polymer molecules. Typically, the molar ratio of activated polymer molecules to polypeptide is up to about 500-1, in particular 200-1, preferably 100-1, such as 10-1 or 5-1 in order to obtain optimal reaction.

It is also contemplated according to the invention to couple the polymer molecules to the polypeptide through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. Ed., 24, 375-378.

Subsequent to the conjugation, residual activated polymer molecules are blocked according to methods known in the art, e.g. by addition of primary amine to the reaction mixture, and the resulting inactivated polymer molecules are removed by a suitable method.

Specific PEGylation strategies include, for example: 1) a single PEG molecule attached to the N-terminal of the polypeptide and no other PEG molecules, e.g. a linear or branched PEG molecule with a molecular weight of at least about 20 kDa, the-polypeptide optionally further comprising one or more oligosaccharide moieties attached to an N-linked or O-linked glycosylation site of the polypeptide or carbohydrate moieties attached by in vitro glycosylation; 2) a PEG molecule attached to each of the lysine residues in the polypeptide available for PEGylation, e.g. a linear or branched PEG molecule with a molecular weight of about 5 kDa; and 3) a PEG molecule attached to each of the lysine residues in the polypeptide available for PEGylation, and in addition to the N-terminal amino acid residue of the polypeptide.

The conjugation to an oligosaccharide moiety may take place in vivo or in vitro. In order to achieve in vivo glycosylation of a single-chain polypeptide comprising one or more glycosylation sites, the nucleotide sequence encoding the polypeptide must be inserted in a glycosylating, eucaryotic expression host. The expression host cell may be selected from fungal (filamentous fungal or yeast), insect or animal cells or from transgenic plant cells. In one embodiment the host cell is a mammalian cell, such as a CHO cell, BHK or HEK cell, or an insect cell, such as an SF9 cell, or a yeast cell, e.g. Saccharomyces cerevisiae or Pichia pastoris, or any of the host cells mentioned hereinafter.

Covalent in vitro coupling of glycosides (such as dextran) to amino acid residues of the single-chain polypeptide may also be used, e.g. as described in WO 87/05330 and in Aplin etl al., CRC Crit Rev. Biochem., pp. 259-306, 1981.

Furthermore, the in vitro coupling of oligosaccharide moieties or PEG to protein- and peptide-bound Gln-residues can be carried out by transglutaminases (TGases). Transglutaminases catalyse the transfer of donor amine-groups to protein- and peptide-bound Gln-residues in a so-called cross-linking reaction. The donor-amine groups can be protein- or peptide-bound e.g. as the e-amino-group in Lys-residues or can be part of a small or large organic molecule. An example of a small organic molecule functioning as an amino-donor in TGase-catalysed cross-linking is putrescine (1,4-diaminobutane). An example of a larger organic molecule functioning as an amino-donor in TGase-catalysed cross-linking is an amine-containing PEG (Sato et al., Biochemistry 35, 13072-13080). TGases, in general, are highly specific enzymes, and not every Gln-residue exposed on the surface of a protein is accessible to TGase-catalysed cross-linking to amino-containing substances. On the contrary, only few Gin-residues function naturally as TGase substrates, but the exact parameters governing which Gln-residues are good TGase substrates remain unknown. Thus, in order to render a protein susceptible to TGase-catalysed cross-linking reactions it is often a prerequisite at convenient positions to add stretches of amino acid sequence known to function very well as TGase substrates. Several amino acid sequences are known to be or to contain excellent natural TGase substrates, e.g. substance P, elafin, fibrinogen, fibronectin, α₂-plasmin inhibitor, α-caseins, and β-caseins.

If an amino acid residue comprising an attachment group for the non-polypeptide moiety in question is located in the vicinity of the receptor-binding site, modification may simply be achieved by conjugation of the non-polypeptide moiety of choice to the attachment group in question. Subsequently, the resulting conjugated single-chain polypeptide is tested for its capability to bind to a ligand-binding domain of the receptor, and incapability of effecting signal transduction, e.g. as described in the section hereinbelow entitled “Assay”. For single chain polypeptides of the invention having two or more identical receptor-binding sites (or having an amino acid residue comprising an attachment group for the non-polypeptide moiety in question located in the vicinity of one or more further receptor-binding sites) it may be necessary to remove such amino acid residue(s) from the second or more receptor-binding site(s), preferably by conservative amino acid substitution, to ensure that the non-polypeptide moiety is conjugated only to the intended amino acid residue, and not to amino acid residues located in the vicinity of other receptor-binding site(s) for which no reduction of receptor-binding capability is intended. Alternatively, an amino acid residue comprising an attachment group for the non-polypeptide moiety of choice may be introduced, preferably by substitution, within the receptor-binding site to be modified, either by use of site-directed mutagenesis or by random mutagenesis. When site-directed mutagenesis is used, the actual position(s) to be modified are conveniently selected on the basis of an analysis of the three-dimensional structure of the receptor-binding site to be modified. When random mutagenesis is used, it is normally limited to amino acid residues of the receptor-binding site to be modified. The site-directed or random mutagenesis is-normally accompanied by a suitable screening of the resulting polypeptide variants, e.g. as described in the section entitled “Assay” herein below. Preferably, the variants resulting from site-directed or random mutagenesis are conjugated to the non-polypeptide moiety of choice prior to screening.

When an amino acid residue comprising an attachment group is to be introduced into a receptor-binding site or in the vicinity thereof so that a non-polypeptide moiety conjugated to said amino acid residue inactivates the receptor-binding site, it is usually sufficient that such amino acid residue is introduced in only one of the monomers contributing to the receptor-binding site. Furthermore, when an amino acid residue comprising an attachment group is to be introduced into or in the vicinity of the receptor-binding site this should preferably be done so that the attachment group for the non-polypeptide moiety is exposed at the surface of the polypeptide and thereby rendered accessible for conjugation to the non-polypeptide, moiety. The latter may be evaluated in a model or 3D structure of the single-chain polypeptide or of the receptot-binding site.

Preferably, when the non-polypeptide moiety is a polymer molecule, the amino acid residue comprising an attachment group for the non-polypeptide moiety is a cysteine residue, since, in general, it is possible to obtain a selective conjugation to such residue.

Furthermore, as explained above, in order to avoid conjugation to the non-polypeptide moiety of choice in regions of the single-chain polypeptide where such conjugation is not desirable, the single-chain polypeptide may be further modified so as to have removed, preferably by substitution, even more preferably by conservative substitution, one or more amino acid residues comprising an-attachment group for said non-polypeptide moiety. For instance, it may be important to remove such amino acid residue, if it is present in the intact receptor-binding site of the single-chain polypeptide.

When the single-chain polypeptide of the invention is a trimer comprising first, second and third monomers, the third monomer may be modified in at least one position within each of two receptor-binding sites thereof, and one of the first and second monomers may additionally be modified in at least one other position within these same receptor-binding sites such that the polypeptide comprises one intact receptor-binding site capable of binding to a ligand-binding domain of the cellular receptor and two inactive receptor-binding sites. Analogously, to modify only one receptor-binding site, the third monomer may be modified in at least one position within a single receptor-binding site, and one of the first or second monomers is modified in at least one other position within said receptor-binding site, the remaining receptor-binding sites being left intact.

In accordance with this invention, it is also contemplated to improve binding of the, polypeptide to a receptor through the intact, active receptor-binding site by substitution or insertion of one or more amino acid residues in said receptor-binding site so as to obtain a stronger binding affinity to said receptor. It is at present assumed that suitable amino acid substitutions may be conservative substitutions. Examples of such conservative substitutions are substitution within the group of basic amino acids (such as arginine, lysine or histidine), acidic amino acids (such as glutamic acid or aspartic acid), polar amino acids (such as glutamine or asparagine), hydrophobic amino acids (such as leucine, isoleucine or valine), aromatic amino acids (such as phenylalanine, tryptophan or tyrosine) and small amino acids (such as glycine, alanine, serine or threonine).

Preferred substitutions in the present invention are listed in Table 1.

TABLE 1

| Amino acid | Substituted by amino acid |
|---|---|
| A | D, N, E, Q, H, Y, R, K |
| D | G, A, S, T, N, Q, H, Y, R, K |
| E | G, A, S, T, N, Q, H, Y, R, K |
| F | G, A, S, T, D, N, E, Q, R, K |
| G | D, N, E, Q, H, Y, R, K |
| H | G, A, S, T, D, E, R, K |
| I | G, A, S, T, D, N, E, Q, H, Y, R, K |
| K | G, A, S, T, D, N, E, Q, Y |
| L | G, A, S, T, D, N, E, Q, H, Y, R, K |
| M | G, A, S, T, D, N, E, Q, H, Y, R, K |
| N | G, A, S, T, D, E, Y, R, K |
| P | G, A, S, T, D, N, E, Q, H, Y, R, K |
| Q | G, A, S, T, D, E, Y, R, K |
| R | G, A, S, T, D, N, E, Q, H, Y |
| S | D, E, H, Y, R, K |
| T | D, E, H, Y, R, K |
| V | G, A, S, T, D, N, E, Q, H, Y, R, K |
| W | G, A, S, T, D, N, E, Q, H, Y, R, K |
| Y | G, A, S, T, D, E, R, K |

For some of the native parent polypeptides of interest to the present invention, the receptor-binding site has been identified (see for instance, Muller et al. (1997) Proc. Natl. Acad. Sci. USA 94, 7192-7197; Siemeister et al. (1998) Proc. Natl. Acad. Sci. USA 95, 4625-4629; Fuh et al. (1998) J. Biol. Chem. 273, 11197-11204; Wiesmann et al. (1997) Cell 91, 695-704; and Terman et al. (1992) Biochem Biophys. Res. Commun. 187 1579-1586). However, in case the receptor-binding site is not known, it may be identified by homology search to known receptor-binding sites. Alternatively, amino acid residues essential to the binding and signal transducing capabilities of the present polypeptide may be identified according to procedures known in the art such as site-directed mutagenesis or alanine-scanning mutagenesis (Cunningham and Wells, Science 244, 1989, pp. 1081-1085). According to the latter technique, mutations are introduced at every amino acid position of the polypeptide, and the resulting mutant molecules are tested for the relevant biological activity, in this case receptor-binding capability, to identify amino acid residues that are critical to that particular activity (see also Hilton et al., J. Biol. Chem. 271, 1996, pp. 4699-4708). Sites of ligand-receptor interaction may also be determined by analysis of the three-dimensional structure as determined by such techniques as nuclear magnetic resonance or X-ray crystallography, in conjunction with mutation of putative receptor-binding site amino acids. See for example de Vos et al., Science 255, 1992, pp. 306-312; Smith et al., J. Mol. Biol. 224, 1992, pp. 899-904; Wlodaver et al., FEBS Lett. 309, 1992, pp. 59-64.

Peptide Linker

The monomers composing the polypeptide may be linked by a peptide bond, or may be connected by a suitable linker peptide. Choosing a suitable linker for a specific case where two polypeptide chains are to be connected depends on various parameters, e.g. the nature of the two polypeptide chains (e.g. whether they naturally form a dimer or not), the distance between the N- and C-termini to be connected if known from three-dimensional structure determination, and/or the stability of the linker towards proteolysis and oxidation. Furthermore, the linker may contain amino acid residues that provide flexibility. The linker peptide may therefore predominantly include the following amino acid residues: Gly, Ser, Ala or Thr. The linker peptide should have a length which is adequate to link two monomers in such a way that they assume the correct conformation relative to one another so that they retain the desired activity as antagonists of a given receptor. A suitable length for this purpose is a length of at least one and not more than about 30 amino acid residues, such as a sequence of about 5-20 amino acid residues, in particular about 10-15 amino acid residues. Likewise, the amino acid residues selected for inclusion in the linker peptide should exhibit properties that do not interfere significantly with the activity of the polypeptide. Thus, the linker peptide should on the whole not exhibit a charge which would be inconsistent with the activity of the polypeptide, or interfere with internal folding, or form bonds or other interactions with amino acid residues in one or more of the monomers which would seriously impede the binding of the polypeptide to the ligand-binding domain of the receptor in question.

The use of naturally occurring as well as artificial polypeptide linkers to connect polypeptides into novel linked fusion polypeptides is well known in the literature (Hallewell et al. (1989), J. Biol. Chem. 264, 5260-5268; Alfthan et al. (1995), Protein Eng. 8, 725-731; Robinson & Sauer (1996), Biochemistry 35, 109-116; Khandekar et al. (1997), J. Biol. Chem. 272, 32190-32197; Fares et al. (1998), Endocrinology 139, 2459-2464; Smallshaw et al. (1999), Protein Eng. 12, 623-630; U.S. Pat. No. 5,856,456).

One example where the use of polypeptide linkers is widespread is for production of single-chain antibodies where the variable regions of a light chain (V_{L}) and a heavy chain (V_{H}) are joined through an artificial linker, and a large number of publications exist within this particular field. A widely used linker polypeptide is a 15mer consisting of three repeats of a Gly-Gly-Gly-Gly-Ser amino acid sequence ((Gly₄Ser)₃). Other linkers have been used and phage display technology as well as selective infective phage technology has been used to diversify and select appropriate linker sequences (Tang et al. (1996), J. Biol. Chem. 271, 15682-15686; Hennecke et al. (1998), Protein Eng. 11, 405-410). Polypeptide linkers have been used to connect individual chains in hetero- and homo-dimeric proteins such as the T-cell receptor, the lambda Cro repressor, the P22 phage Arc repressor, IL-12, TSH, FSH, IL-5, and interferon-γ. Polypeptide linkers have also been used to create fusion polypeptides. Various linkers have been used and in the case of the Arc repressor phage display has been used to optimize the linker length and composition for increased stability of the single-chain protein (Robinson and Sauer (1998), Proc. Natl. Acad. Sci. USA 95, 5929-5934).

It is also possible to identify suitable linkers by screening databases of known three-dimensional structures for naturally occurring motifs that can bridge the gap between the two polypeptide chains to be connected, given knowledge of the three-dimensional structure of a complex of the two polypeptide chains. Another way of obtaining a suitable linker is by optimizing a simple linker-e.g. ((Gly₄Ser)ₙ)-through random mutagenesis.

Another type of linker of particular interest in the present invention is an intein, i.e. a peptide stretch which is expressed with the single-chain polypeptide, but removed post-translationally by protein splicing. The use of inteins is reviewed by F. S. Gimble in Chemistry and Biology, 1998, Vol 5, No. 10 pp. 251-256.

The monomeric polypeptides to be linked according to the invention may be provided in truncated form, e.g. having 1-10, such as 1-5 of the amino acid residues of either the N- or C-terminal deleted relative to the corresponding wild-type polypeptide, and the truncated monomeric polypeptides may be fused to each other directly or linked through a peptide linker as discussed above. This latter phenomenon may be illustrated for a single-chain trimeric polypeptide of the invention comprising three TNF-α monomers. From the available TNF-α structure information (see the discussion of TNF-α below) it is apparent that the first 5 N-terminal amino acid residues could not be located in the structure (indicating large flexibility) and further that amino acid residue 9 from the N-terminal is closest to the C-terminal of the following monomeric polypeptide. Accordingly, when two TNF-α monomers are to be linked, the first of these may be deleted of up to 6 N-terminal amino acid residues, such as 5, 4, 3 or 2 N-terminal amino acid residues or a single amino acid residue. The second, and third monomer may be equally truncated. The resulting truncated polypeptide(s) may be linked to other monomeric polypeptide constituents through a peptide linker, or may be directly fused to one another.

The types of cellular receptors of relevance for the present invention are those that are activated by oligomerisation or other conformational change following ligand binding and they belong to a wide variety of classes of interest for a great many therapeutic purposes. Thus, for instance, the cellular receptor may be selected from the group consisting of cytokine receptors, protein-tyrosine kinase receptors, death domain receptors, including the TNF receptor family, and serine-threonine kinase receptors.

Cytokine receptors include receptors for many interleukins, colony-stimulating factors and certain other factors and hormones. Cytokine receptors may be divided into class I and class II receptors according to the presence of different conserved motifs. Ligand binding induces oligomerisation of the receptors and this allows interaction and activation of cytoplasmic protein-tyrosine kinases that are associated with the intracellular domain of the receptors. Most of the class I receptors undergo hetero-oligomerisation after ligand binding. In many cases, the ligand-binding subunit(s) form signalling complexes with signal-transducing molecules which are structurally related to cytokine receptors but are in themselves unable to bind ligands. For instance, interleukin-3 (IL-3), IL-5 and granulocyte/macrophage colony simulating factor (GM-CSF) bind to specific α-subunit receptors, after which the complexes interact with a common β-subunit required for high affinity ligand binding and signal transduction. Similar situations where cytokine-specific receptor α-subunits share a common subunit essential for signal transduction exist in at least two other groups of cytokines. IL-2, IL-4, IL-7, and IL-9 are examples from one group while IL-6, IL-11, leukaemia inhibitory factor, and oncostatin M are examples from the other group. A common feature for all the receptors mentioned is that activation occurs through hetero-dimerisation. Other cytokine receptors are activated by homo-dimerisation, such as the receptors for erythropoietin, prolactin, and granulocyte colony stimulating factor (G-CSF). Most of the aforementioned cytokines are monomeric. Examples of oligomreric cytokines suitable for modification for the present purpose are TNF-α, IFN-γ, lymphotoxin-α, lymphotoxin-β, FasL, CD40L, CD30L, CD27L, Ox40L, 4-1BBL, RANKL, TRAIL, TWEAK, LIGHT, TRANCE, APRIL, THANK and TALL-1, IL-10 and IL-16. One preferred cytokine in accordance with the present invention is TNF-α, which is discussed in greater detail below.

An example of another cytokine of interest is osteoprotegerin (OPG), which belongs to the TNF receptor family, and its ligand OPGL (identical to RANKL), which is also a ligand for RANK. OPG, RANK and OPGL play key roles in bone formation and destruction, respectively, and OPG has been suggested for the treatment of different osteodegenerative diseases, in particular osteoporosis. Wild-type OPG may, however, be a less than optimal treatment as it may serve as a carrier for OPGL and thus enable increased OPGL activity. Furthermore, treatment with OPG may elicit development of antibodies that may stimulate RANK.

Studies on OPG strongly suggest that OPG blocks the differentiation of osteoclasts, which are the principal if not sole bone-resorbing cell type. OPG has therefore been suggested as a humoral regulator of bone resorption. OPGL on the other hand has been found to activate mature osteoclasts and to modulate osteoclast formation from bone marrow precursors in the presence of CSF-1. Soluble OPGL has in addition been shown to be a potent inducer of bone resorption in vivo (Lacey et al. (1998), Cell 93: 165-176). The available data thus suggests a model in which OPGL and OPG act as positive and negative regulators of osteoclast development, respectively, i.e. OPGL promotes bone resorption while OPG inhibits bone resorption. Down-regulation of osteoclast differentiation/maturation/formation and osteoclast activation through neutralisation of OPGL using the single-chain antagonist technology of the present invention could therefore provide a safe and effective treatment for osteoporosis that is capable of providing an adequate blockade of OPGL activity.

OPGL is synthesised as a type II transmembrane protein consisting of 317 amino acid residues (human) or 316 amino acid residues (murine). Alignment of the two amino acid sequences show that identical amino acid residues are found at 87% of the homologous positions. The amino acid sequence of OPGL contains a short cytoplasmic domain in the N-terminus followed by the putative transmembrane region between amino acid residues 49 and 69. Based on the homology to TNF-α, the extracellular part of OPGL is suggested to be comprised by two domains: a stalk region extending from amino acid residue 70 to 157, and the active ligand moiety extending from amino acid residue 158 to the C-terminus. The amino acid sequence of OPGL is disclosed in Lacey et al. 1998 (supra).

OGP comprises 401 amino acid residues and is synthesized as a monomer of approximately 55 kDa. The mouse and human OGP proteins are approximately 85 percent and 94 percent identical with the rat protein, respectively (COPE: Cytokines Online Pathfinder Encyclopedia, (http://www.copewithcytokines.de/cope.cgi). The amino acid sequence of OPG is disclosed in Simonet et al. (1997) Cell 89: 309-319.

OPGL exists as a trimer in solution and it forms 3:3 complexes when incubated with OPG. One embodiment of the invention thus relates to a single-chain OPGL antagonist comprising three monomeric units derived from OPGL, wherein one or more of the monomers are modified so that at least one receptor-binding site, and preferentially two receptor-binding sites, of the single-chain trimeric polypeptide is/are rendered inactive. The resulting ligand is thus able to bind to RANK and prevents binding of OPGL (RANKL) but is unable to activate RANK.

Protein-tyrosine kinase receptors form another large class of receptors which are activated by oligomerisation and which in some cases may be antagonised by polypeptides of the present invention. Several of the native ligands for protein-tyrosine kinase receptors are growth factors. Examples of oligomeric growth factors suitable for modification in accordance with the principles of the present invention include vascular endothelial growth factor (VEGF), VEGF-B, VEGF-C, VEGF-D, platelet-derived growth factor (PDGF), placental growth factor,(PlGF), transforming growth factor β1 (TGF-β1), TGF-β2, TGF-β3, TGF-β4, bone morphogenic protein 2 (BMP-2), BMP-3, BMP-4, BMP-5, BMP-6, BMP-7, BMP-8, growth differentiating factor 1 (GDF-1), GDF-5, GDF-8 (myostatin), GDF-10, Muellerian inhibiting factor, inhibin A, inhibin B, activin A and activin AB. As is the case with cytokine receptors, the receptors may for instance be homodimerised or heterodimerised. For example, different isoforms of PDGF induce different dimeric forms of the PDGF receptor.

The present polypeptide is normally provided in isolated form, i.e. it is present in a condition other than its native environment. In a preferred form, the isolated polypeptide is substantially free of other proteins, such as proteins produced by a given host cell. It is preferred to provide the polypeptide in a highly purified form, i.e. more than 90% pure, more preferably more than 95% pure, such as about 99% pure, as determined by for instance reducing and non-reducing SDS-PAGE using Coomassie Brilliant Blue or silver staining. For therapeutic purposes, the polypeptide should be at least 95% pure.

One problem that may be encountered when using a single-chain oligomeric polypeptide of the invention is that the polypeptide or parts thereof, e.g. a peptide linker used in the construction of the polypeptide, may be recognized as a foreign, undesirable substance by the immune system of an individual treated with the polypeptide. Accordingly, it may be desirable to shield epitopes or other immunogenic determinants giving rise to an immune response in an individual treated with the polypeptide.

For this purpose it is desirable that the single-chain oligomeric polypeptide of the invention comprises one or more non-polypeptide moieties that are located so as to shield any amino acid changes as compared to the native polypeptide. The presence of such moieties may also increase the functional in vivo half-life of the polypeptide of the invention. Accordingly, in a further embodiment the single-chain oligomeric polypeptide of the invention is one wherein a non-polypeptide moiety is conjugated to an amino acid residue of any linker peptide or an amino acid residue of a monomer constituent of the single-chain oligomeric polypeptide so as to reduce the immunogenicity of the single-chain oligomeric polypeptide, and in particular any linker peptide part thereof. The conjugation may be achieved by any of the methods disclosed above.

In one embodiment, a naturally occurring TGase substrate sequence is introduced into or replaces the linker connecting the monomers in the single-chain oligomeric polypeptide, whereby the single-chain polypeptide may be modified using the highly specific TGase-catalysed cross-linking (described above).

The single-chain oligomeric polypeptide may, where desired, be conjugated to one or more non-polypeptide moieties in order to provide it with a reduced immunogenicity, a reduced clearance and/or increased functional in vivo half-life as compared to the corresponding unmodified single-chain oligomeric polypeptide.

The term “reduced immunogenicity” is intended to indicate that the conjugate gives rise to a measurably lower immune response than a reference molecule, such as wild-type TNF-α, as determined under comparable conditions. The immune response may be a cell or antibody mediated response (see, e.g., Roitt: Essential Immunology (8^{th} Edition, Blackwell) for further definition of immunogenicity). Normally, reduced antibody reactivity will be an indication of reduced immunogenicity. The reduced immunogenicity may be determined by use of any suitable method known in the art, e.g. in vivo or in vitro.

The term “functional in vivo half-life” is used in its normal meaning, i.e. the time in which 50% of a given functionality of the conjugate is retained. As an alternative to determining functional in vivo half-life, “serum half-life” may be determined, i.e. the time in which 50% of the conjugate circulates in the plasma or bloodstream prior to being cleared. Determination of serum half-life is often more simple than determining the functional in vivo half-life, and the magnitude of serum half-life is usually a good indication of the magnitude of functional in vivo half-life. Alternative terms for serum half-life include “plasma half-life”, “circulating half-life”, “serum clearance”, “plasma clearance” and “clearance half-life”. The conjugate is normally cleared by the action of one or more of the reticuloendothelial systems (RES), kidney, spleen or liver, receptor mediated elimination or by specific or unspecific proteolysis. Normally, clearance depends on size (relative to the cutoff for glomerular filtration), charge, attached carbohydrate chains, and the presence of cellular receptors for the protein. The functional in vivo half-life and the serum half-life may be determined by any suitable method known in the art, e.g. as described in the Examples section herein.

The term “increased” as used about the functional in vivo half-life or serum half-life is used to indicate that the relevant half-life of the conjugate is statistically significantly increased relative to that of a reference molecule, such as a non-conjugated TNF-α, as determined under comparable conditions.

Although it may in many cases be desirable to increase the functional in vivo half-life of the receptor antagonists of the invention, in some cases it may be desirable to maintain a relatively short in vivo half-life or serum half-life comparable to that of the ligand the antagonist is intended to block. For example, TNF-α is a cytokine that plays a central role in the immune system. Therefore, for some indications it will be desirable to obtain a short-term effect of the TNF receptor antagonist, but then to have a relatively fast clearance of the TNF receptor antagonist from the body so as to enable TNF-α to activate the TNF receptors. This is especially applicable to acute disease states or conditions caused by short-term relative increases in local or circulating TNF, for instance but not limited to post-traumatic brain damage, brain damage in relation to surgery, acute myocardial infarction, septic shock, and disseminated intravascular coagulation.

An in vivo half-life or serum half-life “comparable to” that of the ligand the antagonist is intended to block refers to the fact that the half-life of the antagonist may be specifically adapted to the half-life of the ligand. For example, the half-life of human TNF-α is a few minutes. For indications such as those mentioned above in which only a short-term antagonist effect is required, it may therefore be advantageous to have a TNF-α antagonist according to the invention with a half-life (e.g. serum half-life) of not more than a few hours or even minutes. The half-life in this case may thus be from about 1 minute to about 24 hours, such as from about 1 minute to about 1.2 hours, e.g. from about 2 minutes to about 2 hours, e.g. from about 5 minutes to about 1 hour. In some cases it may be desirable to have a half-life which is even shorter, such as not more than about 30 minutes or 15 minutes.

In general terms, the half-life of the antagonist of the invention in such cases will often be from about one tenth to about one hundred times the half-life of the ligand, typically from about one tenth to about ten times the half-life of the ligand, such as from about one half to about five times the half-life of the ligand, e.g. about one to two times the half-life of the ligand.

In such cases, the receptor antagonist of the invention preferably has a relatively low molecular weight so as to enable rapid clearance. This may be ensured by using receptor antagonists as described herein which are free of non-polypeptide moieties such as PEG and which are not in vivo glycosylated, or which only have a relatively low degree of e.g. PEGylation or glycosylation and/or which are e.g. PEGylated with PEG groups having a relatively low Mw. Given a desired clearance rate or half-life, persons skilled in the art will be able to determine how to achieve this desired effect by choosing, e.g., a suitable PEGylation or by avoiding PEGylation and/or glycosylation. Another possibility is to introduce one or more proteolytic sites into the oligomeric polypeptide so as to provide a reduction in the half-life by means of proteolysis.

Methods for Preparing Polypeptides of the Invention

The present invention further relates to a method for preparing a single-chain oligomeric polypeptide of the invention, which method comprises culturing a recombinant host cell comprising a single nucleotide sequence encoding said polypeptide in a suitable culture medium under conditions permitting expression of the nucleotide sequence and recovering the resulting polypeptide from the cell culture. In further aspects the invention relates to a nucleotide sequence encoding a single chain oligomeric polypeptide of the invention, an expression vector comprising said nucleotide sequence and a recombinant host cell comprising said sequence or said vector.

Nucleotide Sequence Encoding a Polypeptide of the Invention and its Preparation

As used herein the term “nucleotide sequence” is intended to indicate a consecutive stretch of two or more nucleotides of cDNA, genomic DNA, synthetic DNA or RNA origin. The nucleotide sequence encoding the polypeptide of the invention may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., Molecular Cloning: A Laboratory Manual, 2nd Ed., Cold Spring Harbor Laboratory, Cold Spring Harbor, N.Y., 1989). For the present purpose, the nucleotide sequence encoding the polypeptide is preferably of vertebrate origin, i.e. derived from genomic DNA or cDNA library of the relevant tissue. In particular, the nucleotide sequence may be of mammalian origin, in particular human origin.

The nucleotide sequence of the invention encoding the polypeptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

Furthermore, the nucleotide sequence may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleotide sequence, in accordance with standard techniques.

The nucleotide sequence may also be prepared by polymerase chain reaction (PCR) using specific primers, for instance as described in U.S. Pat. No. 4,683,202, U.S. Pat. No. 4,683,195 or Saiki et al., Science 239 (1988), 487-491. In general, the PCR method involves repeated cycles of primer extension synthesis, using oligonucleotide primers capable of hybridising preferentially to a template nucleic acid.

In cases where the oligomeric polypeptide comprises two or more monomeric units having an identical or highly homologous amino acid sequence, it is preferred, in order to avoid recombination between the nucleotide sequences that encode the individual monomeric units, that codon differences between these coding sequences are maximized.

In a further aspect, the invention relates to a method for producing a nucleotide sequence encoding a single-chain oligomeric polypeptide of the invention, wherein a single nucleotide sequence encoding the single-chain oligomeric polypeptide is subjected to mutagenesis so as to render at least one receptor-binding site of the encoded polypeptide-inactive and/or to increase the binding affinity of a receptor-binding site towards a ligand-binding domain of a receptor relative to a corresponding binding site of an unmodified single-chain oligomeric polypeptide or relative to the wild-type polypeptide.

Suitable mutations may be introduced by, e.g., site-directed mutagenesis as described by Sambrook et al., or by random mutagenesis or DNA shuffling, e.g. as described below followed by screening for sequences coding for polypeptides with the desired activity. Screening may be carried out by an assay method as described below.

Random mutagenesis (whether performed in the whole nucleotide sequence or one or more selected regions thereof) may be performed by any suitable method. For example, random mutagenesis is performed using a suitable physical or chemical mutagenizing agent, a suitable oligonucleotide, PCR generated mutagenesis or any combination of these mutagenizing agents/methods according to state of the art technology, e.g. as disclosed in WO 97/07202.

Error prone PCR generated mutagenesis, e.g. as described by J. O. Deshler (1992), GATA 9(4): 103-106 and Leung et al., Technique (1989) Vol. 1, No. 1, pp. 11-15, is particularly useful for mutagenesis of longer peptide stretches (corresponding to nucleotide sequences containing more than 100 bp) or entire genes, and are preferably performed under conditions that increase the misincorporation of nucleotides.

Random mutagenesis based on doped or spiked oligonucleotides is of particular use for mutagenesis of one or more regions containing shorter nucleotide sequences (normally containing less than 100 nucleotides per region). Mutagenesis of several regions is conveniently conducted by using several doped oligonucleotides and combining them by PCR. Doped or spiked oligonucleotides may also be used for random mutagenesis of nucleotide sequences encoding longer peptide stretches or entire genes when it is desirable to be able to control the random mutagenesis to a higher extent than what is possible with error prone PCR generated mutagenesis.

Conveniently, random mutagenesis of one or more selected regions of a nucleotide sequence encoding the polypeptide of interest is performed using PCR generated mutagenesis, in which one or more suitable oligonucleotide probes which flank the area to be mutagenized are used. Preferably, for mutagenesis of selected peptide stretches doped or spiked oligonucleotides are used. The doping or spiking can be designed to introduce any kind of amino acid residue and/or to avoid a codon for an unwanted amino acid residue (by lowering the amount of or completely avoiding the nucleotides resulting in this codon). The doping may be designed on the basis of the skilled person's intelligent consideration of nucleotide doping (in accordance with generally known principles), by use of a suitable algorithm, e.g. a computer program which is based on the algorithm described by Siderovski D P and Mak T W, Comput. Biol. Med. (1993) Vol. 23, No. 6, pp. 463-474 or Jensen et al. Nucleic Acids Research, 1998, Vol. 26, No. 3 or by using trinucleotides (Sondek, J. and Shortle, D., Proc. Natl. Acad. Sci, USA, Vol. 89, pp. 3581-3585, April 1992; Kayushin et al., Nucleic Acids Research, 1996, Vol. 24, No. 19, pp. 3748-3755; Virnekäs et al., Nucleic Acids Research, 1994, Vol. 22, No. 25; WO 93/21203). The doped or spiked oligonucleotide can be incorporated into the nucleotide sequence encoding the polypeptide of interest by any published technique using e.g. PCR, LCR or any DNA polymerase or ligase.

Random mutagenesis may be performed in two, three, four, five, six or more regions at the same time by synthesizing doped oligonucleotides covering each region and assembling the oligonucleotides by state of the art technologies, for example by a PCR method. One convenient PCR method involves a PCR reaction wherein the nucleotide sequence encoding the polypeptide of interest is used as a template and the doped oligonucleotides are used as primers. In addition, cloning primers localized outside the targeted regions may be used. The resulting PCR product can either be directly cloned into an appropriate expression vector or gel purified and amplified in a second PCR reaction using the cloning primers and cloned into an appropriate expression vector.

Besides substitutions the random mutagenesis may also cover random introduction of insertions or deletions. Preferably, the insertions are made so as to be in reading frame, e.g. by performing multiple introduction of three nucleotides as described by Hallet et al., Nucleic Acids Res. 1997, 25(9):1866-7 and Sondek and Shrotle, Proc Natl. Acad. Sci USA 1992, 89(8):3581-5.

The nucleotide sequence(s) or nucleotide sequence region(s) to be mutagenized are typically present on a suitable vector such as a plasmid or a bacteriophage, which as such is incubated with or otherwise exposed to the mutagenizing agent. The nucleotide sequence(s) to be mutagenized may also be present in a host cell either by being integrated into the genome of said cell or by being present on a vector harboured in the cell. Alternatively, the nucleotide sequence to be mutagenized is in isolated form. The nucleotide sequence is preferably a DNA sequence such as a cDNA, genomic DNA or synthetic DNA sequence.

In one embodiment the random mutagenesis is accompanied by conjugation to a non-polypeptide moiety. More specifically, a modified conjugated single-chain polypeptide of the invention may be prepared by

a) expressing a random mutagenized library of nucleotide sequences encoding a parent polypeptide in single-chain form,

b) conjugating one or more non-polypeptide moieties to the polypeptide variants expressed in step a),

c) screening the resulting conjugates for antagonist activity or receptor-binding, but not activating capability,

d) selecting polypeptide conjugates having such capability, and

e) optionally subjecting the nucleotide sequence encoding the polypeptide part of a polypeptide conjugate selected in step d) to one or more repeated cycles of steps a)-d).

The above method for random mutagenesis and conjugation is further described in PCT/DK00/00371.

In accordance with this embodiment the polypeptide conjugate can be prepared in a high throughput screening system allowing production and screening of a high number of different polypeptides in a short time. This is in particular suitable in the following situations:

obtaining an improved binding affinity

altering receptor specificity

creating partial antagonists

reducing/eliminating intrinsic activity of agonists

identifying optimal linkers.

Nucleotide sequence modification methods suitable for producing polypeptide variants for high throughput screening further include for instance methods which involve homologous cross-over such as disclosed in U.S. Pat. No. 5,093,257, and methods which involve gene shuffling, i.e. recombination between two or more homologous nucleotide sequences resulting in new nucleotide sequences having a number of nucleotide alterations when compared to the starting nucleotide sequences. Gene shuffling (also known as DNA shuffling) involves one or more cycles of random fragmentation and reassembly of the nucleotide sequences, followed by screening to select nucleotide sequences encoding polypeptides with desired properties. In order for homology-based nucleic acid shuffling to take place, the relevant parts of the nucleotide sequences are preferably at least 50% identical, such as at least 60% identical, more preferably at least 70% identical, such as at least 80% identical. The recombination can be performed in vitro or in vivo.

Examples of suitable in vitro gene shuffling methods are disclosed by Stemmer et al. (1994), Proc. Natl. Acad. Sci. USA; vol. 91, pp. 10747-10751; Stemmer (1994), Nature, vol. 370, pp. 389-391; Smith (1994), Nature vol. 370, pp. 324-325; Zhao et al., Nat. Biotechnol. March 1998 ; 16(3): 258-61; Zhao H. and Arnold, F B, Nucleic Acids Research, 1997, Vol. 25. No. 6 pp. 1307-1308; Shao et al., Nucleic Acids Research Jan. 15, 1998; 26(2): pp. 681-83; and WO 95/17413. An example of a suitable in vivo shuffling method is disclosed in WO 97/07205. Other techniques for mutagenesis of nucleic acid sequences by in vitro or in vivo recombination are disclosed e.g. in WO 97/20078 and U.S. Pat. No. 5,837,458. Examples of specific shuffling techniques include “family shuffling”, “synthetic shuffling” and “in silico shuffling”. Family shuffling involves subjecting a family of homologous genes from different species to one or more cycles of shuffling and subsequent screening or selection. Family shuffling techniques are disclosed e.g. by Crameri et al. (1998), Nature, vol. 391, pp. 288-291; Christians et al. (1999), Nature Biotechnology, vol. 17, pp. 259-264; Chang et al. (1999), Nature Biotechnology, vol. 17, pp. 793-797; and Ness et al. (1999), Nature Biotechnology, vol. 17, 893-896. Synthetic shuffling involves providing libraries of overlapping synthetic oligonucleotides based e.g. on a sequence alignment of homologous genes of interest. The synthetically generated oligonucleotides are recombined, and the resulting recombinant nucleic acid sequences are screened and if desired used for further shuffling cycles. Synthetic shuffling techniques are disclosed in WO 00/42561. In silico shuffling refers to a DNA shuffling procedure which is performed or modelled using a computer system, thereby partly or entirely avoiding the need for physically manipulating nucleic acids. Techniques for in silico shuffling are disclosed in WO 00/42560.

When using random mutagenesis as outlined above, the expression step a) can be conducted in any suitable manner, and conveniently as described further below. Suitably, the random mutagenized library is prepared by subjecting a nucleotide sequence encoding the parent polypeptide in single-chain form to random mutagenesis so as to create a large number of mutated nucleotide sequences. The random mutagenesis may be entirely random, both with respect to where in the nucleotide sequence the mutagenesis occurs and with respect to the nature of mutagenesis.

Alternatively, the random mutagenesis may be conducted so as to randomly mutate one or more selected regions of the polypeptide, in particular a receptor-binding site thereof. The library is typically present in a host cell, from which expression is achieved. Of particular interest is a host cell which is capable of a reasonable transformation frequency such as bacterium, e.g. E. coli, yeast, e.g. S. cereviciae, or fungus. Alternatively, a high throughput transfection system of mammalian cells or other cells capable of a desirable post-translational modification (such as in vivo glycosylation) may be employed and examples include CHO (Chinese Hamster Ovary) and COS and BHK (Baby Hamster Kidney) cells.

Conjugation step b) is conveniently conducted as described above in connection with conjugation to a polymer or an oligosaccharide moiety. The screening step c) is an important element of the method according to this embodiment of the invention. The screening is conveniently conducted as a primary screening for receptor-binding, but not activating capability, e.g. based on the principles disclosed in the section entitled “Assay”.

In a preferred embodiment as many as possible of steps a-d) are performed in a high throughput screening system. In particular, it is preferred that steps a)-d) are performed in a robotized system, wherein the expression from the random mutagenized library of nucleotide sequences is achieved in microtiter plates, the resulting supernatant is transferred to a different microtiter plate, preferably under conditions allowing immobilization of the polypeptides, and optionally under conditions where the receptor-binding site of the polypeptide is blocked, e.g. by a suitable receptor, receptor analogue or antibody, and/or the polypeptide is provided with a tag, e.g. a His tag known in the art (such as His-His-His-His-His-His; Met-Lys-His-His-His-His-His-His; Met-Lys-His-His-Ala-His-His-Gln-His-His; or Met-Lys-His-Gln-His-Gln-His-Gln-His-Gln-His-Gln-His-Gln (all available from Unizyme Laboratories, Denmark); the optionally immobilized, blocked and/or tagged polypeptides are subjected to conjugation to a non-polypeptide moiety while present in the microtiter plate and the resulting polypeptide conjugates present in the microtiter plate are subjected to the relevant screening. Subsequently, selected positive polypeptide conjugates are subjected to further characterization including secondary screening.

It should of course be understood that not all vectors and expression control sequences function equally well to express the nucleotide sequence encoding the polypeptide described herein. Neither will all hosts function equally well with the same expression system. However, one of skill in the art will be able to make a selection among these vectors, expression control sequences and hosts without undue experimentation. For example, in selecting a vector, the host must be considered because the vector must replicate in it or be able to integrate into the chromosome. The vector's copy number, the ability to control that copy number, and the expression of any other proteins encoded by the vector, such as antibiotic markers, should also be considered. In selecting an expression control sequence, a variety of factors should also be considered. These include, for example, the relative strength of the sequence, its controllability, and its compatibility with the nucleotide sequence encoding the polypeptide, particularly as regards potential secondary structures. Hosts should be selected by consideration of their compatibility with the chosen vector, the toxicity of the product coded for by the nucleotide sequence, their secretion characteristics, their ability to fold the polypeptide correctly, their fermentation or culture requirements, and the ease of purification of the products coded for by the nucleotide sequence.

The recombinant vector may be an autonomously replicating vector, i.e. a vector which exists as an extrachromosomal entity, the replication of which is independent of chromosomal replication, e.g. a plasmid. Alternatively, the vector is one which, when introduced into a host cell, is integrated into the host cell genome and replicated together with the chromosome(s) into which it has been integrated.

The vector is preferably an expression vector in which the nucleotide sequence encoding the polypeptide of the invention is operably linked to additional segments required for transcription of the nucleotide sequence. The vector is typically derived from plasmid or viral DNA. A number of suitable expression vectors for expression in the host cells mentioned herein are commercially available or described in the literature. Useful expression vectors for eukaryotic hosts, include, for example, vectors comprising expression control sequences from SV40, bovine papilloma virus, adenovirus and cytomegalovirus. Specific vectors are, e.g., pCDNA3.1(+)\Hyg (Invitrogen, Carlsbad, Calif., USA) and pCI-neo (Stratagene, La Jolla, Calif., USA). Useful expression vectors for bacterial hosts include known bacterial plasmids, such as plasmids from E. coli, including pBR322, pET3a and pET12a (both from Novagen Inc., Wis., USA), wider host range plasmids, such as RP4, phage DNAs, e.g., the numerous derivatives of phage lambda, e.g., NM989, and other DNA phages, such as M13 and filamentous single stranded DNA phages. Useful expression vectors for yeast cells include the 2μ plasmid and derivatives thereof, the POT1 vector (U.S. Pat. No. 4,931,373), the pJSO37 vector described in (Okkels, Ann. New York Acad. Sci. 782, 202-207, 1996) and pPICZ A, B or C (Invitrogen). Useful vectors for insect cells include pBluebac 4.5 and pMelbac (both available from Invitrogen).

Other vectors for use in this invention include those that allow the nucleotide sequence encoding the polypeptide to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, “Construction Of A Modular Dihydrafolate Reductase cDNA Gene: Analysis Of Signals Utilized For Efficient Expression”, Mol. Cell. Biol., 2, pp. 1304-19 (1982)) and glutamine synthetase (“GS”) amplification (see, e.g., U.S. Pat. No. 5,122,464 and EP 338,841).

The recombinant vector may further comprise a DNA sequence enabling the vector to replicate in the host cell in question. An example of such a sequence (when the host cell is a mammalian cell) is the SV40 origin of replication. When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pyrG, arcB, niaD, sC.

The term “control sequences” is defined herein to include all components which are necessary or advantageous for the expression of the polypeptide of the invention. Each control sequence may be native or foreign to the nucleic acid sequence encoding the polypeptide. Such control sequences include, but are not limited to, a leader, polyadenylation sequence, propeptide sequence, promoter, enhancer or upstream activating sequence, signal peptide sequence, and transcription terminator. At a minimum, the control sequences include a promoter.

A wide variety of expression control sequences may be used in the present invention. Such useful expression control sequences include the expression control sequences associated with structural genes of the foregoing expression vectors as well as any sequence known to control the expression of genes of prokaryotic or eukaryotic cells or their viruses, and various combinations thereof.

Examples of suitable control sequences for directing transcription in mammalian cells include the early and late promoters of SV40 and adenovirus, e.g. the adenovirus 2 major late promoter, the MT-1 (metallothionein gene) promoter, the human cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus Elb region polyadenylation signals and the Kozak consensus sequence (Kozak, M. J Mol Biol Aug. 20, 1987; 196(4):947-50).

In order to improve expression in mammalian cells a synthetic intron may be inserted in the 5′ untranslated region of the nucleotide sequence encoding the polypeptide of interest. An example of a synthetic intron is the synthetic intron from the plasmid pCI-Neo (available from Promega Corporation, Wis., USA).

Examples of suitable control sequences for directing transcription in insect cells include the polyhedrin promoter, the P10 promoter, the Autographa californica polyhedrosis virus basic protein promoter, the baculovirus immediate early gene 1 promoter and the baculovirus 39K delayed-early gene promoter, and the SV40 polyadenylation sequence.

Examples of suitable control sequences for use in yeast host cells include the promoters of the yeast α-mating system, the yeast triose phosphate isomerase (TPI) promoter, promoters from yeast glycolytic genes or alcohol dehydogenase genes, the ADH2-4c promoter and the inducible GAL promoter.

Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulans glucoamylase, A. nidulans acetamidase, Rhizomucor miehei aspartic proteinase or lipase, the TPI1 terminator and the ADH3 terminator.

Examples of suitable control sequences for use in bacterial host cells include promoters of the lac system, the trp system, the TAC or TRC system and the major promoter regions of phage lambda.

To direct a polypeptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the polypeptide in the correct reading frame. Secretory signal sequences are commonly positioned 5′ to the-DNA sequence encoding the polypeptide. The secretory signal sequence may be that normally associated with the polypeptide or may be from a gene encoding another secreted protein.

For secretion from yeast cells, the secretory signal sequence may encode any signal peptide, which ensures efficient direction of the expressed polypeptide into the secretory pathway of the cell. The signal peptide may be naturally occurring signal peptide, or a functional part thereof, or it may be a synthetic peptide. Suitable signal peptides have been found to be the a-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR 1 signal peptide (cf. WO 87/02670), and the yeast aspartic protease 3 (YAP3) signal peptide (cf. M. Egel-Mitani et al., Yeast 6, 1990, pp. 127-137).

In addition, to obtain an efficient secretion, a pro-peptide encoding sequence may be inserted downstream of the signal sequence and upstream of the nucleotide sequence encoding the polypeptide. A pro-peptide may be the yeast α-factor pro-peptide (the use of which is described in e.g. U.S. Pat. No. 4,546,082, EP 16 201, EP 123 294, EP 123 544 and EP 163 529) or a synthetic pro-peptide (WO 89/02463, WO 92/11378 or WO98/32867).

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase.

For use in insect cells, the signal peptide may conveniently be derived from an insect gene (cf. WO 90/05783), such as the lepidopteran Manduca sexta adipokinetic hormone precursor, (cf. U.S. Pat. No. 5,023,328), the honeybee melittin (Invitrogen), ecdysteroid UDPglucosyltransferase (egt) (Murphy et al., Protein Expression and Purification 4, 349-357 (1993) or human pancreatic lipase (hpl) (Methods in Enzymology 284, pp. 262-272, 1997).

For use in mammalian cells, a suitable signal sequence is the murine Ig kappa light chain signal sequence (Coloma, M (1992) J. Imm. Methods 152:89-104) or the signal sequence naturally associated with the nucleotide sequence encoding the polypeptide.

The procedures used to ligate the DNA sequences coding for-the polypeptide of the invention, the promoter and optionally the terminator and/or secretory signal sequence, respectively, and to insert them into suitable vectors containing the information necessary for replication, are well known to persons skilled in the art (cf., for instance, Sambrook et al., op.cit.).

Any suitable host may be used to produce the polypeptide, including bacteria, fungi (including yeasts) and higher eukaryotic cells (including plant, insect and mammalian cells, and other appropriate animal cells and cell lines), as well as transgenic animals or plants.

Examples of bacterial host cells include gram-positive bacteria such as strains of Bacillus, e.g. B. brevis or B. subtilis, Pseudomonas or Streptomyces, or gram-negative bacteria, such as strains of E. coli. The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

Examples of suitable filamentous fungal host cells include strains of Aspergillus, e.g. A. oryzae, A. niger, or A. nidulans, Fusarium or Trichodenna. Fungal cells may be transformed by a process involving protoplast formation, transformation of the protoplasts, and regeneration of the cell wall in a manner known per se. Suitable procedures for transformation of Aspergillus host cells are described in EP 238 023 and U.S. Pat. No. 5,679,543. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

Examples of suitable yeast host cells include strains of Saccharomyces, e.g. S. cerevisiae, Schizosaccharomyces, Klyveromnyces, Pichia, such as P. pastoris or P. methanolica, Hansenula, such as H. polymorpha or Yarrowia. Methods for transforming yeast cells with heterologous DNA and producing heterologous polypeptides therefrom are disclosed by Clontech Laboratories, Inc, Palo Alto, Calif., USA (in the product protocol for the Yeastmaker™ Yeast Tranformation System Kit), and by Reeves et al., FEMS Microbiology Letters 99 (1992) 193-198, Manivasakam and Schiestl, Nucleic Acids Research, 1993, Vol. 21, No. 18, pp. 4414-4415 and Ganeva et al., FEMS Microbiology Letters 121 (1994) 159-164.

Examples of suitable insect host cells include a Lepidoptora cell line, such as Spodoptera frugiperda (Sf9 or Sf21) or Trichoplusioa ni cells (High Five) (U.S. Pat. No. 5,077,214). Transformation of insect cells and production of heterologous polypeptides therein may be performed as described by Invitrogen.

Examples of suitable mammalian host cells include Chinese hamster ovary (CHO) cell lines, (e.g. CHO-K1; ATCC CCL-61), Green Monkey cell lines (COS) (e.g. COS 1 (ATCC CRL-1650), COS 7 (ATCC CRL-1651)); mouse cells (e.g. NS/O), Baby Hamster Kidney (BHK) cell lines (e.g. ATCC CRL-1632 or ATCC CCL-10), and human cells (e.g. HEK 293 (ATCC CRL-1573)), as well as plant cells in tissue culture. Additional suitable cell lines are known in the art and available from public depositories such as the American Type Culture Collection, Rockville, Md.

Methods for introducing exogeneous DNA into mammalian host cells include calcium phosphate-mediated transfection, electroporation, DEAE-dextran mediated transfection, liposome-mediated transfection, viral vectors and the transfection method described by Life Technologies Ltd, Paisley, UK using Lipofectamin 2000. These methods are well-known in the art and e.g. described by Ausbel et al. (eds.), 1996, Current Protocols in Molecular Biology, John Wiley & Sons, New York, USA. The cultivation of mammalian cells are conducted according to established methods, e.g. as disclosed in (Animal Cell Biotechnology, Methods and Protocols, Edited by Nigel Jenkins, 1999, Human Press Inc, Totowa, N.J., USA and Harrison M A and Rae I F, General Techniques of Cell Culture, Cambridge University Press 1997).

In order to produce a glycosylated polypeptide a eukaryotic host cell, e.g. of the type mentioned above, is preferably used.

In the production method of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermenters performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from the periplasmic space or from various cell lysates. The polypeptide produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, hydrophobic interaction chromatography, immobilised metal ion affinity chromatography, or the like, dependent on the type of polypeptide in question.

Assay

In order to determine whether a single-chain oligomeric polypeptide of the invention can bind to a receptor, e.g. a TNF receptor such as TNF-R1 or TNF-R2, and whether it can compete with natural ligands of the receptor(s), ligand receptor binding assays are performed.

The strength of the binding between a receptor and ligand can be measured using an enzyme-linked immunoadsorption assay (ELISA), a radio immunoassay (RIA), a binding assay, for instance based on the scintillation proximity assay (SPA) technique or other such detection methods which are techniques well known in the art. The ligand-receptor binding interaction may also be measured with the BIACORE® instrument, which exploits plasmon resonance detection. Protein samples are flowed over chips containing a covalently attached capturing protein (typically receptors or antibodies), and interactions between the chip and the sample are measured by an increase in the surface plasmon resonance of the chip. The amount of bound protein can thereby be compared to a known standard and the concentration can be determined.

In another assay, transfected mammalian cells expressing a receptor of interest and a luciferase (or other) reporter gene construct driven by a suitable promoter are exposed to a submaximal concentration of wild-type polypeptide and one or more muteins to be tested for antagonist activity. The activity of luciferase may be measured in a standard luciferase assay (e.g. a LucLite™ kit, produced by Packard). The degree of antagonism may be estimated from the ability of the muteins to block luciferase expression. Dose-response curves may be prepared for all potent antagonists.

With any of these or other techniques for measuring receptor-ligand interactions, one can evaluate the quality of a single-chain oligomeric polypeptide of the invention. Once it is established that a single-chain oligomeric polypeptide of the invention can bind to a given receptor and preferably, that it can compete with endogenous ligands for receptor binding, an assay to monitor the activity of the receptor once it is bound to the polypeptide must be performed. Assays known to be reliable indicators of signalling by the selected receptor will, of course, be preferred. In any event, at least one activity associated with the activation of the selected receptor should be measured to assess the ability of the potential TNF-α heteromeric inhibitor, when bound to the selected receptor, to inhibit receptor signalling. Specific assays for the above purposes are described in the Example section herein.

As indicated above, the invention comprises a method for identifying a single-chain oligomeric polypeptide as described herein, the method comprising contacting one or more test polypeptides with (a cell expressing) an appropriate cellular receptor for: said oligomeric polypeptide and identifying polypeptides that bind to and inhibit activation of said receptor.

In a preferred embodiment, each test polypeptide may form part of a series of test polypeptides, wherein each test polypeptide carries one or more modifications within a receptor-binding site or presumed receptor-binding site thereof such that at least one of the modifications present in each test polypeptide is different from the modifications present in the remainder of the test polypeptides. The series of test polypeptides may for instance be prepared by random mutagenesis of a nucleotide sequence encoding the wild-type polypeptide.

Pharmaceutical Use and Formulations

As indicated above, the oligomeric polypeptide of the invention is contemplated to be advantageous for use in therapeutic applications where it is desirable to inhibit activation of receptors that require binding of an oligomeric ligand to two or more receptor subunits to be activated.

Pharmaceutical formulations of the polypeptide or conjugate of the invention are typically administered in a composition that includes one or more pharmaceutically acceptable carriers or excipients. Such pharmaceutical compositions may be prepared in a manner known per se in the art to result in a polypeptide pharmaceutical that is sufficiently storage-stable and is suitable for administration to humans or animals.

Drug Form

The polypeptide or conjugate of the invention can be used “as is” and/or in a salt form thereof. Suitable salts include, but are not limited to, salts with alkali metals or alkaline earth metals, such as sodium, potassium, calcium and magnesium, as well as e.g. zinc salts. These salts or complexes may by present as a crystalline and/or amorphous structure.

Excipients

“Pharmaceutically acceptable” means a carrier or excipient that at the dosages and concentrations employed does not cause any untoward effects in the patients to whom it is administered. Such pharmaceutically acceptable carriers and excipients are well known in the art (see Remington's Pharmaceutical Sciences, 18th edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]).

Dose

The polypeptides and conjugates of the invention will be administered to patients in a therapeutically effective dose. By “therapeutically effective dose” herein is meant a dose that is sufficient to produced the desired effects in relation to the condition for which it is administered. The exact dose will depend on the disorder to be treated, and will be ascertainable by one skilled in the art using known techniques.

Mix of Drugs

The pharmaceutical composition of the invention may be administered alone or in conjunction with other therapeutic agents. These agents may be incorporated as part of the same pharmaceutical composition or may be administered separately from the polypeptide or conjugate of the invention, either concurrently or in accordance with another treatment schedule. In addition, the polypeptide, conjugate or pharmaceutical composition of the invention may be used as an adjuvant to other therapies.

Patients

A “patient” for the purposes of the present invention includes both humans and other mammals. Thus the methods are applicable to both human therapy and veterinary applications.

Types of Composition and Administration Route

The pharmaceutical composition of the polypeptide or conjugate of the invention may be formulated in a variety of forms, e.g. as a liquid, gel, lyophilized, or as a compressed solid. The preferred form will depend upon the particular indication being treated and will be readily able to be determined by one skilled in the art.

The administration of the formulations of the present invention can be performed in a variety of ways, including, but not limited to, orally, subcutaneously, intravenously, intracerebrally, intranasally, transdermally, intraperitoneally, intramuscularly, intrapulmonary, vaginally, rectally, intraocularly, or in any other acceptable manner. The formulations can be administered continuously by infusion, although bolus injection is acceptable, using techniques well known in the art, such as pumps or implantation. In some instances the formulations may be directly applied as a solution or spray.

Parenterals

An example of a pharmaceutical composition is a solution designed for parenteral administration. Although in many cases pharmaceutical solution formulations are provided in liquid form, appropriate for immediate use, such parenteral formulations may also be provided in frozen or in lyophilized form. In the former case, the composition must be thawed prior to use. The latter form is often used to enhance the stability of the active compound contained in the composition under a wider variety of storage conditions, as it is recognized by those skilled in the art that lyophilized preparations are generally more stable than their liquid counterparts. Such lyophilized preparations are reconstituted prior to use by the addition of one or more suitable pharmaceutically acceptable diluents such as sterile water for injection or sterile physiological saline solution.

In case of parenterals, they are prepared for storage as lyophilized formulations or aqueous solutions by mixing, as appropriate, the polypeptide having the desired degree of purity with one or more pharmaceutically acceptable carriers, excipients or stabilizers typically employed in the art (all of which are termed “excipients”), for example buffering agents, stabilizing agents, preservatives, isotonifiers, non-ionic detergents, antioxidants and/or other miscellaneous additives.

Buffering agents help to maintain the pH in the range which approximates physiological conditions. They are typically present at a concentration ranging from about 2 mM to about 50 mM Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities are phosphate buffers, histidine buffers and trimethylamine salts such as Tris.

Preservatives are added to retard microbial growth, and are typically added in amounts of e.g. about 0.1%-2% (w/v). Suitable preservatives for use with the present invention include phenol, benzyl alcohol, meta-cresol, methyl paraben, propyl paraben, octadecyldimethylbenzyl ammonium chloride, benzalkonium halides (e.g. benzalkonium chloride, bromide or iodide), hexamethonium chloride, alkyl parabens such as methyl or propyl paraben, catechol, resorcinol, cyclohexanol and 3-pentanol.

Isotonicifiers are added to ensure isotonicity of liquid compositions and include polyhydric sugar alcohols, preferably trihydric or higher sugar alcohols, such as glycerin, erythritol, arabitol, xylitol, sorbitol and mannitol. Polyhydric alcohols can be present in an amount between 0.1% and 25% by weight, typically 1% to 5%, taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, omithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thiosulfate; low molecular weight polypeptides (i.e. <10 residues); proteins such as human serum albumin, bovine-serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on the active protein weight.

Non-ionic surfactants or detergents (also known as “wetting agents”) may be present to help solubilize the therapeutic agent as well as to protect the therapeutic polypeptide against agitation-induced aggregation, which also permits the formulation to be exposed to shear surface stress without causing denaturation of the polypeptide. Suitable non-ionic surfactants include polysorbates (20, 80, etc.), polyoxamers (184, 188 etc.), Pluronic® polyols, polyoxyethylene sorbitan monoethers (Tween®-20, Tween®-80, etc.).

Additional miscellaneous excipients include bulking agents or fillers (e.g. starch), chelating agents (e.g. EDTA), antioxidants (e.g., ascorbic acid, methionine, vitamin E) and cosolvents.

The active ingredient may also be entrapped in microcapsules prepared, for example, by coascervation techniques or by interfacial polymerization, for example hydroxymethylcellulose, gelatin or poly-(methylmethacylate) microcapsules, in colloidal drug delivery systems (for example liposomes, albumin microspheres, microemulsions, nano-particles and nanocapsules) or in macroemulsions. Such techniques are disclosed in Remington's Pharmaceutical Sciences, supra.

Parenteral formulations to be used for in vivo administration must be sterile. This is readily accomplished, for example, by filtration through sterile filtration membranes.

Sustained Release Preparations

Suitable examples of sustained-release preparations include semi-permeable matrices of solid hydrophobic polymers containing the polypeptide or conjugate, the matrices having a suitable form such as a film or microcapsules. Examples of sustained-release matrices include polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, degradable lactic acid-glycolic acid copolymers such as the ProLease® technology or Lupron Depot® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for long periods such as up to or over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S bond formation through thio-disulfide interchange, stabilization may be achieved by modifying sulfhydryl residues, lyophilizing from acidic solutions, controlling moisture content, using appropriate additives, and developing specific polymer matrix compositions.

Oral Administration

For oral administration, the pharmaceutical composition may be in solid or liquid form, e.g. in the form of a capsule, tablet, suspension, emulsion or solution. The pharmaceutical composition is preferably made in the form of a dosage unit containing a given amount of the active ingredient. A suitable daily dose for a human or other mammal may vary widely depending on the condition of the patient and other factors, but can be determined by persons skilled in the art using routine methods.

Solid dosage forms for oral administration may include capsules, tablets, suppositories, powders and granules. In such solid dosage forms, the active compound may be admixed with at least one inert diluent such as sucrose, lactose, or starch. Such dosage forms may also comprise, as is normal practice, additional substances, e.g. lubricating agents such as magnesium stearate. In the case of capsules, tablets and pills, the dosage forms may also comprise buffering agents. Tablets and pills can additionally be prepared with enteric coatings.

The polypeptides or conjugates may be admixed with adjuvants such as lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinyl-pyrrolidine, and/or polyvinyl alcohol, and tableted or encapsulated for conventional administration. Alternatively, they may be dissolved in saline, water, polyethylene glycol, propylene glycol, ethanol, oils (such as corn oil, peanut oil, cottonseed oil or sesame oil), tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be subjected to conventional pharmaceutical operations such as sterilization and/or may contain conventional adjuvants such as preservatives, stabilizers, wetting agents, emulsifiers, buffers, fillers, etc., e.g. as disclosed elsewhere herein.

Liquid dosage forms for oral administration may include pharmaceutically acceptable emulsions, solutions, suspensions, syrups and elixirs containing inert diluents commonly used in the art, such as water. Such compositions may also comprise adjuvants such as wetting agents, sweeteners, flavoring agents and perfuming agents.

Suppositories

Suppositories for rectal administration of the polypeptide or conjugate can be prepared by mixing the active compound with a suitable non-irritating excipient such as cocoa butter and polyethylene glycols that are solid at ordinary temperatures but liquid at the rectal temperature and will therefore melt in the rectum and release the drug.

Topical Administration

Formulations suitable for topical administration include liquid or semi-liquid preparations suitable for penetration through the skin (e.g., liniments, lotions, ointments, creams, or pastes) and drops suitable for administration to the eye, ear, or nose.

Pulmonary Delivery

Formulations suitable for use with a nebulizer, either jet or ultrasonic, will typically comprise the polypeptide or conjugate dissolved in water at a concentration of, e.g., about 0.01 to 25 mg of conjugate per mL of solution, preferably about 0.1 to 10 mg/mL. The formulation may also include a buffer and a simple sugar (e.g., for protein stabilization and regulation of osmotic pressure), and/or human serum albumin ranging in concentration from 0.1 to 10 mg/ml. Examples of buffers that may be used are sodium acetate, citrate and glycine. Preferably, the buffer will have a composition and molarity suitable to adjust the solution to a pH in the range of 3 to 9. Generally, buffer molarities of from 1 mM to 50 mM are suitable for this purpose. Examples of sugars which can be utilized are lactose, maltose, mannitol, sorbitol, trehalose, and xylose, usually in amounts ranging from 1% to 10% by weight of the formulation.

The nebulizer formulation may also contain a surfactant to reduce or prevent surface induced aggregation of the protein caused by atomization of the solution in forming the aerosol. Various conventional surfactants can be employed, such as polyoxyethylene fatty acid esters and alcohols, and polyoxyethylene sorbitan fatty acid esters. Amounts will generally range between 0.001% and 4% by weight of the formulation. An especially preferred surfactant for purposes of this invention is polyoxyethylene sorbitan monooleate.

Specific formulations and methods of generating suitable dispersions of liquid particles of the invention are described in WO 94/20069, U.S. Pat. No. 5,915,378, U.S. Pat. No. 5,960,792, U.S. Pat. No. 5,957,124, U.S. Pat. No. 5,934,272, U.S. Pat. No. 5,915,378, U.S. Pat. No. 5,855,564, U.S. Pat. No. 5,826,570 and U.S. Pat. No. 5,522,385, which are hereby incorporated by reference.

Formulations for use with a metered dose inhaler device will generally comprise a finely divided powder. This powder may be produced by lyophilizing and then milling a liquid conjugate formulation and may also contain a stabilizer such as human serum albumin (HSA). Typically, more than 0.5% (w/w) HSA is added. Additionally, one or more sugars or sugar alcohols may be added to the preparation if necessary. Examples include lactose maltose, mannitol, sorbitol, sorbitose, trehalose, xylitol, and xylose. The amount added to the formulation can range from about 0.01 to 200% (w/w), preferably from approximately 1 to 50%, of the conjugate present. Such formulations are then lyophilized and milled to the desired particle size.

The properly sized particles are then suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant. This mixture is then loaded into the delivery device.

Formulations for powder inhalers will comprise a finely divided dry powder containing conjugate and may also include a bulking agent, such as lactose, sorbitol, sucrose, or mannitol in amounts which facilitate dispersal of the powder from the device, e.g., 50% to 90% by weight of the formulation. The particles of the powder shall have aerodynamic properties in the lung corresponding to particles with a density of about 1 g/cm² having a median diameter less than 10 micrometers, preferably between 0.5 and 5 micrometers, most preferably of between 1.5 and 3.5 micrometers.

The powders for these devices may be generated and/or delivered by methods disclosed in U.S. Pat. No. 5,997,848, U.S. Pat. No. 5,993,783, U.S. Pat. No. 5,985,248, U.S. Pat. No. 5,976,574, U.S. Pat. No. 5,922,354, U.S. Pat. No. 5,785,049 and U.S. Pat. No. 5,654,007.

Mechanical devices designed for pulmonary delivery of therapeutic products, include but are not limited to nebulizers, metered dose inhalers, and powder inhalers, all of which are familiar to those of skill in the art. Specific examples of commercially available devices suitable for the practice of this invention are the Ultravent™ nebulizer, manufactured by Mallinckrodt, Inc., St. Louis, Mo.; the Acorn™ II nebulizer, manufactured by Marquest Medical Products, Englewood, Colo.; the Ventolin™ metered dose inhaler, manufactured by Glaxo Inc., Research Triangle Park, N.C.; the Spinhaler™ powder inhaler, manufactured by Fisons Corp., Bedford, Mass.; the “standing cloud” device of Inhale Therapeutic Systems, Inc., San Carlos, Calif.; the AIR™ inhaler manufactured by Alkermes, Cambridge, Mass.; and the AERxr™ pulmonary drug delivery system manufactured by Aradigm Corporation, Hayward, Calif.

The general principles of the invention will in the following be illustrated using TNF-α and VEGF as examples of a trimer and a dimer, respectively. It will be understood by persons skilled in the art that these general principles illustrated using these polypeptides will be applicable in a similar manner to other oligomeric single-chain polypeptide antagonists in accordance with the teachings found elsewhere herein.

TNF-α

TNF-α: Background

Tumour Necrosis Factor (TNF)-related cytokines have emerged as a large family of mediators of host defense and immune regulation. Members of this family exist in membrane-bound forms, which act locally through cell-cell contact, or as secreted proteins that can act on local as well as distant targets. A parallel family of TNF-related receptors react with these molecules and trigger a variety of pathways including cell death, cell proliferation, tissue differentiation and pro-inflammatory responses.

The TNF family comprises at least 17 ligands (including TNF-α, LT-α, LT-β, FasL, CD40L, CD30L, CD27L, Ox40L, 4-lBBL, RANKL, TRAIL, TWEAK, LIGHT, TRANCE, APRIL, THANK and TALL-1), and an even larger number of receptors ((TNF-R1, TNF-R2, LT-β-R, Fas, CD40R, CD30, CD27, Ox40, 4-1BB, RANK, TR1-4, TRAMP, and HVEM) (Smith et al., Cell, 76, pp. 959-62 (1994)).

Tumour necrosis factor α (TNF-α; or cachectin) is a pleiotropic cytokine that plays a central role in the control of the immune system. TNF-α is primarily secreted by macrophages but also by a number of other cells including lymphocytes, mast cells, neutrophils, smooth muscle cells and tumour cells.

Human TNF-α monomer is expressed as a membrane-bound 26 kDa non-glycosylated precursor molecule that is subsequently cleaved by a metalloprotease enzyme (TNF-α converting enzyme, TACE) to a 17 kDa soluble form. Both the membrane bound and the soluble form of TNF-α form homo-trimers, which are the active forms of the molecule.

The mature soluble human TNF-α monomeric molecule consists of 157 amino acids, the sequence of which is disclosed e.g. by Pennica et al. (Nature 312: 724-729, 1984). As used herein, amino acid residue position numbers of TNF-α refer to the 157 amino acid sequence of the wild-type human polypeptide. It should be noted in this regard that for single-chain polypeptides comprising three TNF-α monomers, each monomeric unit will for the sake of simplicity be numbered separately starting at position one.

The atomic structure of TNF-α has been solved by X-ray crystallography by Eck & Sprang 1989, J. Biol. Chem. 264: 17595-17605 and by Jones, Stuart & Walker 1989, Nature 338: 225-228. The atomic coordinates of the structure determined by Eck & Sprang are available via the Protein Data Bank (PDB) (Bernstein et al. J. Mol. Biol. (1977) 112 pp. 535) and electronically available via The Research Collaboratory for Structural Bioinformatics PDB at http://www.rcsb.orgipdb/ under accession code 1TNF.

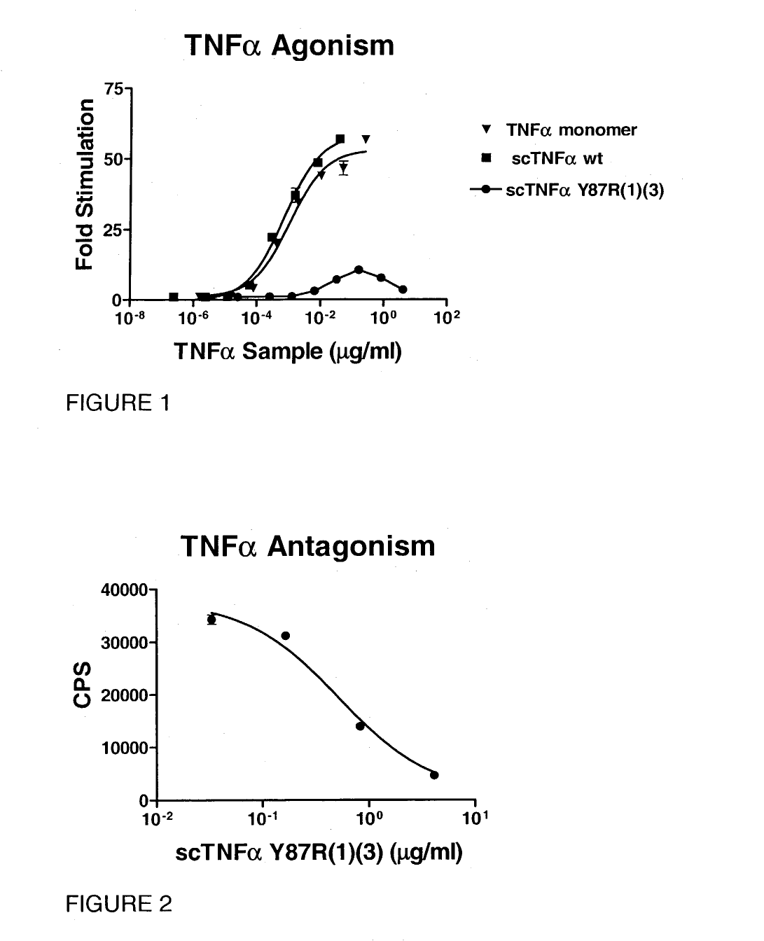

The structure by Eck & Sprang was determined at 2.6 Å resolution. Three monomers in close contact about a 3-fold axis of symmetry form a compact bell-shaped trimer. Each monomer forms an elongated, antiparallel β-pleated sandwich with a “jelly-roll” topology consisting almost entirely of antiparallel β-strands. The first 5 amino-terminal residues could not be located for any monomer in the electron density map and are presumed to be disordered. It is interesting to note that the N-terminal is close in space to both the C-terminal of the same monomer and the C-terminal of the next monomer in the naming sequence (i.e. molecule 1 C-terminal close to molecule 2 N-terminal, molecule 2 C-terminal close to molecule 3 N-terminal and molecule 3 N-terminal close to molecule 1 C-terminal (see FIG. 1). More specifically, the amino acid residue located in position 9 from the N-terminal is closest to the C-terminal of the “following” chain, the distances between the closest oxygen atom of the C-terminal residue and the N-atom of residue 9 are 7.42 Å (1-2), 5.43 Å (2-3) and 6.29 Å (3-1). A close structural homology between TNF-α and the coat proteins of icosahedral RNA viruses as well as with the influenza hemagglutinin has been noted.

Another structure of mature TNF-α by Jones, Stuart & Walker was determined at 2.9 Å resolution. Here the first 4 amino-terminal residues could not be located for any monomer in the electron density map and are presumed to be disordered. The description of the structure is essentially the same as for the structure determined by Eck & Sprang.

TNF-α elicits its effects through two different receptors, referred to as TNF-R1 and TNF-R2 (also known as p55 or p60 receptor and p75 or p80 receptor, respectively) (Naismith & Sprang, J. Inflamm. 1995-96;47(1-2):1-7). The TNF receptors belong to the group of death domain (DD) receptors. They consist of a large extracellular domain, a single membrane spanning α-helix and an intracellular signalling domain. Binding of trimeric TNF-α or human lymphotoxin (LT-α, also known as TNF-β) to the extracellular segment of the receptor induces activation of homotrimeric receptors, each consisting of three TNF receptor molecules. The activated receptors recruit a number of intracellular proteins (e.g. TRADD, FADD, and TRAF) that elicit different signalling cascades-for instance activation of the transcription factor NF-κB, activation of the MAP kinases, and caspases. The extracellular parts of the two receptor subtypes are 28% identical while the cytoplasmatic domains share no apparent homology.

TNF-R1 seems to mediate the majority of the TNF-α effects, while the role of TNF-R2 is less clear. However, studies using mice with targeted disruption of TNF-R1 and/or -R2 indicate that TNF-R2 has a permissive function on the TNF-α signal mediated by TNF-R1 (Peschon, et al., J. Immunol. Jan. 15, 1998 ;160(2):943-52).

X-ray crystallographic studies on the extracellular domain of the TNF-R1 receptor have been reported by Naismith et al. 1996, Structure 4:1251-1262. Furthermore, the X-ray crystal structure of LT-α in complex with the extracellular domain of the TNF-R1 receptor has been determined by Banner et al. 1993 Cell 73:431-445. The structure was determined at 2.85 Å resolution and shows a complex with three receptor molecules arranged symmetrically around the LT-α trimer. The structure reveals the receptor-binding site as being at the interface between two LT-α monomers and the structure defines the orientation of the LT-α ligand with respect to the cell membrane concluding that the N- and C-terminal of LT-α are placed at the end of the molecule pointing away from the membrane.

TNF-R activation is associated with immune and inflammatory reactions, septic shock, autoimmune disorders and graft-host disease (Beutler, B., Tumour Necrosis Factors: The Molecules and Their Emerging Role in Medicine. New York: Raven Press (1994)). It would be useful in many instances to treat disorders associated with TNF-R by selectively inhibiting TNF-R signalling.

TNF-α is central in mediation of responses to tissue injury, protection of the host from microbial infections, and destruction of tumours. It is primarily pro-inflammatory and acts both directly on the target cells and indirectly through stimulation of production of other cytokines. It has thus been shown that TNF-α is a very important factor in the regulation of secretion of both pro-inflammatory cytokines such as interleukins 1, 6, and 8, and granulocyte-macrophage colony stimulating factor (GM-CSF) and anti-inflammatory cytokines (e.g. interleukin 10, interleukin 1 receptor antagonist, and transforming growth factor b (TGF-β)). Furthermore, TNF-α exerts a variety of actions including: stimulation of lipoprotein lipase synthesis, activation of polymorphonuclear leukocytes, regulation of cell growth, cytotoxic action on certain cell types, antiviral activity, stimulation of bone resorption, and a number of immunoregulatory actions, including activation of T and B cells, monocytes, thymocytes, and induction of major histocompatibility complex expression.

Excessive production of TNF-α has been implicated in the pathogenicity of a wide range of diseases such as rheumatoid arthritis, Crohn's disease, systemic lupus erythematosus, Sjögren's disease, cachexia, septic shock, diabetes mellitus, myasthenia gravis, post-traumatic brain damage, myocardial infarction, post-traumatic brain-damage, and other destructive processes related to stress or activation of the inflammatory system. This has been substantiated by the finding that blockade of the actions of TNF-α is a potent treatment of several of these disease states in both animal models and patients (Maini, Elliott, et al. APMIS April 1997;105(4):257-63).

Currently, two different anti-TNF-α strategies are employed in the treatment of Crohn's disease and rheumatoid arthritis: 1) Infliximab, a humanized monoclonal antibody targeted against TNF-α, 2) Ethanercept, a TNF-α receptor-Fc fusion protein. Both of these strategies are indirect since they block the actions of TNF-α by eliminating the cytokine rather than preventing TNF-α from activating its receptors. It has been debated whether these compounds are able to block the paracrine and autocrine actions of the membrane bound form of TNF-α. Furthermore, infliximab does not block the actions of LT-α, which is also capable of stimulating the TNF receptors.

Furthermore, both currently available treatments are characterized by relatively long biological half-lives, making them suitable for treatment of chronic diseases. This property is, however, undesirable in connection with treatment of acute disorders such as myocardial infarction and post-traumatic brain damage, where a brief blockade of the destructive effects of TNF may be advantageous while a longer lasting effect may be undesired.

WO 96/40774 discloses lymphotoxin complexes capable of acting as inhibitors of receptors of the tumour necrosis factor (TNF) family. The lymphotoxin complexes are produced by trimerization of separately expressed and purified lymphotoxin monomers. The preparation of said complexes is very laborious and it is difficult to obtain substantially homogenous preparations.

It would thus be useful to develop alternative inhibitors for TNF-R which are easier to produce and which may be useful in treatment of immune reactions and other diseases and conditions such as those listed above.

TNF-α Receptor-Binding Site

As stated above, at least two different TNF receptors are known, these receptors being termed TNF-R1 and TNF-R2. The relative requirements of the TNF receptors TNF-R1 and TNF-R2 in mediating different TNF elicited functions (apoptosis, NF-kB transcription) are still unclear. However, single chain antagonists specific for TNF-R1 or TNF-R2 may be of interest and thus, in a further embodiment the single-chain oligomeric polypeptide of the invention is one which is selective for only one of the receptors, i.e. TNF-R1 or TNF-R2.

In this case, the polypeptide may comprise at least one receptor-binding site with enhanced affinity for binding to a ligand-binding domain of either TNF-R1 or TNF-R2 relative to a corresponding receptor-binding site of a corresponding unmodified single-chain trimeric polypeptide, and at least one inactive receptor-binding site incapable of effectively binding to a ligand-binding domain of either TNF-R1 or TNF-R2, thereby inhibiting and/or activating each TNF-receptor selectively.

For instance, it is known (van Ostade et al., Nature, Vol. 361, pp 266-269, 1993) that each of the mutations L29S and R32W in human TNF-α have lost most of their binding affinity to the TNF-R2, but retained affinity to the TNF-R1. Thus, an example of a single-chain oligomeric polypeptide of the invention is one which comprises one, two or three TNF-α monomers, wherein at least one TNF-α monomer is modified in position L29 and/or R32, preferably by substitution to any other amino acid residue, in particular to S or W, whereby a receptor-binding site comprising said amino acid modification is capable of binding to TNF-R1, but incapable of effectively binding to TNF-R2. For instance, when the single-chain oligomeric polypeptide comprises one TNF-α monomer, this monomer may comprise mutation L29S and/or R32W, when it comprises two TNF-α monomers, one or both of these may comprise mutation L29S and/or R32W, and when it comprises three TNF-α monomers, one, two or all of these may comprise mutation L29S and/or R32W.

Furthermore, when the single-chain oligomeric polypeptide has two active receptor-binding sites both of these may be selective for only one of these receptors, e.g. both may be selective for the same receptor. Alternatively, one receptor-binding site may be selective for one of the receptors, whereas the other may be non-selective. In the Materials and Methods section below, biochemical and cell-based assays are described that may be used to differentiate between the two species of TNF receptor. In another alternative, the single-chain oligomeric polypeptides of the invention may be non-selective, i.e. recognize and bind to either TNF-R1 or TNF-R2.

Also, one receptor-binding site may be selective for a TNF-R, e.g. TNF-R1 and/or TNF-R2, and another receptor-binding site may be selective for a non-TNF receptor recognized by another member of the TNF-family of cytokine ligands. For instance, said other receptor-binding site may be selective towards any of the cytokine ligand receptors mentioned in the Background section above.

In order to obtain a well-functioning antagonist it is preferred that each of the three monomers comprised in a single-chain trimeric TNF polypeptide of the invention is derived from a cytokine ligand of the TNF family, since such ligand is most likely to function as a TNF-R antagonist.

The term “cytokine ligand” refers to a protein released by one cell type or population which acts on at least one other cell type or population to mediate intercellular signalling. Cytokine ligands belonging to the TNF family are described in the Background section above. Within the TNF family, even the most related ligands, TNF-α, LT-α and LT-β are only about 20-30% identical at the amino acid level. Localized sequence comparisons between the TNF ligand family reveal several regions of homology comprising conserved amino acid residues which serve as hallmark features for identifying TNF-related ligands.

Presently known cytokine ligand monomers of the TNF family include the monomer forms of TNFα, LT-α, LT-β, FasL, CD40L, CD30L, CD27L, Ox40L, 4-1BBL, RANKL, TRAIL, TWEAK, LIGHT, TRANCE, APRIL, THANK and TALL-1 (see, e.g., A. Mukhopadhyay, J. Biol. Chem. 1999, 274:25978-81).

However, since new cytokine ligands of the TNF family may be found, such ligands and monomers thereof are contemplated to be equally applicable for the purpose of the present invention as long as they are capable of being modified so as to be part of a single-chain trimeric polypeptide TNF-receptor antagonist as described herein.

While all of the above ligands may be useful in the present invention, it is presently preferred that a single-chain trimeric TNF polypeptide of the invention comprises at least one TNF-α monomer or at least one LT-α monomer, since these monomers constitute part of a natural TNF-R ligand. Furthermore, it may of interest that the single-chain trimeric polypeptide comprises at least one further monomer which is different from TNF-α and LT-α, since a receptor-binding site comprising amino acid residues of such monomer is likely to be inactive, e.g. by being incapable of effectively binding TNF-R, and thus any further modification of amino acid residues of such site can be avoided. In this case, however, it may be necessary to modify amino acid residues of another receptor-binding site of the single-chain trimeric polypeptide so as to increase the binding affinity between said site and TNF-R and thus ensure binding to, but not activation of a TNF-R by the single-chain trimeric polypeptide.

Alternatively or additionally, a single-chain trimeric TNF polypeptide of the invention may comprise at least one monomer derived from a cytokine ligand of the TNF family other than TNF-α. For instance, the polypeptide may comprise three monomers derived from the same or different cytokine ligands of the TNF family other than TNF-α.

When a single-chain trimeric TNF polypeptide of the invention comprises at least one TNF-α monomer, it may comprise one TNF-α monomer and two monomers derived from the same or different cytokine ligand(s) of the TNF family other than TNF-α, two TNF-α monomers and one monomer derived from another cytokine ligand of the TNF family, or three TNF-α monomers, one or more of the monomers being modified so that at least one receptor-binding site of the single-chain trimeric polypeptide is rendered inactive. It will be understood by persons skilled in the art that the same principle applies equally to other oligomeric polypeptides according to the invention, i.e. the monomers chosen for use in a given oligomeric polypeptide may in general be chosen from monomers belonging to the same polypeptide family as the polypeptide whose action the antagonist is intended to block, e.g. the same cytokine family.

Of particular interest with regard to a TNF-α antagonist is a polypeptide wherein the cytokine ligand of the TNF family other than TNF-α is selected from the group consisting of LT-α and LT-β. For instance, said polypeptide may comprise a) one LT-α monomer, one LT-β monomer and one TNF-α monomer; b) two LT-α monomers and one LT-β monomer, c) two LT-α monomers and one TNF-α monomer, d) two LT-β monomers and one LT-α monomer; e) two LT-β monomers and one TNF-α monomer; f) three LT-α monomers; or g) three LT-β monomers. In such cases, the individual monomers may be arranged in any suitable order, so that each of the first, second and third monomers in a single-chain trimeric polypeptide may be independently chosen among, e.g., monomers derived from TNF-α, LT-α and LT-β.

When three LT-β monomers are used, the resulting single-chain trimeric polypeptide is modified so as to replace at least one of the receptor-binding sites with an active TNF-R receptor-binding site, e.g. by substituting one or more amino acid residues forming part of the LT-β receptor-binding site with amino acid residues occupying an equivalent position in LT-α or TNF-α so as to obtain a receptor-binding site which is capable of binding to a TNF-R.

In order to ensure proper folding of a single-chain hetero-trimeric TNF polypeptide of the invention it may be necessary to modify an amino acid residue of an association domain of one or more of the monomers. Accordingly, the single-chain TNF polypeptide may be a heterotrimeric molecule wherein one, two or all of the monomers of the single-chain trimeric polypeptide is/are modified in an association domain thereof so as to enable association of the monomers into an active single-chain trimeric polypeptide with TNF-R antagonist activity. A suitable method of determining association domains is given in the Materials and Methods section herein, as are the amino acid residues forming part of said domains in TNF-α, LT-α and LT-β, respectively.

Since LT-α and LT-β monomers can form homotrimers and mixed heterotrimers in their native environment, the monomer association domains of these ligands, or one or more amino acid residues of said domains, are useful templates for modification of an association domain of a non-LT-α or non-LT-β monomeric polypeptide constituent, such as a TNF-α monomer, which in the single-chain oligomeric polypeptide of the invention is to associate to an LT-α or LT-β monomer. Accordingly, the single-chain trimeric TNF polypeptide may be one wherein at least one non-LT-α or non-LT-β monomer, such as a TNF-α monomer, is modified by substitution of one or more amino acid residues of an association domain thereof for one or more amino acid residues occupying an equivalent position in an association domain of LT-α or LT-β, whereby the resulting modified association domain is capable of associating to an LT-α or an LT-β monomer. In one embodiment the entire TNF-α monomer association domain is replaced by the association domain of LT-α or LT-β. In a further embodiment, the trimeric polypeptide in addition comprises at least one LT-α or LT-β monomer, in particular at least one LT-α monomer, such as two LT-α monomers. In a preferred embodiment, the single-chain trimeric polypeptide is made so as to mimic any of the specific TNF-R antagonists described in WO 96/40774.

In a further specific embodiment, a single-chain TNF polypeptide of the invention is one which comprises at least one TNF-α monomer and at least one monomer derived from a cytokine ligand of the TNF family other than TNF-α, which latter monomer is modified so as to enable association to the TNF-α monomer. Preferably, the monomer is modified by substitution of amino acid residues of the monomer association domain thereof so as to resemble the monomer association domain of TNF-α, e.g. by substitution for one or more amino acid residues occupying an equivalent position in an association domain of TNF-α. This ensures proper folding of the oligomeric polypeptide and consequent activity for the present purpose. For instance, the entire monomer association domain of said latter monomer may be replaced with that of TNF-α.

It will be understood that the modification of an association domain should be performed so as to not significantly disrupt the receptor-binding capability of a receptor-binding site to which said monomer contributes, except where it is desirable that such receptor-binding site is inactivated.

Partial Agonism

It is known in the art that a molecule that binds to a receptor requiring binding of multiple ligands in order to mediate receptor activation, for example the TNF receptors, can have varying degrees of antagonistic and/or agonistic effects depending on the nature of how the individual ligands bind to the ligand-binding domains. In the case of TNF-α for example, a wild-type trimeric polypeptide that binds all three ligand-binding domains will have a purely agonistic effect, i.e. binding of the trimeric polypeptide serves to fully activate the receptor. On the other hand, a trimeric polypeptide with e.g. a single receptor-binding site that strongly binds one of the ligand-binding domains, but which is incapable of binding the other two ligand-binding domains and which blocks these other two domains, thereby effectively preventing receptor activation, will be essentially a pure antagonist without any agonist effect. In between these two extremes, different polypeptides can have different degrees of antagonistic and agonistic effects, so that in many cases a TNF receptor antagonist can have a larger or smaller degree of agonistic effect, concomitant with some degree of antagonistic effect. In such cases, the presence of the TNF receptor antagonist will have an antagonist effect in that binding of wild-type TNF to the TNF receptor will be reduced, as will the effect of the wild-type TNF, but binding of the TNF receptor antagonist to the receptor may in itself result in a certain limited receptor activation. The effects of high levels of TNF may thus be prevented without completely eradicating TNF signalling, i.e. mimicking the natural state of the organism.

In one embodiment, a receptor antagonist of the invention, e.g. a TNF receptor antagonist, has substantially no agonist effect on at least one receptor. Preferably, a TNF receptor antagonist has substantially no agonist effect on either of the TNF receptors TNF-R1 and TNF-R2. This may be obtained by means of a TNF receptor antagonist polypeptide that binds strongly to one or two ligand-binding domains of a TNF receptor and which is unable to bind the other binding domain(s). More preferably, such a TNF receptor antagonist has a single active receptor-binding site that strongly binds a ligand-binding domain of a TNF receptor, and two inactive receptor-binding sites that prevent receptor oligomerisation. The term “substantially no agonist effect” in the present context refers to a maximum stimulation in the luciferase activity assay described herein of not more than about 25% of the maximum stimulation of a reference polypeptide (typically wild-type human TNFα), preferably not more than about 20%, more preferably not more than about 15%, most preferably not more than about 10%, such as not more than about 5%.

In another embodiment, the TNF receptor antagonist may act as a partial antagonist and at the same time as a partial agonist with respect to at least one of the TNF receptors, i.e. resulting in a certain degree of TNF signalling as explained above, but at a substantially lower level than would be the case in the absence of the TNF receptor antagonist.

Amino Acid Residue Modifications of TNF-α

In a specific embodiment, a receptor-binding site of TNF-α is substituted by a receptor-binding site of a cytokine ligand of the TNF family other than TNF-α, e.g. the relevant amino acid residues of a TNF-α receptor-binding site are replaced by amino acid residues of the receptor-binding site of the non-TNF-α cytokine ligand so as to obtain a substantial, e.g. at least 70%, such as at least 80% or at least 90% amino acid identity between amino acid residues of the thus modified receptor-binding site and that of the non-TNF-α cytokine ligand.

In a further specific embodiment the single-chain oligomeric polypeptide of the invention comprises a first, second and third monomer of a cytokine ligand belonging to the TNF family, optionally in truncated form, which monomers are linked, optionally through a peptide linker, so as to form a trimer, which polypeptide comprises a first, a second and a third receptor-binding site located at interfaces between the monomers, wherein

(a) said first and/or second monomer is modified in an amino acid residue constituting part of the first receptor-binding site, and/or wherein

(b) said second and/or third monomer is modified in an amino acid residue constituting part of the second receptor-binding site, and/or wherein

(c) said third and/or first monomer is modified in an amino acid residue constituting part of the third receptor-binding site,

such that the polypeptide comprises one or two active receptor-binding sites capable of binding to a ligand-binding domain of a TNF-R and one or two inactive receptor-binding sites. The first, second and third monomer may be identical (i.e. derived from the same cytokine ligand) or different, and may e.g. be selected from the monomer combinations a)-g) defined above wherein one or more monomers are derived from cytokine ligands of the TNF family other than TNF-α. Alternatively, all three monomers may be TNF-α monomers. The modification is preferably achieved by deletion, substitution and/or insertion of one or more amino acid residues at one or more positions of a receptor-binding site. In order to ensure inactivation of a given receptor-binding site, it is preferred that amino acid residues of the receptor-binding site and originating from both monomers are modified.

In a specific embodiment of a single-chain trimeric polypeptide of the invention comprising three TNF-α monomers, A) said first TNF-α monomer is modified by substitution of one or more amino acid residues in positions E53, S71, T72, H73, V74, L75, T77, R82, A84, V85, S86, Y87, Q88, T89, V91, S95, I97, Q125, E127, N137, or R138, and/or said second TNF-α monomer is modified by substitution of one or more amino acid residues in positions R6, V17, P20, Q21, E23, L29, N30, R31, R32, A33, N34, A35, L63, K65, G66, Q67, E110, A111, K112, P113, W114, Y115, D140, L142, D143, F144, A145, E146, S147 or Q149; B) said second TNF-α monomer is modified by substitution of one or more amino acid residues in positions E53, S71, T72, H73, V74, L75, T77, R82, A84, V85, S86, Y87, Q88, T89, V91, S95, I97, Q125, E127, N137 or R138, and/or said third TNF-α monomer is modified by substitution of one or more amino acid residues in positions R6, V17, P20, Q21, E23, L29, N30, R31, R32, A33, N34, A35, L63, K65, G66, Q67, E110, A111, K112, P113, W114, Y115, D140, L142, D143, F144, A145, E146, S147 or Q149; or C) said third TNF-α monomer is modified by substitution of one or more amino acid residues in positions E53, S71, T72, H73, V74, L75, T77, R82, A84, V85, S86, Y87, Q88, T89, V91, S95, I97, Q125, E127, N137 or R138, and/or said first TNF-α monomer is modified by substitution of one or more amino acid residues in positions R6, V17, P20, Q21, E23, L29, N30, R31, R32, A33, N34, A35, L63, K65, F66, Q67, E110, A111, K112, P113, W114, Y115, D140, L142, D143, F144, A145, E146, S147, or Q149.

In a still further embodiment the polypeptide comprises at least one, such as 1-3 of the mutations mentioned in A) in combination with at least one, such as 1-3 of the mutations mentioned in B); at least one, such as 1-3 of the mutations mentioned in A) in combination with at least one, such as 1-3 of the mutations mentioned in C); or at least one, such as 1-3 of the mutations mention in B) in combination with at least one, such as 1-3 of the mutations mentioned in C).

In a still further embodiment a first TNF-α monomer is modified by substitution of one or more amino acid residues in positions E53, S71, T72, L75, T77, V85, S86, Y87, Q88, T89, V91, 197, Q125, E127, N137 or R138, and/or the subsequent TNF-α monomer is modified by substitution of one or more amino acid residues in positions R6, V17, P20, Q21, E23, L29, N30, R31, R32, A33, K65, Q67, A111, P113, Y115, D143, A145, E146 or S147.

The term “subsequent monomer” is intended to indicate the monomer which forms an interface with the first monomer, which interface comprises a receptor-binding site comprising the specified amino acid residues of the first and subsequent monomer. It will be understood that the term “first monomer” in this context indicates any of the TNF-α monomers of the single-chain trimeric polypeptide. Since the single-chain trimeric polypeptide comprising three TNF-cc monomers is symmetrical it will be understood that the above mutation scheme (as well as any other single-chain trimeric TNF-α mutations disclosed herein) is equally applicable for introduction into a second and/or third monomer and a third and/or first monomer (the first monomer being “subsequent” to the third monomer).

The term “is modified” is used to indicate that the TNF-α monomer comprises a substitution of at least one amino acid residue occupying the indicated position. Thus, the TNF-α monomer may additionally comprise other substitutions not mentioned in the above list.

More preferably, a first TNF-α monomer is modified by substitution of one or more amino acid residues in positions E53, S71, T72, L75, T77, V85, S86, Y87, Q88, T89, V91, I97, Q125 or E127, and/or the subsequent TNF-α monomer is modified by substitution of one or more amino acid residues in positions R6, V17, P20, Q21, R31, R32, K65, Q67, A111, P113, Y115, D143, A145, E146 or S147.

Still more preferably, a first TNF-α monomer is modified by substitution of one or more amino acid residues in positions E53, S71, L75, T77, S86, Y87, T89, V91, I97, Q125 or E127, and/or the subsequent TNF-α monomer is modified by substitution of one or more amino acid residues in positions V17, P20, R31, R32, K65, Y115, D143, A145 or E146.

Still more preferably, a first TNF-α monomer is modified by substitution of one or more amino acid residues in positions E53, S71, L75, T77, S86, Y87, T89, V91, I97, or E127, and/or the subsequent TNF-α monomer is modified by substitution of one or more amino acid residues in positions V17, P20, R31, R32, K65, Y115, D143, A145 or E146.

Still more preferably, a first TNF-α monomer is modified by substitution of one or more amino acid residues in positions L75, Y87, V91 or 197, and/or the subsequent TNF-α monomer is modified by substitution of one or more amino acid residues in positions P20, R32, K65, Y115, D143, A145 or E146. For instance, a first TNF-α monomer is modified by substitution of one or more amino acid residues in positions L75, Y87, V91 or I97, and/or the subsequent TNF-α monomer is modified by substitution of one or more amino acid residues in positions P20, R32, K65, Y115, D143 or E146; or a first TNF-α monomer is modified by substitution of one or more amino acid residues in positions L75, Y87 or V91, and/or the subsequent TNF-α monomer is modified by substitution of one or more amino acid residues in positions P20, K65, Y115, D143, A145 or E146.

Still more preferably, a first TNF-α monomer is modified by substitution of one or more amino acid residues in positions L75, Y87 or V91, and/or the subsequent TNF-α monomer is modified by substitution of one or more amino acid residues in positions P20, Y115, D143 or E146. For instance, a first TNF-α monomer is modified by substitution of one or more amino acid residues in positions L75, Y87 or V91, e.g. L75 and V87; L75 and V91; Y87 and V91; or L75, Y87 and V91, and optionally the subsequent TNF-α monomer is modified in position Y115 and/or D143.

In any of the above listed modifications, it is preferred that the substitution be in accordance with Table 1 above. Thus, for instance, when L75 is to be substituted this is preferably to any of G, A, S, T, D, N, E, Q, H, Y, R and K.

In another embodiment, a receptor-binding site of a single-chain trimeric polypeptide of the invention comprising at least one TNF-α monomer is inactivated by modification of said TNF-α monomer in position A33, e.g. by one of the substitutions A33Y or A33R; in position 84, e.g. by the substitution to any of the relevant amino acid residues apparent from Table 1 above, or by the substitution A84V, in position 86, e.g. by the substitution S86D or S86R, in position S95, e.g. by the substitution S95Y; in position P113, e.g. by the substitution P113D; in position Y115, in positions D143 and A145, e.g. by the substitutions D143R+A145Y; in positions R32 and A33, e.g. by the substitutions R32E+A33Y; or in positions S86 and Y87, e.g. by the substitutions S86D+Y87R.

In a preferred embodiment, the single-chain oligomeric polypeptide of the invention is one wherein the modification of a first and a subsequent TNF-α monomer consists of at least one of the mutations specified above.

Glycosylation of Single-Chain TNF-α

As explained above, the single-chain oligomeric polypeptides of the invention may be modified to provide steric hindrance by means of attachment of a non-polypeptide moiety, e.g. an oligosaccharide.

Steric hindrance may e.g. be obtained by N-glycosylation at an N-glycosylation site which has been introduced into the amino acid sequence in such a manner that the resulting polypeptide, when expressed in a glycosylating host cell, comprises an oligosaccharide moiety which is positioned so as to render one or two receptor-binding sites inactive.

For instance, a single-chain trimeric polypeptide of the invention is one which comprises three TNF-α monomers, and wherein a glycosylation site has been introduced into a receptor-binding site located in the interface between a first and a second monomer. More specifically, a first TNF-α monomer comprises one or more of the substitutions E53N+L55S, E53N+L55T, S71N+H73S, S71N+H73T, T72N+V74S, T72N+V74, H73N+L75S, H73N+L75T, V74N+L76S, V74N+L76T, L75N+T76S, L75N, T77N+T79S, T77N, R82N+A84S, R82N+A84T, A84N, A84N+S86T, V85N+Y87S, V85N+Y87T, S86N+Q88S, S86N+Q88T, Y87N+T89S, Y87N, Q88N+K90S, Q88N+K90T, T89N+V91S, T89N+V91T, Q125N+E127S, Q125N+E127T, E127N+G129S, E127N+G129T, P139S, and P139T, and/or the subsequent TNF-α monomer comprises one or more of the substitutions R6N+P8S, R6N+P8T, P20N+A22S, P20N+A22T, Q21N+E23S, Q21N+E23T, E23N+Q25S, E23N+Q25T, L29N+R31S, L29N+R31T, R32S, R32T, R31N+A33S, R31N+A33T, R32N+N34S, R32N+N34T, A33N+A35S, A33N+A35T, L36S, L36T, A35N+L37S, A35N+L37T, L63N+K65S, L63N+K65T, K65N+Q67S, K65N+Q67T, G66N+G68S, G66N+G68T, A111N+P113S, A111N+P113T, P113N+Y115S, P113N+Y115T, Y115N+P117S, Y115N+P117T, D140N+L142S, D140N+L142T, L142N+F144S, L142N+F144T, D143N+A145S, D143N+A145T, F144N+E146S, F144N+E146T, A145N+S147S, A145N+S147T, E146N+G148S, E146N+G148T, S147N+Q149S, S147N+Q149T, Q149N+Y151S and Q149N+Y151T.

Preferably, a first TNF-α monomer comprises one or more of the substitutions E53N+L55S, E53N+L55T, S71N+H73S, S71N+H73T, T72N+V74S, T72N+V74, L75N+T76S, L75N, T77N+T79S, T77N, V85N+Y87S, V85N+Y87T, S86N+Q88S, S86N+Q88T, Y87N+T89S, Y87N, Q88N+K90S, Q88N+K90T, T89N+V91S, T89N+V91T, Q125N+E127S, Q125N+E127T, E127N+G129S, E127N+G129T, P139S and P139T, and/or the-subsequent TNF-α monomer comprises one or more of the substitutions R6N+P8S, R6N+P8T, P20N+A22S, P20N+A22T, Q21N+E23S, Q21N+E23T, E23N+Q25S, E23N+Q25T, L29N+R31S, L29N+R31T, R32S, R32T, R31N+A33S, R31N+A33T, R32N+N34S, R32N+N34T, A33N+A35S, A33N+A35T, K65N+Q67S, K65N+Q67T, A111N+P113S, A111N+P113T, P113N+Y115S, P113N+Y115T, Y115N+P117S, Y115N+P117T, D143N+A145S, D143N+A145T, A145N+S147S, A145N+S147T, E146N+G148S, E146N+G148T, S147N+Q149S and S147N+Q149T.

Still more preferably, a first TNF-α monomer comprises one or more of the substitutions E53N+L55S, E53N+L55T, S71N+H73S, S71N+H73T, L75N+T76S, L75N, T77N+T79S, T77N, S86N+Q88S, S86N+Q88T, Y87N+T89S, Y87N, T89N+V91S, T89N+V91T, Q125N+E127S, Q125N+E127T, E127N+G129S and E127N+G129T, and/or the subsequent TNF-α monomer comprises one or more of the substitutions P20N+A22S, P20N+A22T, R31N+A33S, R31N+A33T, R32N+N34S, R32N+N34T, K65N+Q67S, K65N+Q67T, Y115N+P117S, Y115N+P117T, D143N+A145S, D143N+A145T, A145N+S147S, A145N+S147T, E146N+G148S and E146N+G148T.

For instance, a first TNF-α monomer may comprise one or more of the substitutions L75N+T76S, L75N, Y87N+T89S and Y87N, and/or the subsequent monomer may comprise one or more of the substitutions P20N+A22S, P20N+A22T, R32N+N34S, R32N+N34T, K65N+Q67S, K65N+Q67T, D143N+A145S, D143N+A145T, A145N+S147S, A145N+S147T, E146N+G148S and E146N+G148T; or a first TNF-α monomer may comprise one or more of the substitutions L75N+T76S, L75N, Y87N+T89S and Y87N, and/or the subsequent TNFα monomer may comprise one or more of the substitutions P20N+A22S, P20N+A22T, D143N+A145S, D143N+A145T, E146N+G148S and E146N+G148T.

Most preferably, a first TNF-α monomer comprises or consists of one or more of the substitutions L75N, Y87N and V91N+L93T, and/or the subsequent monomer one or more of the substitutions P20N+A22T, D143N+A145T, and E146N+G148T.

In a preferred embodiment, the single-chain oligomeric polypeptide is one wherein the modification of a first and a subsequent TNF-α monomer consists of at least one, and preferably two of the sets of mutations specified above so as to have incorporated at least one, and preferably two or more glycosylation sites.

In some cases it may be an advantage to alter the receptor-binding site in a manner resulting in a site that shows a stronger binding affinity to one or both of TNF-R1 and TNF-R2 than the wild-type site. The invention therefore provides for a single-chain oligomeric polypeptide comprising at least one receptor-binding site with increased binding affinity to a ligand-binding domain of said TNF receptor. The increased binding affinity may be selective towards one of the TNF-receptors, such as TNF-R1 or TFN-R2, or both of these.

In one embodiment, it is preferred that the polypeptide of the invention has a higher binding affinity towards a ligand-binding domain of a TNF receptor relative to wild-type human TNF-α. The higher binding affinity is conveniently determined as described in the Materials and Methods section herein. Alternatively, the higher binding affinity to a ligand binding domain of a TNF receptor is relative to a corresponding receptor-binding site of a corresponding unmodified single-chain oligomeric polypeptide. For instance, when the single-chain oligomeric polypeptide comprises three TNF-α or LT-α monomers, the relevant comparison is to a corresponding receptor-binding site of wild-type TNF-α or LT-α.

In addition to having at least one inactive receptor-binding site, the single-chain oligomeric polypeptide of the invention may have one or two receptor-binding sites with a higher binding affinity towards a ligand-binding domain of a TNF receptor relative to a corresponding receptor-binding site of a corresponding unmodified single-chain oligomeric polypeptide. The higher binding affinity is suitably obtained by substitution or insertion of one or more amino acid residues in said receptor-binding site so as to obtain a stronger binding affinity to said receptor. Suitable amino acid substitutions may be conservative substitutions as described herein.

In a more specific embodiment, the single-chain polypeptide of the invention comprises three TNF-α monomers that are modified so as to increase the binding affinity of at least one receptor-binding site towards a ligand-binding domain of a TNF-receptor. In the polypeptide according to this embodiment, a first TNF-α monomer is modified by substitution of one or more amino acid residues in positions E53, S71, T72, H73, V74, L75, T77, R82, A84, V85, S86, Y87, Q88, T89, V91, I97, Q125, E127, N137 and R138; and/or the subsequent monomer is modified by substitution of one or more amino acid residues in positions R6, V17, P20, Q21, E23, L29, N30, R31, R32, A33, N34, A35, L63, K65, G66, Q67, E110, A111, K112, P113, W114, Y115, D140, L142, D143, F144, A145, E146, S147 and Q149.

More preferably, a first TNF-α monomer is modified by substitution of one or more amino acid residues in positions E53, S71, T72, L75, T77, V85, S86, Y87, Q88, T89, V91, I97, Q125, E127, N137 and R138, and/or the subsequent TNF-α monomer is modified by substitution of one or more amino acid residues in positions R6, V17, P20, Q21, E23, L29, N30, R31, R32, A33, K65, Q67, A111, P113, Y115, D143, A145, E146 and S147.

Even more preferably, a first TNF-α monomer is modified by substitution of one or more amino acid residues in positions E53, S71, T72, L75, T77, V85, S86, Y87, Q88, T89, V91, I97, Q125 and E127, and/or the subsequent monomer is modified by substitution of one or more amino acid residues in positions R6, V17, P20, Q21, R32, K65, Q67, A111, P113, Y115, D143, A145, E146 and S147.

Even more preferably, a first TNF-α monomer is modified by substitution of one or more amino acid residues in positions E53, S71, L75, T77, S86, Y87, Q88, T89, V91, I97, Q125 and E127, and/or the subsequent monomer is modified by substitution of one or more amino acid residues in positions V17, P20, Q21, R32, K65, Y115, D143, A145 and E146.

Still more preferably, a first TNF-α monomer is modified by substitution of one or more amino acid residues in positions E53, S71, L75, T77, S86, Y87, Q88, T89, V91, I97 and E127, and/or the subsequent monomer is modified by substitution of one or more amino acid residues in positions V17, P20, Q21, R32, K65, Y115, D143, A145 and E146.

For instance, a first TNF-α monomer may be modified by substitution of one or more amino acid residues in positions E53, S71, L75, T77, S86, Y87, Q88, T89, V91, I97 and E127, and/or the subsequent monomer may be modified by substitution of one or more amino acid residues in positions V17, P20, Q21, R32, K65, Y115, D143, A145 and E146.

Still more preferably, a first TNF-α monomer may be modified by substitution of one or more amino acid residues in positions S71 and/or I97, and/or the subsequent monomer may be modified by substitution of one or more amino acid residues in positions P20, Q21, R32, K65, Y115, D143 and A145, in particular P20, Q21, Y115 and/or D143.

For instance, a first TNF-α monomer may be modified by substitution of amino acid residue I97, and/or the subsequent monomer may be modified by substitution of one or more amino acid residues in positions P20, Q21, Y115 and D143.

Alternatively, a first TNF-α monomer may be unmodified and the subsequent monomer may be modified by substitution of one or more amino acid residues in positions Q21, Y115 and D143.

Specific examples of the modifications described above include modification of a first TNF-α monomer by the substitution S71R and/or I97E and/or modification of the subsequent monomer by one or more of the following substitutions P20D, Q21E, R32Q, Y115K, D143Q and D143E. For instance, the first TNF-α monomer may comprise the mutation I97E and/or the subsequent monomer the mutation Y115K.

VEGF

VEGF: Background

The invention may be further illustrated by a VEGF polypeptide comprising a first and second VEGF monomer covalently linked so as to form a dimer, wherein the first and second VEGF monomer are modified in at least one position within a receptor-binding site thereof such that the polypeptide comprises one intact receptor-binding site capable of binding to a ligand-binding domain of a VEGF receptor and one modified receptor-binding site which is incapable of effectively binding to a ligand-binding domain of a VEGF receptor.

VEGF and Angiogenesis

Angiogenesis is the sprouting of blood capillaries from pre-existing blood vessels, while vasculogenesis is the de novo development of blood vessels through differentiation of early endothelial cells during embryonic development.

VEGF is a mitogen that is highly specific for vascular endothelial cells (Dvorak et al. (1995), Am. J. Pathol. 146, 1029-1039). VEGF is a potent angiogenic/vasculogenic factor involved in the development of the vascular system and in the differentiation of endothelial cells as shown by the lethality of targeted disruption of even one allele of the VEGF gene (Carmeliet et al. (1996), Nature 380, 435-439; Ferrara et al. (1996), Nature 380, 439-442). VEGF is sometimes also called vascular permeability factor (VPF).

Angiogenesis/vaculogenesis is, however, not only important in the physiological processes of embryogenesis and wound healing. It is also involved in pathological processes such as tumour growth, metastasis, diabetic retinopathy and rheumatoid arthritis. It is for instance well established that tumour microvessel density and vascular permeability influence the prognosis in various forms of cancer with a good correlation between vascularisation, metastasis, malignancy and survival rates.

An important role for VEGF as a mediator of tumour angiogenesis is suggested by a number of observations. High levels of VEGF are produced by various types of tumours with the result that capillaries are clustered along VEGF-producing tumour cells, and it has been found that VEGF expression/overexpression correlates well with the induction of neovascularisation in tumours in a number of different cancers and in many cases also with a poor prognosis. It has also been shown that tumour angiogenesis and subsequent tumour growth are inhibited in vivo when VEGF signalling is inhibited by various means.

Thus, there is a large body of evidence showing that neutralising the action of VEGF results in an inhibition of tumour angiogenesis and an inhibition of tumour growth.

The VEGF Molecule

At present, five different human VEGF mRNA species have been identified coding for VEGF isoforms containing 121, 145, 165, 189, and 206 amino acid residues, respectively (Leung et al. (1989), Science 246, 1306-1309; Keck et al. (1989), Science 246, 1309-1312; Tischeret al., (1991), J. Biol. Chem. 266, 11947-11954; Houcket al. (1991), Mol. Endocrinol. 5, 1806-1814; Poltorak et al. (1997), J. Biol. Chem. 272, 7151-7158). VEGF₁₆₅ is the most abundantly expressed isoform followed by VEGF₁₂₁ as the second most abundantly expressed isoform. VEGF₁₂₁ may be considered a functional fragment of any of the other isoforms and the four first mentioned isoforms may be considered functional fragments of the VEGF₂₀₆ isoform. The five VEGF mRNA species are most likely produced by alternative splicing of exons 1-5, 6a, 6b, 7 and 8. All five isoforms share a common N-terminal region of 115 amino acid residues (encoded by exons 1-5) and the six C-terminal amino acid residues encoded by exon 8. The three longest isoforms share the same 50 C-terminal residues.

All the VEGF isoforms are bioactive. Thus, not unexpectedly, it is the common N-terminal region of 115 amino acid residues shared by all five isoforms that contains the structural information required for recognition by and binding to the two VEGF-receptors (see below) (Keyt et al. (1996), J. Biol. Chem. 271, 7788-7795). This has been clearly shown as 110 amino acid residue N-terminal fragments of VEGF₁₆₅ and VEGF₁₈₉, generated through cleavage by the protease plasmin, have been shown to be endothelial cell mitogens, and to bind to both VEGF-receptors (Keyt et al., supra; Houck et al. (1992), J. Biol. Chem. 267, 26031-26037). The five isoforms differ with respect to binding to heparin, heparan sulfate, and the extracellular matrix (ECM).

In summary, the gene for VEGF contains coding sequences for domains/regions that confer receptor-binding, heparin/ECM-binding, and heparin-binding. Differential use of this genetic information results in five VEGF isoforms with different binding capabilities for heparin and the ECM and in consequence with different bioavailability and therefore different bioactivity.

VEGF has been purified from a variety of species as a disulfide-bonded apparently homodimeric protein with a relative molecular weight around 45 kDa as estimated from SDS-PAGE. In accordance with this the monomer (which is biologically inactive) has an estimated molecular weight of 23 kDa.

The amino acid sequence of human VEGF contains one potential N-glycosylation site at Asn75, and studies have shown that glycosylated and non-glycosylated VEGF has the same biological activity.

The disulfide-bonding of the cysteine-residues in VEGF₁₆₅ has been deduced from X-ray crystallography for the N-terminal receptor-binding site (Muller et al. (1997), Proc. Natl. Acad. Sci. USA 94, 7192-7197) and from N-terminal amino acid sequencing of tryptic fragments of the C-terminal heparin-binding domain (Keck et al. (1997), Arch. Biochem. Biophys. 344, 103-113). In the receptor-binding site, the three intra-chain disulfide-bonds Cys26-Cys68, Cys57-Cys102, and Cys6l-Cys104 forms a so-called cystine knot motive (see below). Cys51 and Cys60 are engaged in the two inter-chain disulfide-bonds holding the two anti-parallel monomers covalently together, thus, Cys51 in one monomer forms a disulfide-bond with Cys60 in the other monomer and vice versa. In the heparin-binding domain of VEGF₁₆₅ the four disulfide-bonds are Cyst 17-Cys135, Cys120-Cys137, Cys139-Cys158, and Cys146-Cys160.

Based on the amino acid sequence homology to PDGF, VEGF was included in the superfamily of cystine knot growth factors (Sun et al. (1995), Annu. Rev. Biophys. Biomol. Struct. 24, 269-291). This has further been confirmed by the three-dimensional structure of the receptor-binding site of VEGF. The topology of the VEGF monomer is similar to that observed in PDGF (Oefner et al. (1992), EMBO. J. 11, 3921-3926), although upon alignment of the two amino acid sequences only 19% of the positions are occupied by identical amino acid residues.

The VEGF monomer contains a total of seven β strand segments (β1 to β7) and two α-helical segments (α1 and α2). The most prominent and central feature in the structure of the VEGF monomer is a central highly irregular antiparallel four-stranded α sheet comprising strands β1, β3, β5, and β36. This four-stranded β sheet displays the characteristic cystine knot at one end. The cystine knot consists of two disulfide bonds forming a covalently linked ring structure between two adjacent β strands (β3 and β7) together with a third disulfide bond penetrating this ring and connecting the beginning of two other β strands (β1 and β4).

VEGF dimerizes in an antiparallel side-by-side fashion and the monomers are, as already mentioned, covalently linked by two disulfide bonds. Structural elements at the opposite end of the monomer from the cystine knot are involved in formation of a hydrophobic core across the monomer-monomer subunit interface. The amino acid residues involved in this hydrophobic core are derived from the loop connecting strands β1 and β3, the end of strand β35, the beginning of strand β6, and the loop connecting strands β5 and β36, all from one monomer in combination with the N-terminal α-helix (α1) of the other monomer. As will be described below, this hydrophobic core is important in receptor binding.

The VEGF Receptors

As mentioned above, VEGF is presumably a vascular endothelial cell-specific growth factor as it appears to be inactive on fibroblasts, keratinocytes, vascular smooth muscle cells, lens epithelial cells, corneal endothelial cells, adrenal cortical cells, and granulosa cells (Ferrara et al. (1989), Biochem. Biophys. Res. Commun. 161, 851-858; Gospodarowicz et al. (1989), Proc. Natl. Acad. Sci. USA 86, 7311-7315).

VEGF exerts its effects on vascular endothelial cells through at least two receptors known as Flt-1 (fms-like tyrosine kinase 1, also known as VEGF-receptor 1) and KDR (kinase domain receptor or kinase-insert domain-containing receptor, also known as VEGF-receptor 2). Both receptors are tyrosine kinases.

The amino acid sequences of human Flt-1 (110) as well as of human KDR (Terman et al. (1992), Biochem Biophys. Res. Commun. 187, 1579-1586; Terman et al. (1991), Oncogene 6, 1677-1683) are known and show that both proteins comprising more than 1300 amino acid residues are composed of 7 extracellular immunoglobulin-like (Ig-like) domains containing the ligand-binding domains, a transmembrane domain and an intracellular tyrosine kinase domain. Thus, the two VEGF-receptors are homologous. Overall, identical amino acid residues occupy approximately 40% of the homologous positions, but this percentage is much higher in large parts of the tyrosine kinase domain. A number of studies imply that KDR but not Flt-1 plays the important role in VEGF-induced mitogenesis.

The three-dimensional structure of Ig-like domain 2 from Flt-1 has been determined in complex with the receptor-binding domain of VEGF (residues 8-109) (Wiesmann et al. (1997), Cell 91, 695-704) giving information about the receptor-ligand interactions. The three dimensional structure of a variant of the kinase domain from KDR has also recently been solved (McTigue et al. (1999), Structure Fold. Des. 15, 319-330). Further, a third VEGF-receptor has been identified on endothelial cells and various tumour cells (Gitay-Goren et al. (1992), J. Biol. Chem. 267, 6093-6098; Gitay-Goren et al. (1993), Biochem. Biophys. Res. Commun. 190, 702-708; Gitay-Goren et al. (1996), J. Biol. Chem. 271, 5519-5523; Soker et al. (1996), J. Biol. Chem. 271, 5761-5767; Omura et al. (1997), J. Biol. Chem. 272, 23317-23322). Interestingly, this receptor binds VEGF₁₆₅ but not VEGF₁₂₁, and apparently it does so through the exon 7-encoded domain as this is the only difference between the two proteins (Soker et al. (1996), supra; Soker et al. (1997), J. Biol. Chem. 272, 31582-31588). This third VEGF-receptor has through purification and expression cloning from a tumour cell line been identified as neuropilin-1 (Soker et al. (1998), Cell 92, 735-745). Neuropilin-1 is expected to be a co-receptor for VEGF₁₆₅ which is supported by the observation that KDR binds VEGF₁₆₅ more efficiently in cells expressing neuropilin-1 than in cells not expressing neuropilin-1 (Soker et al. (1998), supra).

The VEGF: VEGF-Receptor Interaction

The interaction between the receptor-binding site of VEGF and the two VEGF-receptors Flt-1 and KDR has been studied in a variety of ways, giving information on different levels.

Many studies have had the aim of identifying the VEGF-binding parts of the VEGF-receptors. Especially the VEGF-binding domain of Flt-1 has been investigated and although minor differences exist among the studies it may be concluded that Ig-like domain 2 from Flt-1 is necessary for binding of VEGF although not sufficient for VEGF-binding with wild type Flt-1 affinity (Wiesmann et al. (1997), supra; Davis-Smyth et al. (1996), EMBO J. 15, 4919-4927; Cunningham et al. (1997), Biochem. Biophys. Res. Commun. 231, 596-599; Barleon et al. (1997), J. Biol. Chem. 272, 10382-10388; Tanaka et al. (1997), Jpn. J. Cancer Res. 88, 867-876; Herley et al. (1999), Biochem. Biophys. Res. Commun. 262, 731-738). The general observation is that Ig-like domains 1-3 bind VEGF with wild type Flt-1 affinity.

For KDR, Ig-like domains 2-3 are sufficient for VEGF-binding with wild type KDR affinity (Fuh et al. (1998), J. Biol. Chem. 273, 11197-11204).

It is interesting to note, however, that the monomer/dimer status of the VEGF-receptors have different significance for the affinity for VEGF of the two receptors. In many of the studies aimed at clarifying the minimal domain-requirements for VEGF-binding by the receptors, the receptor-constructs were expressed as fusion proteins between the receptor-Ig-like-domains in question and parts of the heavy chain from IgG. Through the IgG-part these constructs dimerise and the binding constants determined are thus the binding constants for the interaction between dimeric VEGF and these predimerised VEGF-receptor-constructs. In some studies monomeric VEGF-receptor-constructs have also been made (Fuh et al. (1998), supra; Wiesmann et al. (1997), supra) and here a striking difference between the two VEGF-receptors were found. The difference in VEGF-binding affinity for monomeric and predimerised Flt-1-constructs were found to be minimal (approximately 2-fold weaker VEGF-binding by monomeric than by predimerised Flt-1 constructs) (Wiesmann et al. (1997), supra). For the KDR-constructs a different situation was found as monomeric KDR-constructs have a 100-fold weaker VEGF-binding than predimerised KDR-constructs (Fuh et al. (1998), supra).

The most detailed information about the interactions between VEGF and a VEGF-receptor has been obtained from the three dimensional structure of the complex between the receptor-binding domain of VEGF and the Ig-like domain 2 from Flt-1 (Wiesmann et al. (1997), supra). From the structure it can be seen that the receptor-binding sites in VEGF are at the poles of the dimer and formed at the interface between the VEGF-monomers. The contact surface in the VEGF dimer is divided about 65%/35% between the two monomers. In one monomer, the contact surface involves amino acid residues 16-27 (the N-terminal helix), 61-66 (the loop between strand β3 and β4), and 103-106 (strand β7). In the second monomer, the contact surface involves amino acid residues 46-48 (strand β2) and 79-91 (strand β5-loop-strand β6).

In Flt-1 Ig-like domain 2 the contact surface is comprised by amino acid residues from the N-terminal bulge, strand βa′, part of strands βg and βf, the loop connecting strands βc and βc′, and the helical turn connecting strands βe and βf.

Alanine-scanning mutagenesis has also been attempted previously as a means for elucidating the receptor-binding site in VEGF (Muller et al. (1997), supra; Keyt et al. (1996), J. Biol. Chem. 271, 5638-5646). In the first study, one site in VEGF of major importance for KDR-binding was found to be defined by Arg82, Lys84, and His86 while a site of major importance for Flt-1-binding was found to be defined by Asp63, Glu64, and Glu67. Interestingly, the site of major importance for binding to one receptor was found to be of minor (but detectable) importance for the binding to the other receptor (Keyt et al. (1996), supra). Along the same lines it was observed that introduction of an N-glycosylation site at position 82 in VEGF through the substitutions Arg82Asn, Ile83Leu, Lys84Ser influenced the binding to KDR significantly but not the binding to Flt-1. More elaborate alanine-scanning mutagenesis of VEGF using phage display led to the proposal that the KDR-binding site was defined by amino acid residues Phe17, Ile43, Ile46, Glu64, Gln79, Ile83, lys84, and Pro85 (Muller et al. (1997), supra).

Comparing the results of the alanine-scanning mutagenesis and the conclusions based hereupon with the knowledge obtained from the three dimensional structure of the complex between the receptor-binding domain of VEGF and Ig-like domain 2 from Flt-1, they are not completely in agreement. Of the VEGF amino acid residues that were found to be of moderate to great importance for tight binding to KDR, 5 are buried in the interface with Flt-1 Ig-like domain 2 in the complex, suggesting that the binding sites for KDR and Flt-1 are very similar (Wiesmann et al. (1997), supra). This is not the interpretation based upon the alanine-scanning mutagenesis data alone, as these suggested separate binding sites for Flt-1 and KDR on VEGF. The reason for this discrepancy is not presently known. Nevertheless, it is contemplated that the binding sites for Flt-1 and KDR are indeed somewhat overlapping, and that both of the above techniques may provide valuable information for producing VEGF variants.

VEGF Variants

In two studies heterodimeric VEGF variants with one functional and one non-functional receptor-binding site have been constructed and characterised (Siemeister et al. (1998), Proc. Natl. Acad. Sci. USA 95, 4625-4629; Fuh et al. (1998), supra).

In the first study (Siemeister et al.) two homodimeric VEGF variants were made initially. In the first of these VEGF variants Asp63, Glu64, Gly65, and Leu66 were substituted with Thr63, Ser64, Ser65, and Val66, which are the corresponding residues from PDGF-A. This VEGF variant did not bind to Flt-1, while the affinity for KDR was decreased 4- to 5-fold. In the second of these VEGF variants Arg82, Lys84, Gln87, Gly88, and Gln89 were substituted with Glu82, Glu84, Arg87, Lys88, and Lys89, which are the corresponding residues from PDGF-A/B. This VEGF variant bound Flt-1 with unchanged affinity, while the affinity for KDR was decreased more than 40-fold. These binding data are completely in line with the data obtained using alanine-scanning mutagenesis. Next, a heterodimeric VEGF molecule was made by recombining monomers of the two VEGF variants in vitro. This VEGF heterodimer is supposed to contain one functional and one non-functional receptor-binding site. In accordance with this, it was shown that the VEGF heterodimer binds to Flt-1 with wild type VEGF affinity and to KDR with a slightly decreased affinity. Furthermore, the VEGF heterodimer was shown to be a VEGF-antagonist as it strongly inhibited KDR autophosphorylation and proliferation of human endothelial cells.

In the second study (Fuh et al.), a heterodimeric VEGF molecule was made by recombining monomers of the following two VEGF variants in vitro. One variant contained the substitutions Ile46Ala, Cys51Ser, and Ile83Ala while the other variant contained the substitutions Phe17Ala, Cys60Ser, and Glu64Ala. The reason for the substitutions of the Cys-residues is that this ensures that the only covalently bound dimers will be heterodimers. (Wild type VEGF covalently bound by only one disulfide bond binds KDR with wild type affinity.) The heterodimer bound monomeric KDR only 2-fold weaker than wild type VEGF. It was also shown that the heterodimer did not dimerise and activate KDR, and finally it was shown that the heterodimer antagonises signalling by KDR.

It appears from the above that VEGF-heterodimers with one functional and one non-functional receptor-binding site are antagonists of signalling through the KDR tyrosine kinase. However, the methods previously used to produce the antagonists are clearly suboptimal and laborious. In addition, no real efforts have been put into optimizing the antagonistic effects of various heterodimers, perhaps due to the laborious production methods.

In the studies mentioned above, the VEGF-heterodimers with one functional and one non-functional receptor-binding site are produced by recombining two different VEGF-monomers in vitro in the following way. As the first step, the two different VEGF-monomers were obtained from separate expressions in two E. coli strains each carrying the genetic information to express one VEGF-monomer variant. The VEGF-monomer variants were expressed in inclusion bodies, making re-solubilisation using strong denaturants necessary before purification (and optional re-folding) in order to obtain pure VEGF-variant preparations.

However, in order to obtain VEGF-heterodimers with one functional and one non-functional receptor-binding site, the two different VEGF-monomer variants had to be recombined in vitro. This was carried out by mixing equimolar amounts of the two different VEGF-monomer variants under strongly denaturing conditions followed by re-folding by dialysis. Following this, the final purifications of VEGF-heterodimers were done in order to remove residual VEGF-monomers and possibly formed VEGF-homodimers.

The instant invention presents a different approach to obtaining, e.g., VEGF antagonists by expressing variants of VEGF-heterodimers with one functional and one non-functional receptor-binding site as single-chain polypeptides. This approach has several advantages:

There is no need to express two VEGF constructs as all the genetic information is contained within a single construct.

There is no need to recombine separately expressed VEGF monomers into VEGF-heterodimers as these are expressed as a single polypeptide chain.

There is only need for purification of VEGF-heterodimers.

Furthermore, an elaborate structural analysis of the three-dimensional structure of the complex between the receptor-binding domain of VEGF and Ig-like domain 2 from Flt-1 has been carried out in order to rationalise and optimize the development of VEGF antagonists consisting of VEGF-heterodimers with one functional and one non-functional receptor-binding site.

The single-chain VEGF polypeptides are all of the following overall structure- VEGF-monomer A—peptide bond or linker peptide-VEGF-monomer B, where VEGF-monomer A is selected from variants of VEGF₁₁₀, VEGF₁₂₁, VEGF₁₄₅, VEGF₁₆₅, VEGF₁₈₉, or VEGF₂₀₆; and where VEGF-monomer B is independently selected from variants of VEGF₁₁₀, VEGF₁₂₁, VEGF₁₄₅, VEGF₁₆₅, VEGF₁₈₉, or VEGF₂₀₆. The linker, when present, may be any peptide sequence of less than 25 amino acid residues.

In one general example, one or more substitutions in VEGF-monomer A may be carried out in the region comprising amino acid residues 16-27 (the N-terminal helix), 61-66 (the loop between strand β3 and β4), and 103-106 (strand β7) in combination with one or more substitutions in VEGF-monomer B in the region comprising amino acid residues 46-48 (strand β2) and 79-91 (strand β5-loop-strand β6).

In another general example, one or more substitutions in VEGF-monomer A may be carried out in the region comprised by amino acid residues 46-48 (strand β2) and 79-91 (strand β5-loop-strand β6) in combination with one or more substitutions in VEGF-monomer B in the region comprised by amino acid residues 16-27 (the N-terminal helix), 61-66 (the loop between strand β3 and β4), and 103-106 (strand β7).

In one preferred example, VEGF-monomer A is a variant of VEGF₁₂₁ while VEGF-monomer B is a variant of VEGF₁₆₅.

In another preferred example, VEGF-monomer A is a first variant of VEGF₁₂₁ while VEGF-monomer B is a second, different variant of VEGF₁₂₁.

Based on these observations, the amino acid residues involved in interactions with either receptor subunit may be modified. Residues within 5 Å of the receptor subunit are major targets for modification of the first receptor-binding site:

In VEGF monomer A: Phe17, Met18, Tyr21, Gln22, Tyr25, Asn62, Asp63, Gly65, Leu66, Glu103, Cys104, Arg105 or Pro106.

In VEGF monomer B: Ile46, Lys48, Gln79, Met81, Ile83, Gln89 or Ile91.

More preferred positions for modification in VEGF monomer A: Phe17, Met18, Tyr21, Gln22, Tyr25, Asp63, Gly65, Leu66, Glu103, Arg105 or Pro106.

More preferred positions for modification in VEGF monomer B: Ile46, Lys48, Gln79, Met81, Ile83, Gln89 or Ile91.

Still more preferred positions for modification in VEGF monomer A: Phe17, Met18, Tyr21, Gln22, Tyr25, Asp63, Leu66, Glu103, Arg105 or Pro106.

Still more preferred positions for modification in VEGF monomer B: Ile46, Lys48, Gln79, Met81, Ile83, Gln89 or Ile91.

Particularly preferred positions for modification in VEGF monomer A: Phe17, Tyr21, Tyr25, Asp63, Leu66, Pro106, and for modification in VEGF monomer B: Lys48, Met81, Ile83 or Ile91.

Most preferred positions for modification in VEGF monomer A: Phe17, Tyr21, Asp63 or Pro106, and for modification in VEGF monomer B: Lys48, Met81, Ile83 or Ile91.

Alternatively, for modification of the second receptor-binding site, residues within 5 Å of the receptor molecule are major targets for mutagenesis:

In VEGF monomer A: Ile46, Lys48, Gln79, Met81, Ile83, Pro85, His86, Gln89 or Ile91, and in VEGF monomer B: Phe17, Met18, Tyr21, Gln22, Tyr25, Cys26, Asp63, Gly65, Leu66, Glu103, Cys104, Arg105 or Pro106.

More preferred positions for modification in VEGF monomer A: Ile46, Lys48, Gln79, Met81, Ile83, Pro85, His86, Gln89 or Ile91, and in VEGF monomer B: Phe17, Met18, Tyr21, Gln22, Tyr25, Asp63, Gly65, Leu66, Glu103, Arg105 or Pro106.

Still more preferred positions for modification in VEGF monomer A: Ile46, Lys48, Gln79, Met81, Ile83, Pro85, His86, Gln89 or Ile91, and in VEGF monomer B: Phe17, Met18, Tyr21, Gln22, Tyr25, Asp63, Leu66, Glu103, Arg105 or Pro106.

Particularly preferred positions for modification in VEGF monomer A: Lys48, Met81, Ile83 or Ile91, and in VEGF monomer B: Phe17, Tyr21, Tyr25, Asp63, Leu66 or Pro 106.

Most preferred positions for modification in VEGF monomer A: Lys48, Met81, Ile83 or Ile91, and in VEGF monomer B: Phe17, Tyr21, Asp63 or Pro 106.

In order to provide satisfactory inhibition of VEGF receptor activation, one or more (such as two or three) of the following amino acid residues in each monomer may be substituted:

F17 of the first VEGF monomer substituted by R,K,D,E,N,Q,H,Y,G,A,S,T or P;

Y21 of the first VEGF monomer substituted by R,K,D,E,N,Q,H,F,G,A,S,T or P;

D63 of the first VEGF monomer substituted by N,Q,K,R,Y,H,G,A,S,T or P;

P106 of the first VEGF monomer substituted by R,K,Q,N,D,E,G,A,S,T,P;

K48 of the second VEGF monomer substituted by R,Q,N,D,E,Y,G,A,S,T, P;

M81 of the second VEGF monomer substituted by R,K,D,E,Q,N,Y,G,A,S,T or P;

I83 of the second VEGF monomer substituted by R,K,D,E,Q,N,Y,G,A,S,T or P; and/or

I91 of the second VEGF monomer substituted by R,K,D,E,Q,N,Y,G,A,S,T or P.

Alternatively,

F17 of the second VEGF monomer is substituted by R,K,D,E,N,Q,H,Y,G,A,S,T or P;

Y21 of the second VEGF monomer is substituted by R,K,D,E,N,Q,H,F,G,A,S,T or P;

D63 of the second VEGF monomer is substituted by N,Q,K,R,Y,H,G,A,S,T or P;

P106 of the second VEGF monomer is substituted by R,K,Q,N,D,E,G,A,S,T,P;

K48 of the first VEGF monomer is substituted by R,Q,N,D,E,Y,G,A,S,T, P;

M81 of the first VEGF monomer is substituted by R,K,D,E,Q,N,Y,G,A,S,T or P;

I83 of the first VEGF monomer is substituted by R,K,D,E,Q,N,Y,G,A,S,T or P; and/or

I91 of the first VEGF monomer is substituted by R,K,D,E,Q,N,Y,G,A,S,T or P.

More preferred substitutions are the following:

F17: R,K,D,E

Y21: R,K,D,E

D63: N,R,G,A,S,T

P106: R,K,D,E

in either monomer combined with

K48: R,E,D,Q,N,Y

M81: R,K,D,E,Q,N

I83: R,K,D,E,Q,N

I91: R,K,D,E,Q,N

in the other monomer.

Most preferred substitutions are the following:

F17: R,K,D,E

D63: N

in either monomer combined with

K48: R,D,E

M81: R,K,D,E

I83: R,K,D,E

I91: R,K,D

in the other monomer.

Optionally, the N-glycosylation site at Asn75 can be substituted with Gln, Thr, Asp, or Ala in one or both monomers.

Optionally, one of the intra-chain disulfide-bonds can be removed by substituting Cys51 in one monomer with Ser concomitantly with substituting Cys60 in the other monomer with Ser.

Introduction of Glycosylation Sites in the Receptor-Binding Region:

Single-chain VEGF-heterodimers with one functional and one non-functional receptor-binding site acting as VEGF-antagonists can also be obtained by selectively introducing an N-glycosylation site in the receptor-binding site of only one of the VEGF monomers.

Preferred positions for introducing the Asn residue of an N-glycosylation site are Phe17, Tyr21, Tyr25, Lys48, Asp63, Met81, Ile83, and Ile91. In addition to substituting one or more of these amino acid residues with Asn-residues, it is also in most cases necessary to substitute the amino acid residues two positions further along the polypeptide chain with a Ser- or Thr-residue in order to create the Asn-Xaa-Ser/Thr consensus sequence for N-glycosylation. In one example, it is necessary to substitute a Pro-residue one position along the polypeptide chain with another amino acid residue as Asn-Pro-Ser/Thr sequences do not function as glycosylation sites.

Thus, the following preferred substitutions may be carried out in either one of the VEGF monomers in order to provide satisfactory inhibition of VEGF receptor activation through introduction of novel N-glycosylation sites:

Phe17Met18Asp19→Asn17Met18Thr19 or Asn17Met18Ser19

Tyr21Gln22Arg23→Asn21Gn22Thr23 or Asn21Gln22Ser23

Tyr25Cys26His27→Asn25Cys26Thr27 or Asn25Cys26Ser27

Lys48Pro49Ser50→Asn48Ala49Thr50 or Asn48Ala49Ser50

Asp63Glu64Gly65→Asn63Glu64Thr65 or Asn63Glu64Ser65

Met81Arg82Ile83→Asn81Arg82Thr83 or Asn81Arg82Ser83

Ile83Lys84Pro85→Asn83Lys84Thr85 or Asn83Lys84Ser85

Ile91Gly92Glu93→Asn91Gly92Thr93 or Asn91Gly92Ser93

More preferred substitutions are:

Phe17Met18Asp19→Asn17Met18Thr19 or Asn17Met18Ser19

Tyr21Gln22Arg23→Asn21Gln22Thr23 or Asn21Gln22Ser23

Lys48Pro49Ser50→Asn48Ala49Thr50 or Asn48Ala49Ser50

Met81Arg82Ile83→Asn81Arg82Thr83 or Asn81Arg82Ser83

Ile83Lys84Pro85→Asn83Lys84Thr85 or Asn83Lys84Ser85

Ile91Gly92Glu93→Asn91Gly92Thr93 or Asn91Gly92Ser93

Most preferred substitutions are:

Phe17Met18Asp19→Asn17Met18Thr19

Tyr21Gln22Arg23→Asn21Gln22Thr23

Lys48Pro49Ser50→Asn48Ala49Thr50

Met81Arg82Ile83→Asn81Arg82Thr83

Ile83Lys84Pro85→Asn83Lys84Thr85

Ile91Gly92Glu93→Asn91Gly92Thr93

Introduction of Selective PEGylation Sites in the Receptor-Binding Region:

Single-chain VEGF-heterodimers with one functional and one non-functional receptor-binding site acting as VEGF-antagonists can also be obtained by selectively introducing a Cys-residue in the receptor-binding site of only one of the VEGF monomers for PEGylation using thiol-selective PEGs.

Preferred positions for introducing Cys-residues in either one of the VEGF monomers for selective PEGylation are Phe17, Tyr21, Tyr25, Lys48, Asp63, Leu66, Met81, Ile83, and Ile91.

Most preferred positions are Phe17, Tyr21, Lys48, Leu66, Met81, Ile83, and Ile91.

The invention will be further illustrated by the following non-limiting examples.

Materials and Methods

Accessible Surface Area (ASA)

The computer program Access (B. Lee and F. M. Richards, J. Mol. Biol. 55: 379-400 (1971)) version 2 ((1983 Yale University) is used to calculate the accessible surface area (ASA) of the individual atoms in the relevant structure. Typically, this method uses a probe-size of 1.4 Å and defines the Accessible Surface Area (ASA) as the area formed by the center of the probe. Prior to this calculation all water molecules, all hydrogen atoms, and other atoms not directly related to the protein are removed from the coordinate set.

Determination of Association Domain

Calculating the ASA of the free monomer molecule and comparing with calculations including each of the two other monomer molecules in the trimer allows determination of residues containing atoms having their ASA changed upon trimerization. By including the other monomers one at a time it is possible to determine residues involved in interaction with each of the other monomer or with both other monomers. These residues are defined as constituting the association domain.

Determination of Receptor-Binding Site

Calculating the ASA of the free monomer molecule and comparing with calculations including each of the two receptor molecules interacting with the monomer allows determination of residues containing atoms having their ASA changed upon trimerization. By including the receptor molecules one at a time it is possible to determine residues involved in interaction with each of the two receptor molecules. The receptor-binding site of a cytokine ligand of the TNF-family, such as TNF-α, LT-α or LT-β, is defined as comprising all residues having an atom at a distance of 5.0 Å or less from an atom in a residue in one of the receptor molecules. Additional residues having their accessible surface area changed upon receptor binding (as determined from the model) also comprise the receptor-binding site. Finally the results for each monomer are combined to eliminate small differences due to the eventual non-symmetry of the system.

Determination of Association Domain and Receptor-Binding Site for LT-β

This determination is based on the sequence alignment of LT-β with TNF-α and LT-α in Table 3. Residues of an association domain are defined as those residues occupying an equivalent position in either the association domain of TNF-α or of LT-α or both. Likewise, the residues at the receptor-binding site are defined as those residues where the residue occupying an equivalent position in the receptor-binding site of either TNF-α or LT-α or both. The amino acid sequence of human LT-β is given in SEQ ID NO:3.

Structure Based Sequence Alignment of TNF-α and LT-α

A sequence alignment based on a structural alignment of the structures of molecule 1 of the TNF-α structure (“TNFA”) and molecule 1 of the LT-α structure (“LT-A”) is shown in Table 2 below. Residues at equivalent positions are approximately at the same structural positions. The first residue in the LT-α structure is Lys28, the first residue in the TNF-α structure is Arg6.

TABLE 2
Structure-based sequence alignment of TNF-αand
LT-α

LT-A
KPAAHLIGDPSKQNSLLWRANTDPAFLQDGFSLSNNSLLVPTSGT
TNFA
RTPSDKPVAHVVANPQAEGQLQWLNRRANALLANGVELRDNQLVVPSEGL
LT-A
YFVYSQVVFSGKAYSPKATSSPLYLAHEVQLFSSQYPFHVPLLSSQKMVY
TNFA
YLIYSQVLFKGQG----CPSTHVLLTHTTSRIAVSYQTKVNLLSAIKSPC
LT-A
P-G------LQEPWLHSNYHGAAFQLTQGDQLSTHTDGTPHLVL-SPSTV
TNFA
-QRETPEGAEAKPWYEPTYLGGVFQLEKGDRLSAEINRPDYLLFAESGQV
LT-A
FEGAFAL
TNFA
YFGIIAL

Alignment of the Sequence of LT-β to the Structure Based Sequence Alignment of TNF-α and LT-α

The sequence of LT-β (“LT-B”) was aligned to the alignment in Table 2 using ClustalW ver 1.74. The option Profile/Structure Alignments are used to align the sequence of LT-β to the fixed alignment of LT-α to TNF-α, resulting in the alignment in Table 3. As in Table 2, the first residue in the LT-α structure is Lys28 and the first residue in the TNF-α structure is Arg6. The first residue in the LT-β sequence is Lys 73.

TABLE 3
Alignment of the sequence of LT-βto the structural based
sequence alignment of TNF-αand LT-α.

| | |
|---|---|
| LT-A | KP---------AAHLTGDPSKQNSLLWRANTDRAFLQDGFSLSNN |
| LT-B | KLPEEEPETDLSPGLPAAHLIGAPLKGQGLGWETTKEQAFLTSGTQFSDA |
| TNFA | RTPSDKP---------VAHVVANPQAEGQLQWLNRRANALLANGVELRDN |
| LT-A | S-LLVPTSGIYFVYSQVVFSGKAY--SPKATSSPLYLAHEVQLFSSQYPFH |
| LT-B | EGLALPQDGLYYLYCLVGYRGRAPPGGGDPQGRSVTLRSSLYRAGGAYGPG |
| TNFA | Q-LVVPSEGLYLTYSQVLFKGQG------CPSTHVLLTHTISRIAVSYQTK |
| LT-A | VP--LLSSQKNVYP-G------LQEPWLHSMYHGAAFQLTQGDQLSTHTD |
| LT-B | TPELLLEGAETVTPVLDPARRQGYGPLWYTSVGFGGLVQLRRGERVYVNIS |
| TNFA | VN--LLSAIKSPC-QRETPE-GAEAKPWYEPIYLGGVFQLEKGDRLSAETN |
| LT-A | GIPHLVL-SPSTVFFGAFAL |
| LT-B | HPDMVDF-ARGKTFFGAVMVG |
| TNFA | RPDYLLFAESGQVYFGIIAL |

Identification of Association Domain

A) TNF-α

The trimer association domain in the TNF-α structure (TNFA) starting from molecule A was calculated to be: L57, K98, Q102, R103, E116, Y119 and L157 having interactions to both other monomers, R6, T7, P8, S9, Ku1, V13, A14, H15, A33, N34, L36, Y59, Q61, L63, G68, C69, C101, E104, P106, G108, A109, K112, P113, W114, Y115, P117, L143, E146, S147, G148, Q149, Y151, I154, I155 and A156 only having interactions to molecule C and E53, G54, L55, T72, H73, L75, R82, Y87, V91, N92, L93, L94, S95, A96, I97, S99, I118, L120, G121, G122, V123, F124 and Q125 only having interactions to molecule B.

B) LT-α

The trimer association domain in the LT-α structure (LT-A) starting from molecule A was calculated to be: F74, K119, H131, Y134, F169 and L171 having interactions to both other monomers, A30, A31, H32, D50, R51, F53, L54, Q55, Y76, Q78, V80, V121, E127, P128, W129, L130, S132, V158, P161, S162, T163, F165, A168 and A170 having interactions only to one of the other monomers (equivalent to molecule C in TNFA) and I72, P94, Y96, L103, S105, Q107, Y108, V112, P113, L114, L115, S116, S117, Q118, M120, Y122, M133, H135, G136, A137, A138, F139, Q140, L141, T142 and D145 having interactions only to the other monomer (equivalent to molecule B in TNFA).

C) LT-β

The trimer association domain in LT-β was determined as: K73, L74, P75, E76, E78, A89, A90, H91, E109, Q110, F112, L113, T114, D130, G131, L132, Y134, Y136, L138, G140, A145, P152, R155, S156, T158, R165, G167, A169, Y170, T174, P175, L177, L178, L179, E180, G181, A182, E183, T184, V185, T186, V188, L189, D190, A192, Q195, G196, P199, L200, W201, Y202, T203, S204, V205, G206, F207, G208, G209, L210, V211, Q212, L213, R214, E217, D230, A232, R233, G234, K235, F237, A240, V241, M242 and V243.

Identification of Receptor Interface

The receptor-binding site in LT-β was determined as K73, I93, P96, L97, G99, E105, T106, T107, K108, E109, Q110, A111, F112, D130, G140, R142, G143, R144, P146, G154, R155, S156, V157, T158, R160, R165, G167, G168, A169, Y170, G171, P172, T174, A182, T184, Y197, G198, P199, L200, W201, Y202, Q212, R214, N222, S224, H225, P226, D227, V229, D230, F231, A232, R233, and K235.

TNF Assay Outline

It has previously been published that TNF-α interacts with and activates TNF-α receptors on HeLa cells. Consequently, transcription is activated at promoters containing a NF-κ-B transcription-binding element. It is thus possible to screen for antagonists of TNF receptors by use of a NF-κ-B coupled luciferase reporter gene placed in HeLa cells.

Primary Screen:

HeLa cells are co-transfected with NF-κB ISRE-Luc (Stratagene, La Jolla, Calif., USA) and pcDNA 3.1/hygro (Invitrogen, Carlsbad, Calif., USA) and cell colonies are created by selection in media containing Hygromycin B. Cell clones are screened for luciferase activity in the presence or absence of TNF. A clone showing the highest ratio of stimulated to unstimulated luciferase activity is used in further assays.

To screen muteins for antagonist activity, 10,000 cells/well are seeded in 96 well white cell culture plates (Packard) in media w/o phenol red and incubated overnight. Muteins are added to the wells in various concentrations. Subsequently, a constant amount of TNF-α giving rise to 70-90% of maximum luciferase activity is added to all wells and the plates are incubated for 5 hours. Measurement of agonist activity is performed in the absence of this fixed concentration of TNF-α. Plates are sealed after addition of LucLite substrate (Packard Bioscience, Groningen, The Netherlands) and luminescence is measured on a TopCount (Packard) in SPC (single photon counting) mode. Each individual plate will contain wells incubated with different TNF-α concentrations as stimulated controls and other wells containing normal media as an unstimulated control. The ratio between stimulated and unstimulated luciferase activity serves as an internal standard for both mutein activity and experiment-to-experiment variation.

Secondary Assays

In addition to stimulating NF-κB transcription, TNF-α causes apoptosis through a second, non NF-κ-B dependent, pathway. To ensure that TNF antagonists are able to block all TNF activity, the ability of TNF antagonists to block TNF-α induced apoptosis of HeLa cells is demonstrated. 5,000 HeLa cells per well are seeded in a 96 well clear cell culture plate (NUNC) and incubated with antagonists and TNF-α in a concentration giving rise to 70-90% cell death for 24 hours in media containing 25 μg/ml cyclohexamide. Measurement of agonist activity is performed in the absence of this fixed concentration of TNF-α. The tetrazolium salt WST-1 (Boehringer Mannhiem, Germany), is used to assay cell viability. WST-1 is cleaved by mitochondrial dehydrogenases to a formazan dye that absorbs 450 nm light. The optical density at 450 nm is thus directly proportional to the amount of viable cells. Those muteins displaying TNF-α antagonism, defined as the ability to block TNF-α dependent NF-κB and apoptosis activities, are selected for further study.

Assays to Differentiate Between Antagonism of TNF-Receptors p55 and p75

Biochemical:

The binding of single chain TNF-α and its muteins to TNF-receptors is directly assayed using cross-linking analysis. Soluble TNF-receptors truncated at the transmembrane domain are expressed and purified. These receptors are incubated with single chain TNF-α and cross-linked using EGS, an agent previously shown to cross link TNF-α to its receptors. The cross-linked complex is analyzed by western blot using TNF and receptor specific antibodies. Based on the size of the complex molecule, the precise number (if any) of receptors bound is determined. Furthermore, the relative specificity of single chain TNF-α for p55 and p75 is determined.

A similar analysis is performed under native conditions by using gel chromatography to separate receptor-TNF complexes. Again, the size and amount of eluted proteins provides insight into the number of receptor molecule bound and the relative affinity of single chains for p55 and p75.

Another strategy is based on the BIACORE® instrument. (Zhou et al., Biochemistry, 1993, 32, 8193-98; Faegerstram and O'Shannessy, 1993, In Handbook of Affinity Chromatography, 229-52, Marcel Dekker, Inc., NY). The BIACORE® technology allows one to bind receptor, ligand or antibody directed towards any of these to a gold surface and to flow ligand or soluble receptor over it. Plasmon resonance detection gives direct quantification of the amount of mass bound to the surface in real time. This technique yields both on and off-rate constants and thus a TNF-receptor or dissociation constant and affinity constant can be directly determined, and it also enables quantification of the number of receptors bound per ligand and vice-versa.

Binding of the muteins to TNF-R is studied using standard binding assays. The receptors may be purified extracellular receptor domains, or receptors bound to purified cellular plasma membranes. The ability of the muteins to compete for the binding sites with native TNF is analyzed by incubating with a labeled TNF-analog, for instance biotinylated TNF-α or radioiodinated TNF-α.

The extracellular domains of TNF-R, optionally coupled to Fc, are immobilized in 96 well plates. Single chain TNF muteins are subsequently added and the binding of these detected using either specific anti-TNF antibodies or biotinylated or radioiodinated TNF-R. Comparison of the amount of bound single chain TNF detected with antibody and with labeled receptor enables estimation of the number of active binding sites and the level of interaction with the two receptor subtypes.

Cellular:

Several cell lines have been identified that predominantly express p55 or p75. For example, 80% of TNF receptors on Hep-2 cells are p55 and 80% of TNF receptors on HL60 cells are p75. Receptor-binding assays are performed using radiolabelled single chain TNF-α. Similarly, signal transduction experiments are targeted to demonstrate whether one or both receptor subtypes are blocked. Alternatively, cells which do not express TNF-R or express these in very low numbers can be transfected with cDNAs encoding either or both receptors and subsequently used in these studies.

TNF-α Sandwich ELISA

This assay is based on the combination of polyclonal rabbit anti-TNF-α and polyclonal goat anti-TNF-α antibodies and a specific secondary antibody conjugate.

Microtiter plates are incubated overnight at room temperature (RT) with 100 μl/well Rabbit Anti-TNF-α antibody (10 μg/ml) in sodium carbonate buffer (pH 9.6). After wash, the plates are incubated for 1 hr at RT in 100 μl/well Tris buffer (pH 7.2) containing 0.05% Tween-20 and 1% non-fat milk (THTM) to block free binding sites The plates are incubated with TNF-α containing samples in 100 μl/well THTM for 1 hr at RT. Bound TNF-α is detected by Goat Anti-TNF-α antibody (100 μl/well) (R&D Cat #AF-210-NA, dil. 1:100 in THTM) incubated for 1 hr at RT followed by peroxidase conjugated Rabbit Anti-Goat Ig (100 μl/well) (DAKO Cat #P 0160, dil. 1:4000 in THTM). The reaction is visualized by addition of 3,3′,5,5′-tetramethyl benzidine (TMB) (KEM-EN-TEC Cat #4390) (100 μl/well) for 13 minutes in the dark at RT. The reaction is stopped by addition of 100 μl/well of 1M sulfuric acid. The absorbance is read on an ELISA reader at 450 nm.

Quantification of Single-Chain Molecules

It is possible to use the BIACORE® system (Biacore, Uppsalla, Sweden) to quantify the amount of protein present in a TNF single-chain sample. Samples are flowed over a chip containing TNF antibodies and the single-chain concentration is determined based on known standards.

Quantification of Receptor Binding

Two well-based receptor-binding assays are used to determine the strength and number of receptor binding sites on single-chain muteins. In the first assay, hydrophobic wells are coated with 0.1 μg/ml soluble TNF-R1 or TNF-R2 and incubated with a dilution series of single-chain and monomeric molecules. The bound TNF is detected using a polyclonal TNF antibody from goat (R and D Systems, Minneapolis, Minn., USA) and a horse radish peroxidase-conjugated secondary antibody (Dako, Copenhagen, Denmark). As the receptors in the wells are sparse, it is presumed that each single-chain molecule binds only one receptor. Under this assumption, the resulting data describe the binding strength of the strongest site of the single-chain molecule.

A similar assay can be used to determine the relative numbers of binding sites on single-chain molecules. Single-chain muteins are bound to receptor as above but detected with biotinylated Enbrel®, a dimeric TNF-R2 construct. The amount of Enbrel® bound represents the number of binding sites remaining after TNF-R1 capture. We have observed a large difference in Enbrel® binding in wild type and certain mutein single chains; these data indicate that the mutations have destroyed the second and third binding sites in these cases.

Functional in vivo Half-Life/Clearance

The functional in vivo half-life of single chain TNF muteins is studied using a mouse model. A therapeutic dose of a TNF antagonist is injected subcutaneously, i.v. or i.p. Subsequently, TNF-α dosages equivalent to LD-50 are injected after different time intervals. The time at which 75% of the mice are still tolerant to the TNF-α injection provides an estimate of the functional in vivo half-life of the antagonist.

### EXAMPLES

### Example 1

1.1 Construction of scTNFα

Single-chain tumor necrosis factor α, scTNFα, consists of three copies of TNFα, optionally linked by two identical oligopeptide linkers. A fully synthetic DNA construct for the expression of scTNFα in Saccharomyces cerevisiae was generated in a number of polymerase chain reactions using overlapping oligonucleotides as templates (in principle as described by Stemmer, W P et al. (1995) Gene 164, 49-53). The codon usage in the synthetic scTNFα was optimized for expression of the protein in S. cerevisiae. To produce scTNFα for secretion in S. cerevisiae, the prepro sequence of S. cerevisiae α-factor (see e.g. U.S. Pat. No. 4,870,008, U.S. Pat. No. 4,546,082) was added upstream of scTNFα. The DNA and protein sequences of the resulting construct (minus the prepro sequence of S. cerevisiae α-factor) are given in SEQ ID NO:1 and SEQ ID NO:2, respectively. In addition, the DNA sequence of the construct included a BamHI site (GGATCCACG) immediately upstream of the prepro sequence as well as a XbaI site (TCTAGA) immediately downstream of the third copy of TNFα. The DNA fragment was subcloned BamHI/XbaI into yeast expression vector pJSO37 (Okkels, J S (1996) Ann. New York Acad. Sci. 782, 202-207), resulting in plasmid pBvdH1005.

In SEQ ID NO: 2, the first copy of TNF-α comprises amino acid residues at positions 1-157, the first linker comprises amino acid residues at positions 158-171, the second copy of TNF-α comprises amino acid residues at positions 172-328, the second linker comprises amino acid residues at positions 329-342, and the third copy of TNF-α comprises amino acid residues at positions 343-499. The nucleic acid positions for the corresponding codons in SEQ ID NO:1 are 1-471, 472-513, 514-984, 985-1026 and 1027-1497, respectively.

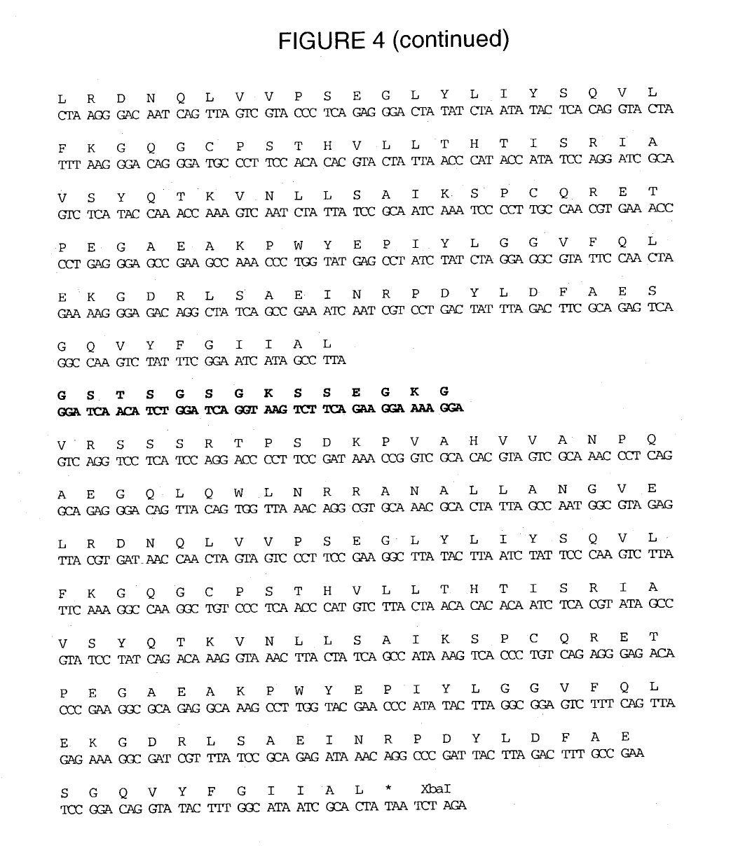

For reference purposes, the complete DNA and amino acid sequence of the scTNFα construct, including the prepro sequence of S. cerevisiae α-factor, and with the amino acid residues (one-letter code) aligned with the corresponding codon, is given in FIG. 4. In FIG. 4, the sequences of the yeast α-factor prepro sequence, the three copies of TNFα, and the two linker regions are shown in italics, regular, and bold font styles, respectively. Also shown are the nucleic acid sequences immediately before the prepro sequence (BamHI) and after the third copy of TNFα (XbaI).

As an alternative for extracellular production in S. cerevisiae, the signal peptide of the S. cerevisiae gene YAP3 (WO 98/32867) and the synthetic leader sequence TA57 (WO 98/32867) were linked to the N-terminus of the recombinant protein. Furthermore, as an alternative to yeast expression, scTNFα was directed towards the periplasmic compartment in E. coli, by insertion of scTNFα behind the ompT signal peptide (Grodberg et al., Nucleic Acids Res. 16(3), 1209 (1988)) in plasmid pET12a (Novagen Inc.). To facilitate purification, scTNFα was also expressed in either E. coli or S. cerevisiae with a covalently linked N-terminal histidine tag.

1.2 Expression of scTNFα in Saccharomyces cerevisiae

S. cerevisiae YNG318 (available from the American Type Culture Collection, VA, USA as ATCC 208973) was transformed with plasmid pBvdH1005 using a standard lithium acetate procedure (Gietz et al. 1992 Nucleic Acids Res. 6, 1425). Transformants were selected by growth on selective media lacking uracil (7.5 g per litre yeast nitrogen base w/o amino acids (Difco), 11.3 g per litre Bernstein acid (Merck), 6.8 g per litre NaOH (Merck), 5.6 g per litre casamino acid w/o vitamin, 0.1 g per litre tryptophan, 20 g per litre glucose (Sigma), 0.1 g per litre threonine, 2 g per litre bacto-agar). For large-scale expressions, a single transformant colony was inoculated in 10 ml liquid selective medium. After overnight growth at 30° C., 5 ml of this culture was diluted into 100 ml fresh selective media. After another overnight incubation at 30° C., 50 ml of the 100 ml culture was diluted into 5 liters of YPD media (1% w/w yeast extract (Difco), 2% w/w peptone (Difco), 3% w/w dextrose (Roquette)) and grown for 48 hrs at 30° C. Cells were removed from the culture medium by centrifugation (15 min at 5000 rpm in a Sorvall RC5C centrifuge) and the supernatant was microfiltrated using a 0.22 μm filter prior to purification.

1.3 Purification of scTNFα Molecules Produced in S. cerevisiae

The microfiltrated culture supernatant was concentrated app. 20 times using Vivaflow 200 (Vivascience) ultrafiltration equipment and a 30 k cut-off membrane. To the concentrated supernatant was then added “protease inhibitor cocktail tablets” (Roche)- one tablet per 50 ml concentrated cell extract-and EDTA to obtain a final concentration of 5 mM EDTA. Before application, the pH was adjusted to approximately 6.8 and the conductivity determined (≦5 mS/cm at room temperature).

At low ionic strength and pH 6.8 (or lower) scTNFα binds to a Toyopearl SP-550C cation exchange resin (TosoHaas) equilibrated in 20 mM sodium phosphate, 5 mM EDTA, pH 6.8 (buffer A). After application, the cation exchanger used at the capture step was washed with at least 3 column volumes of buffer A (until the absorbance at 280 nm reaches baseline level) and scTNFα was isocratically eluted with buffer A including 0.5 M NaCl.

The obtained eluate was first concentrated 10-20 times in Vivaspin 15, Mw 5000, ultrafiltration modules or equivalent in order to reduce the volume and then diluted app. 25 times with distilled water to obtain a low ionic strength (≦4 mS/cm at room temperature). Prior to application on the next chromatographic column Tween 20 was added to 0.05% (v/v) and the pH adjusted to 5.5.

In the second purification step the pH adjusted eluate pool from the Toyopearl SP-550C column was applied on a Mono S cation exchanger column (Pharmacia) equilibrated in 20 mM sodium acetate, 0.05% (v/v) Tween 20, pH 5.5. The cation exchanger column was then washed with at least 3 column volumes of buffer A (until the absorbance at 280 nm reaches baseline level) and scTNFα eluted with a gradient from buffer A to buffer A including 1 M NaCl (buffer B).

If necessary, further purification of scTNFα may be investigated using immobilized TNF receptor1/receptor2, Size Exclusion Chromatography (SEC) and/or Reverse Phase HPLC (RP-HPLC).

Purity, identity, quantity and activity of eluted fractions from the above mentioned columns can be determined using a combination of methods known by the person skilled in the art. These may include one or more of the following assays and methods or other relevant methods known by the person skilled in the art: the primary and secondary assays described above, ELISA methods, BIACORE®, SDS-PAGE, western blotting, IEF, HPLC, SEC, amino acid sequencing, mass spectrometry, peptide mapping and amino acid analysis.

1.4 Biological Activity of scTNFα

Purified scTNFα was assayed in the primary screening assay as described under Materials and Methods. When assayed for agonist activity, scTNFα gives rise to the same maximum stimulation of luciferase activity as TNFα (FIG. 1). Based on this finding, it was concluded that scTNFα behaves as a full agonist.

### Example 2

Construction of Variants of scTNFα and their Expression in S. cerevisiae

Variants of scTNFα containing mutations at various positions in the molecule were constructed by PCR using pBvdH1005 (scTNFα wildtype) as a template and mutagenic oligonucleotides containing the desired mutations as primers (following the principle described by Vallejo et al. (1995) In: PCR primer, A Laboratory Manual, Cold Spring Harbor Laboratory Press, Ed.: Dieffenbach and Dveksler, pp 603-612). The PCR products, consisting of a fragment of scTNFα containing the desired mutation, were digested using suitable restriction enzymes and used to replace the corresponding DNA fragment in pBvdH1005. Transformation of S. cerevisiae YNG318 and large-scale expressions and purifications of variants were performed as described in Example 1.

In order to generate a TNFα antagonist, residues Tyr87 in copy 1 and Tyr87 in copy 3 of scTNFα were mutated to Arg, resulting in a scTNFα variant termed Y87R(1)(3). The plasmid expressing variant Y87R(1)(3) was termed pBvdH1170.

Optionally, in order to reduce undesired partial proteolytic processing around positions Arg31 and Arg32 of scTNFα, these residues may be mutated to non-basic residues.

Biological Activity of scTNFα Variants

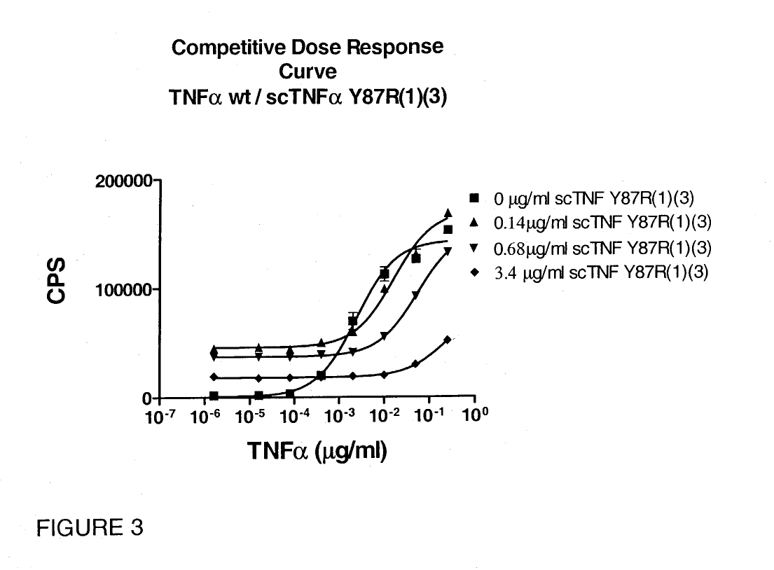

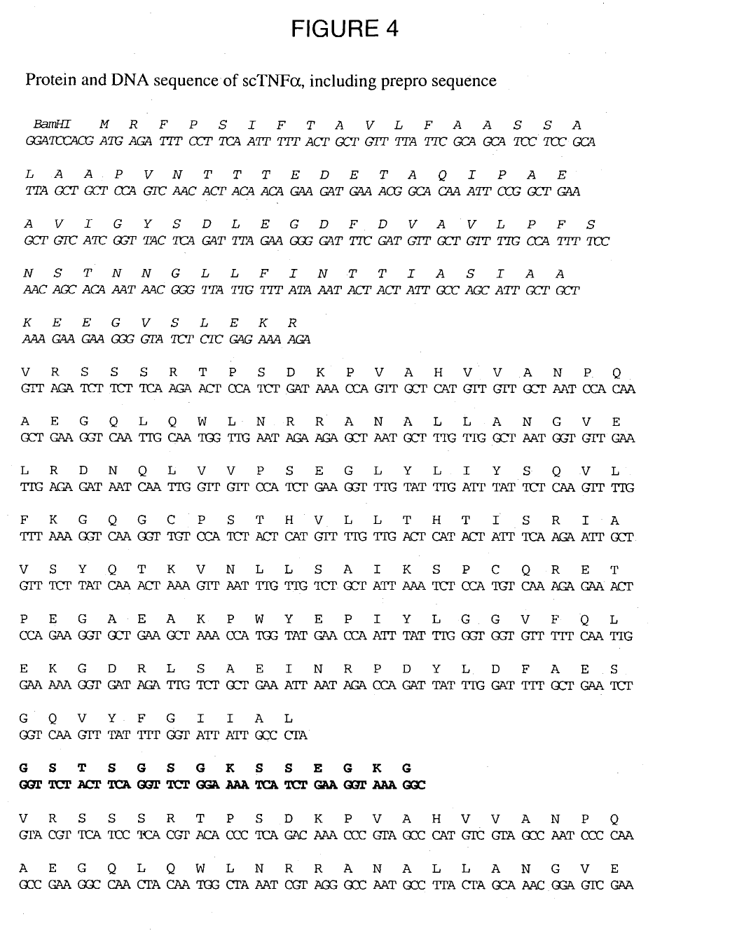

The purified variant scTNFα Y87R(1)(3) was assayed in the primary screening assay (as described under Materials and Methods). As shown in FIG. 1, this variant displays a bell-shaped dose-response curve with about a 5 fold reduction in maximum stimulation of luciferase activity compared to TNFα monomer wildtype (wt) and scTNFα wt. scTNFα Y87R(1)(3) is able to inhibit the TNFα signal in a concentration dependent manner when assayed in the presence of 0.01 μg/ml TNFα monomer (FIG. 2). Full TNFα dose-response curves were determined in the presence of various fixed concentrations of scTNFα Y87R(1)(3) in order to demonstrate the competitive nature of this inhibition. The results from this experiment (presented in FIG. 3) clearly shows a rightward shift of the TNFα concentration response with increasing concentrations of scTNFα Y87R(1)(3). (The increase seen in the basal activity (i.e. at low TNFα concentrations) with decreasing concentrations of scTNFα Y87R(1)(3) is a reflection of the weak bell shaped agonistic activity intrinsic to this variant). On the basis of these results it is concluded that scTNFα Y87R(1)(3) is a partial TNFα agonist and a competitive TNF-receptor antagonist.

Several other scTNFα variants have also been shown to behave as partial antagonists with corresponding partial agonistic activity.

All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A single-chain oligomeric polypeptide which binds to an extracellular ligand-binding domain of a cellular receptor of a type requiring binding of an oligomeric ligand to two or more receptor subunits to be activated, the polypeptide comprising at least three receptor-binding sites of which at least one is capable of binding to a ligand-binding domain of the cellular receptor and at least one is incapable of effectively binding to a ligand-binding domain of the cellular receptor, whereby the single-chain oligomeric polypeptide is capable of binding to the receptor, but incapable of activating the receptor.

2. A single-chain oligomeric polypeptide which binds to an extracellular ligand-binding domain of a cellular receptor of a type requiring binding of an oligomeric ligand to two or more receptor subunits to be activated, the polypeptide comprising at least two structurally homologous receptor-binding sites of which at least one is capable of binding to a ligand-binding domain of the cellular receptor and at least one is incapable of effectively binding to a ligand-binding domain of the cellular receptor, whereby the single-chain oligomeric polypeptide is capable of binding to the receptor, but incapable of activating the receptor.

3. The polypeptide of claim 1, wherein the receptor-binding sites are located in different, symmetrical regions of the polypeptide.

4. The polypeptide of claim 1, wherein each receptor-binding site includes one or more amino acid residues originating from a first monomer and one or more amino acid residues originating from a second monomer of the oligomeric polypeptide.

5. The polypeptide of claim 1, wherein the receptor-binding sites are located at interfaces between monomeric constituents of the oligomeric polypeptide.

6. The polypeptide of claim 1, wherein at least two of the receptor-binding sites are structurally homologous.

7. The polypeptide of claim 2, which is a dimer.

8. The polypeptide of claim 1, which is a trimer.

9. The polypeptide of claim 8, wherein at least two of the receptor-binding sites are structurally homologous.

10. The polypeptide of claim 8, which comprises two receptor-binding sites capable of binding to a ligand-binding domain of the cellular receptor and one modified receptor-binding site which is incapable of effectively binding to the ligand-binding domain of the cellular receptor.

11. The polypeptide of claim 8, which comprises one receptor-binding site capable of binding to a ligand-binding domain of the cellular receptor and two modified receptor-binding sites which are incapable of effectively binding to a ligand-binding domain of the cellular receptor.

12. The polypeptide of claim 10, wherein the modified receptor-binding site is incapable of effectively binding to the ligand-binding domain of the cellular receptor due to steric hindrance.

13. The polypeptide of claim 12, wherein the modified receptor-binding site is blocked by a non-polypeptide moiety.

14. The polypeptide of claim 10, wherein the modified receptor-binding site differs from its parent receptor-binding site by deletion, substitution and/or insertion of one or more amino acid residues at one or more positions of said parent receptor-binding site.

15. The polypeptide of claim 1, which is modified by introduction of an amino acid residue comprising an attachment group for a non-polypeptide moiety in such a manner that a non-polypeptide moiety conjugated to said attachment group blocks a receptor-binding site.

16. The polypeptide of claim 15, wherein the amino acid residue comprising an attachment group for a non-polypeptide moiety is introduced in a receptor-binding site.

17. The polypeptide of claim 16, wherein the amino acid residue comprising an attachment group for a non-polypeptide moiety is a cysteine residue or an asparagine residue.

18. The polypeptide of claim 15, wherein the non-polypeptide moiety is an oligosaccharide moiety or a polymer.

19. The polypeptide of claim 1, comprising first and second monomers that are each modified in at least one position forming part of the same receptor-binding site.

20. The polypeptide of claim 1, which binds to a cellular receptor selected from the group consisting of cytokine receptors, growth factor receptors, protein-tyrosine kinase receptors, death domain receptors, including the TNF receptor family, and serine-threonine kinase receptors.

21. The polypeptide of claim 20, wherein the cytokine is selected from the group consisting of IFN-γ, lymphotoxin-α, lymphotoxin-β, IL-10, IL-16, FAS ligand, TRAIL, CD40 ligand, CD30 ligand, CD27 ligand, OX40 ligand, APRIL, 4-1BB ligand, TRANCE and OPGL.

22. The polypeptide of claim 21, which is an OPGL antagonist.

23. The polypeptide of claim 20, wherein the growth factor is selected from the group consisting of VEGF, VEGF-B, VEGF-C, VEGF-D, PDGF, PlGF, TGF-β1, TGF-β2, TGF-β3, TGF-β4, bone morphogenetic protein 2 (BMP-2), BMP-3, BMP-4, BMP-5, BMP-6, BMP-7, BMP-8, growth differentiating factor 1 (GDF-1), GDF-5, GDF-8 (myostatin), GDF-10, Muellerian inhibiting factor, inhibin A, inhibin B, activin A and activin AB.

24. The polypeptide of claim 1, comprising at least one receptor-binding site with at least one modification that results in increased receptor-binding activity of said modified receptor-binding site compared to a corresponding polypeptide without said modification.

25. The polypeptide of claim 24 which is a trimer with one or two receptor-binding sites having increased receptor-binding activity.

26. The polypeptide of claim 1, wherein the monomers are linked by a linker peptide.

27. The polypeptide of claim 26, wherein the linker peptide is a sequence of at least one and not more than about 30 amino acid residues.

28. The polypeptide of claim 26, wherein the linker peptide comprises one or more amino acid residues selected from the group consisting of Gly, Ser, Ala and Thr.

29. The polypeptide of claim 1 which is a single-chain trimeric TNF receptor antagonist.

30. The polypeptide of claim 29, wherein the TNF receptor is the TNF-R1, the TNF-R2 or both of the TNF-R1 and TNF-R2.

31. The polypeptide of claim 29, wherein each of the three monomers comprised in the single-chain trimeric polypeptide is derived from a cytokine ligand of the TNF family.

32. The polypeptide of claim 29, which comprises at least one TNF-α monomer.

33. The polypeptide of claim 32, which comprises one TNF-α monomer and two monomers derived from the same or different cytokine ligand(s) of the TNF family other than TNF-α, or two TNF-α monomers and one monomer derived from a cytokine ligand of the TNF family other than TNF-α.

34. The polypeptide of claim 29, comprising three TNF-α monomers.

35. The polypeptide of claim 34, wherein receptor-binding sites 1 and 3 are inactive and receptor-binding site 2 is active.

36. The polypeptide of claim 29, which comprises at least one monomer derived from a cytokine ligand of the TNF family other than TNF-α.

37. The polypeptide of claim 36, which comprises three monomers derived from the same or different cytokine ligands of the TNF family other than TNF-α.

38. The polypeptide of claim 31, wherein the cytokine ligand of the TNF family other than TNF-α is selected from the group consisting of LT-α and LT-β.

39. The polypeptide of claim 38, which comprises a) one LT-α monomer, one LT-β monomer and one TNF-α monomer; b) two LT-α monomers and either one LT-β monomer or one TNF-α monomer; or c) two LT-β monomers and either one LT-α monomer or one TNF-α monomer.

40. The polypeptide of claim 32, wherein a receptor-binding site of TNF-α is substituted by a receptor-binding site of a cytokine ligand of the TNF family other than TNF-α.

41. The polypeptide of claim 34, which comprises a first, second and third TNF-α monomer which are linked via a peptide bond or a peptide linker so as to form a trimer, which polypeptide comprises a first, a second and a third receptor-binding site located at interfaces between the monomers, wherein
(a) said first and/or second TNF-α monomer is modified in an amino acid residue constituting part of the first receptor-binding site, and/or wherein
(b) said second and/or third TNF-α monomer is modified in an amino acid residue constituting part of the second receptor-binding site, and/or wherein
(c) said third and/or first TNF-α monomer is modified in an amino acid residue constituting part of the third receptor-binding site,
such that the polypeptide comprises one or two active receptor-binding sites capable of binding to a ligand-binding domain of a TNF receptor and one or two inactive receptor-binding sites.

42. The polypeptide of claim 41, wherein:
(a) said first TNF-α monomer is modified by substitution of one or more amino acid residues in positions 53, 71, 72, 73, 74, 75, 77, 82, 84, 85, 86, 87, 88, 89, 91, 97, 125, 127, 137 or 138, and/or wherein said second TNF-α monomer is modified by substitution of one or more amino acid residues in positions 6, 17, 20, 21, 23, 29, 30, 31, 32, 33, 34, 35, 63, 65, 66, 67, 110, 111, 112, 113, 114, 115, 140, 142, 143, 144, 145, 146, 147 or 149; and/or
(b) said second TNF-α monomer is modified by substitution of one or more amino acid residues in positions 53, 71, 72, 73, 74, 75, 77, 82, 84, 85, 86, 87, 88, 89, 91, 97, 125, 127, 137 or 138, and/or wherein said third TNF-α monomer is modified by substitution of one or more amino acid residues in positions 6, 17, 20, 21, 23, 29, 30, 31, 32, 33, 34, 35, 63, 65, 66, 67, 110, 111, 112, 113, 114, 115, 140, 142, 143, 144, 145, 146, 147 or 149; and/or
(c) said third TNF-α monomer is modified by substitution of one or more amino acid residues in positions 53, 71, 72, 73, 74, 75, 77, 82, 84, 85, 86, 87, 88, 89, 91, 97, 125, 127, 137 or 138, and/or wherein said first TNF-α monomer is modified by substitution of one or more amino acid residues in positions 6, 17, 20, 21, 23, 29, 30, 31, 32, 33, 34, 35, 63, 65, 66, 67, 110, 111, 112, 113, 114, 115, 140, 142, 143, 144, 145, 146, 147 or 149;
with the proviso that the modified polypeptide comprises one or two active receptor-binding sites capable of binding to a ligand-binding domain of a TNF receptor.

43. The polypeptide of claim 42, wherein:
(a) said first monomer is modified by substitution of one or more amino acid residues in positions 75, 87, 91 or 97, and/or said second monomer is modified by substitution of one or more amino acid residues in positions 20, 32, 65, 143 or 146; and/or
(b) said second monomer is modified by substitution of one or more amino acid residues in positions 75, 87, 91 or 97 and/or said third monomer is modified by substitution of one or more amino acid residues in positions 20, 32, 65, 143 or 146; and/or
(c) said third monomer is modified by substitution of one or more amino acid residues in positions 75, 87, 91 or 97 and/or said first monomer is modified by substitution of one or more amino acid residues in position 20, 32, 65, 143 or 146;
with the proviso that the modified polypeptide comprises one or two active receptor-binding sites capable of binding to a ligand-binding domain of a TNF receptor.

44. The polypeptide of claim 29, wherein at least one N-glycosylation site has been introduced into the amino acid sequence of the polypeptide in such a manner that the resulting polypeptide, when expressed in a glycosylating host cell, comprises an oligosaccharide moiety which is positioned at said N-glycosylation site so as to render at least one receptor-binding site inactive by means of steric hindrance.

45. The polypeptide of claim 29, comprising at least one receptor-binding site with a higher binding affinity to a ligand binding domain of a TNF receptor relative to wild-type human TNF-α.

46. The polypeptide of claim 45, wherein said at least one receptor-binding site has a higher binding affinity towards the p55 receptor, the p75 receptor or both of these relative to a corresponding receptor-binding site of a corresponding unmodified single-chain trimeric polypeptide.

47. The polypeptide of claim 29, wherein 1-10 amino acid residues at the N-terminal part of at least one of the monomers are deleted relative to wild-type human TNF-α.

48. The polypeptide of claim 29, comprising a linker peptide as shown SEQ ID NO:2.

49. The polypeptide of claim 29, which has substantially no agonist effect on at least one TNF receptor.

50. The polypeptide of claim 49, which has substantially no agonist effect on either of the TNF receptors TNF-R1 and TNF-R2.

51. The polypeptide of claim 29, which has a partial agonist effect on at least one TNF receptor.

52. The polypeptide of claim 29, which has a serum or in vivo half-life comparable to that of the ligand which it is intended to block.

53. The polypeptide of claim 29, which has a serum or in vivo half-life that is not more than about 10 times that of human TNF-α.

54. A nucleotide sequence encoding a single-chain oligomeric polypeptide according to claim 1.

55. An expression vector comprising a nucleotide sequence according to claim 54.

56. A recombinant host cell comprising a nucleotide sequence according to claim 54 or an expression vector comprising said nucleotide sequence.

57. A method for producing a nucleotide sequence according to claim 54, wherein a single nucleotide sequence encoding the single-chain polypeptide is subjected to mutagenesis so as to render at least one receptor-binding site of the polypeptide encoded by said nucleotide sequence incapable of effectively binding to a ligand-binding domain of the cellular receptor.

58. The method of claim 57, wherein the mutagenesis comprises at least one DNA shuffling cycle.

59. A method for preparing a single-chain oligomeric polypeptide according to claim 1, comprising culturing a recombinant host cell comprising a single nucleotide sequence encoding said polypeptide in a suitable culture medium under conditions permitting expression of the nucleotide sequence and recovering the resulting polypeptide from the cell culture.

60. The method of claim 59, which further comprises conjugating the polypeptide to a non-polypeptide moiety.

61. A pharmaceutical composition comprising a single-chain oligomeric polypeptide according to claim 1 together with at least one pharmaceutically acceptable excipient or vehicle.

62. A method for inhibiting activation of a cellular receptor that requires binding of an oligomeric ligand to two or more receptor subunits to be activated, comprising administering to a patient in need thereof a therapeutically effective dose of a single-chain oligomeric polypeptide according to claim 1.

63. A method for preventing or treating a disease or condition involving undesirable signal transduction from or undesirable activation of a TNF-α receptor, or for counteracting undesirable effects of endogenous or exogenous TNF-α, comprising administering to a patient in need thereof a therapeutically effective dose of a TNF receptor antagonist polypeptide according to claim 29.

64. The method of claim 63, wherein the disease or condition is an inflammatory disease or condition.

65. The method of claim 64, wherein the inflammatory disease or condition is selected from the group consisting of rheumatoid arthritis, Crohn's disease, systemic lupus erythematosus, Sjögren's disease, cachexia, diabetes mellitus, septic shock, myastenia gravis, juvenile arthritis, athero sclerosis, myocardial infarction, psoriasis, psoriasis arthritis, morbus Still, Wegener's granulomatosis, uveitis, anchylosing spondylitis, acute inflammatory conditions, post-surgical stress, and brain damage.
